(19) United States
(12) Patent Application Publication
(10) Pub. No.: US 2016/0053047 A1
(43) Pub. Date: Feb. 25, 2016

(54) PROCESSING BIOMASS

(71) Applicant: XYLECO, INC., Woburn, MA (US)

(72) Inventors: Marshall MEDOFF, Brookline, MA (US); Thomas Craig MASTERMAN, Rockport, MA (US); Jaewoong MOON, Andover, MA (US); Christopher G. BERGERON, Fitchburg, MA (US)

(21) Appl. No.: 14/758,893

(22) PCT Filed: May 16, 2014
(86) PCT No.: PCT/US2014/038341

§ 371 Date: Jul. 1, 2015

Related U.S. Application Data
(60) Provisional application No. 61/941,771, filed on Feb. 19, 2014.
(60) Provisional application No. 61/824,597, filed on May 17, 2013.

(51) Int. Cl. C08G 63/685; B01J 19/24; C07C 229/24; C12P 13/20; C12P 13/14

(52) U.S. Cl. CPC C08G 63/685; C12P 13/20; C12P 13/14; C07C 229/24; B01J 19/24; B01J 2219/00164; B01J 2219/24

(57) ABSTRACT

Biomass (e.g., plant biomass, animal biomass, and municipal waste biomass) is processed to produce useful intermediates and products, such as amino-alpha, omega-dicarboxylic acid and amino-alpha, omega-dicarboxylic acid derivatives. These products include polymers and copolymers of alpha-amino, omega-dicarboxylic acids.

This application incorporates by reference the full disclosure of the following co-pending provisional applications: U.S. Ser. No. 61/824,597, filed May 17, 2013 and U.S. Ser. No. 61/941,771 filed Feb. 19, 2014.

### BACKGROUND OF THE INVENTION

Many potential lignocellulosic feedstocks are available today, including agricultural residues, energy grasses, woody biomass, municipal waste, oilseeds/cakes and seaweed, to name a few. At present, these materials are often under-utilized, being used, for example, as animal feed, biocompost materials, burned in a co-generation facility or even landfilled.

Lignocellulosic biomass includes crystalline cellulose fibrils embedded in a hemicellulose matrix, surrounded by lignin. This produces a compact matrix that is difficult to access by enzymes and other chemical, biochemical and/or biological processes. Cellulosic biomass materials (e.g., biomass material from which the lignin has been removed) is more accessible to enzymes and other conversion processes, but even so, naturally-occurring cellulosic materials often have low yields (relative to theoretical yields) when contacted with hydrolyzing enzymes. Lignocellulosic biomass is even more recalcitrant to enzyme attack. Furthermore, each type of lignocellulosic biomass has its own specific composition of cellulose, hemicellulose and lignin.

### SUMMARY

Generally, this invention relates to methods and processes for converting a material, such as a biomass feedstock, e.g., cellulosic, starchy or lignocellulosic materials, to useful products, for example, amino-alpha, omega-dicarboxylic acids and derivatives of amino-alpha, omega-dicarboxylic acids. These amino dicarboxylic acids can be converted into other products, if desired. When the amino group is in the two position, the acid can be an amino acid, for example, an alpha-amino-alpha, omega-dicarboxylic acid. The amino group amino-alpha, omega dicarboxylic acid may be substituted on any atom on the carbon chain leading to, for example, alpha, beta, gamma, delta, and epsilon amino dicarboxylic acids. In addition, the amino dicarboxylic acids may have multiple amines in the same dicarboxylic acid. The mono-amine and the poly-amino-carboxylic acid can be substituted with other groups, e.g., alkyl groups. The carbon chain of the carboxylic acid may be straight chained, branched, cyclic, or alicyclic.

The amphiphilic nature of these structures leads to interesting properties for both low molecular products and polymeric products. The polymeric products can be amide condensation products. The amide product can be hydrolytically stable.

An amino-alpha, omega-dicarboxylic acid is shown in Structure I. This structure corresponds to an alpha-amino, alpha, omega-dicarboxylic acid.

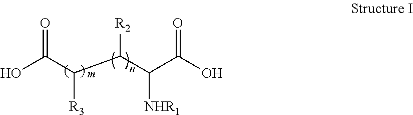

Where n and m are integers,

m=0 to 7,

n=0 to 7,

n+m≦10,

R₁=H, straight chain, branched alkyls with less than 24 carbons, aromatics, or substituted alkyl aromatics,

R₂=H, NHR₁, straight chain, branched alkyls with less than 24 carbons, aromatics or substituted alkyl aromatics,

R₃=H, NHR₁, straight chain, branched alkyls with less than 24 carbons, aromatics, or substituted alkyl aromatics.

In a preferred embodiment m=1 and n=0 and R₁ and R₃ are all hydrogen resulting in D-aspartic acid (Ia) or L-aspartic acid (Ib) shown in Structures Ib.

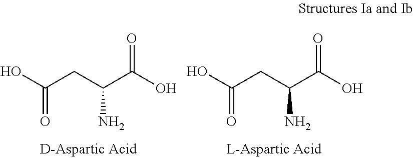

In another preferred embodiment m=1 and n=1 and R₁, R₂, and R₃ are all hydrogen resulting in D-glutamic acid (Ic) or L-glutamic acid (Id).

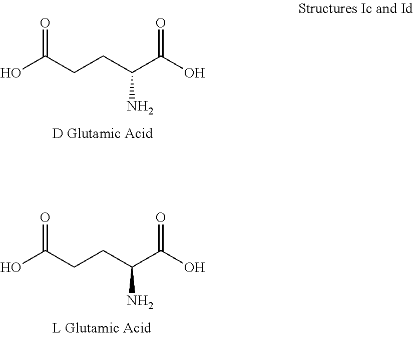 [SMILES: N[C@H](CCC(=O)O)C(=O)O ; N[C@@H](CCC(=O)O)C(=O)O]

Alternatively, the amino group can be substituted in other positions. The amino-alpha, omega dicarboxylic acid with the amino group substituted at least one group removed from the carboxylic acid is shown in Structure II

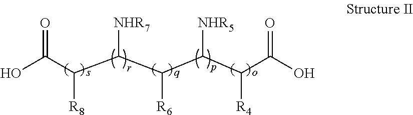

Where o, p, q, r and s are integers

o=1, 2, or 3;

p=1 or 2;

q=0, 1, 2, 3;

r=0, 1;

s=1, 2, or 3;

o+p+q+r+s≦10

R₄=H, straight chain, branched alkyls with less than 24 carbons, aromatics, or substituted alkyl aromatics,

R₅=H, straight chain, branched alkyls with less than 24 carbons, aromatics, or substituted alkyl aromatics,

R₆=H, straight chain, branched alkyls with less than 24 carbons, aromatics, or substituted alkyl aromatics,

R₇=H, straight chain, branched alkyls with less than 24 carbons, aromatics, or substituted alkyl aromatics,

R₈=H, straight chain, branched alkyls with less than 24 carbons, aromatics, or substituted alkyl aromatics.

For example, in Structure I, m is chosen from 0, 1, 2, 3, 4, 5, 6, or 7; n is chosen from 0, 1, 2, 3, 4, 5, 6, or 7; with the limitation that n+m must be less than or equal to 10; and R₁, R₂, R₃ are chosen from hydrogen, straight chain or branched alkyl groups, aromatic and alkyl aromatics where the limitation is that there are less than 24 carbons. When n+m are 10, the amino dicarboxylic acid is a derivative of dodecanoic acid where the amine group can be substituted at any of the carbon positions. Furthermore, multiple amine substitutions can occur. For the symmetric 1,10-diamino dicarboxylic acid o and p are 0; p and r are 1 and q is 8. Any combination of n, m and the R groups can be included in the alpha-amino, omega-dicarboxylic acid. Where p and r are 1 or greater there are multiple amine substituents.

In one aspect the invention relates to a method for making a product including treating a reduced recalcitrance biomass (e.g., lignocellulosic and/or cellulosic material) with one or more enzymes and/or microorganisms to produce an amino-alpha, omega-dicarboxylic acid and converting the amino-alpha, omega-dicarboxylic acid to the product. Optionally, the feedstock is pretreated with at least one method selected from irradiation (e.g., with an electron beam), sonication, oxidation, pyrolysis, size reduction, and steam explosion, for example, to reduce the recalcitrance lignocellulosic and/or cellulosic material.

Some examples of amino-alpha, omega-dicarboxylic acids that can be produced and then further converted include aspartic acid, glutamic acid and the amino substituted malonic, adipic, pimelic, suberic, azelaic and sebacic acids or their corresponding acidic or basic salts, e.g., their Na⁺, K⁺, Ca²⁺, or ammonium salts and mixtures of salts and acids.

In one implementation of the method, the amino-alpha, omega-dicarboxylic acids are converted chemically or biochemically, for example, by converting aspartic acid or glutamic acid to the respective polyamides. Other methods of chemically converting that can be utilized include polymerization, isomerization, esterification, amidation, cyclization, oxidation, reduction, disproportionation and combinations of these.

In another implementation, the lignocellulosic and/or cellulosic material is treated with one of more enzymes to release one or more sugars. For example, to release glucose, xylose, sucrose, maltose, lactose, mannose, galactose, arabinose, fructose, dimers of these such as cellobiose, heterodimers of these such as sucrose, oligomers of these, and mixtures of these. Optionally, treating can further include (e.g., subsequently to releasing sugars) utilizing (e.g., by contacting with the sugars and/or biomass) one or more organisms to produce the amino-alpha, omega-dicarboxylic acids. For example, the sugars can be fermented by a sugar fermenting organism to the amino-alpha, omega-dicarboxylic acids. Sugars that are released from the biomass can be purified (e.g., prior to fermenting) by, for example, a method selected from electrodialysis, distillation, centrifugation, filtration, chromatography, including simulated moving bed chromatography, cation exchange chromatography, and combinations of these in any convenient order.

In some implementation, converting comprises polymerizing the aspartic or glutamic acid to a polymer (e.g., polymerizing in a melt such as without an added solvent). For example, polymerizing methods can be selected from direct condensation of the aspartic or glutamic acid, azeotropic dehydrative condensation of the aspartic or glutamic acid, and cyclizing the aspartic or glutamic acid followed by ring opening polymerization. The polymerization can be in a melt (e.g., without a solvent and above the melting point of the polymer) or can be in a solution (e.g., with an added solvent). A polyamide can be a product of the polymerization process. Optionally, polymerizations can be done utilizing catalysts and/or promoters. For example, protonic acids, H₃PO₄, H₂SO₄, methane sulfonic acid, p-toluene sulfonic acid, NAFION® NR 50 H+ form from DuPont, Wilmington Del., acids supported on polymers, Mg, Al, Ti, Zn, Sn, metal oxides, TiO₂, ZnO, GeO₂, ZrO₂, SnO, SnO₂, Sb₂O₃, metal halides, ZnCl₂, SnCl₂, SnCl₄, Mn(AcO)₂, Fe₂(LA)₃, Co(AcO)₂, Ni(AcO)₂, Cu(OA)₂, Zn(LA)₂, Y(OA)₃, Al(i-PrO)₃, Ti(BuO)₄, TiO(acac)₂, (Bu)₂SnO, tin octoate, solvates and hydrates of any of these and mixtures of these can be used.

Also optionally, the polymerizations or at least a portion of the polymerizations can be done at a temperature between about 100 and about 240° C., such as between about 110 and about 200° C., optionally between about 120° C. and about 170° C., or between about 120 and about 160° C. Alternatively, at least a portion of the polymerizations can be performed under vacuum (e.g., between about 0.1 mm Hg to 300 mm Hg).

In the implementations wherein the polymerization method includes dimerizing the aspartic or glutamic acid to a lactam followed by ring opening polymerization of the lactam, the dimerization can include heating the aspartic or glutamic acid to between 100 and 200° C. under a vacuum of about 0.1 to about 100 mmHg.

Optionally, the dimerization (e.g., dimerization reaction) can include utilizing a catalyst. Catalysts can, for example, include Sn octoate, Li carbonate, Zn diacetate dehydrate, Ti tetraisopropoxide, potassium carbonate, tin powder and mixtures of these. Optionally, a ring opening polymerization catalyst is utilized. For example, the ring opening polymerization catalyst can be chosen from protonic acids, HBr, HCl, triflic acid, Lewis acids, ZnCl₂, AlCl₃, anions, potassium benzoate, potassium phenoxide, potassium t-butoxide, and zinc stearate, metals, tin, zinc, aluminum, antimony, bismuth, lanthanide and other heavy metals, tin (II) oxide and tin (II) octoate (e.g., 2-ethylhexanoate), tetraphenyl tin, tin (II) and (IV) halogenides, tin (II) acetylacetonoate, distannoxanes (e.g., hexabutyldistannoxane, R₃SnOSnR₃ where R groups are alkyl or aryl groups), Al(OiPr)₃, other functionalized aluminum alkoxides (e.g., aluminum ethoxide, aluminum methoxide), ethyl zinc, lead (II) oxide, antimony octoate, bismuth octoate, rare earth catalysts, yttrium tris(methyl lactate), yttrium tris(2-N—N-dimethylamino ethoxide), samarium tris(2-N—N-dimethylamino ethoxide), yttrium tris(trimethylsilylmethyl), lanthanum tris(2,2,6,6-tetramethylheptanedionate), lanthanum tris(acetylacetonate), yttrium octoate, yttrium tris(acetylacetonate), yttrium tris(2,2,6,6-tetramethylheptanedionate), combinations of these (e.g., ethyl zinc/aluminum isopropoxide) and mixtures of these.

After the polymerization has reached the desired molecular weight, it may be necessary to deactivate and/or remove the catalyst from the polymer. The catalyst can be reacted with a variety of compounds, including, silica, functionalized silica, alumina, clays, functionalized clays, amines, carboxylic acids, phosphites, acetic anhydride, functionalized polymers, EDTA and similar chelating agents.

While not being bound by theory for those catalysts like the tin systems, if the added compound can occupy multiple sites on the tin it can be rendered inactive for polymerization (and depolymerization). For example, a compound like EDTA can occupy several sites in the coordination sphere of the tin and, in turn, interfere with the catalytic sites in the coordination sphere. Alternatively, the added compound can be of sufficient size and the catalyst can adhere to its surface, such that the absorbed catalyst may be filtered from the polymer. Those added compounds such as silica may have sufficient acidic/basic properties that the silica adsorbs the catalyst and is filterable.

The by-product of the amide polymerization product is water. A means to remove the water efficiently during the polymerization can be effective in obtaining (co)polymers with a high degree of conversion.

In a particular embodiment, a method of making poly amino-alpha, omega-dicarboxylic acid by the conversion of a crude aliphatic amino-alpha, omega-dicarboxylic acid monomer to a poly amino-alpha, omega-dicarboxylic acid, comprising the steps of:

a) providing a source of monomer as amino-alpha, omega-dicarboxylic acid in a hydroxylic medium;

b) concentrating the amino-alpha, omega-dicarboxylic acid in the hydroxylic medium by evaporating a substantial portion of the hydroxylic medium to form a concentrated acid solution;

c) oligomerizing the amino-alpha, omega-dicarboxylic acid to obtain an amino-alpha, omega-dicarboxylic acid oligomer;

d) adding a polymerization catalyst to the amino-alpha, omega-dicarboxylic acid oligomer;

e) polymerizing the amino-alpha, omega-dicarboxylic acid and amino-alpha, omega-dicarboxylic acid oligomer to obtain a poly amino-alpha, omega-dicarboxylic acid;

f) transferring the poly amino-alpha, omega-dicarboxylic acid to a thin film polymerization/devolatilization device;

g) isolating the poly amino-alpha, omega-dicarboxylic acid.

The thin film polymerization/devolatilization device is configured such that fluid polymer is conveyed to the device such that the film of the fluid polymer is less than 1 cm thick and provides a means for volatilizing the water formed in the reaction and other volatile components. The temperature of the thin film evaporator and polymerization/devolatilization device are from 100 to 240° C. and the pressure of the device is from 0.000014 to 50 kPa. A full vacuum may be used in the evaporator device. Pressures can be e.g., less than 0.01 torr, alternatively less than 0.001 torr and optionally less than 0.0001 torr.

The polymerization steps c, e, and f are three polymerization stages, 1, 2 and 3, of polymerization of the amino, dicarboxylic acid.

The thin film evaporator or thin film polymerization/devolatilization device are also a convenient place to add other components to the poly amino, dicarboxylic acid. These other components can include other monomers including the other amino, dicarboxylic acid, homologues of the amino, dicarboxylic acid, diols, hydroxy dicarboxylic acids, dicarboxylic acids, alcohol amines, diamines and similar reactive species. Reactive components such as peroxides, glycidyl acrylates, epoxides and the like can also be added at this stage in the process.

An extruder also can be in fluid contact or fluid communications with the thin film evaporator and/or thin film polymerization/devolatilization device and can be used to recycle the polymer and/or to provide the means to process the poly amino-carboxylic acid to the isolation portion of the process. The extruder is also a convenient device to add other components and reactives listed above and discussed below, especially if they would be volatilized in the thin film polymerization/devolatilization device. In one aspect, the disclosure relates to a method for making a product including treating a reduced recalcitrance biomass (e.g., lignocellulosic or cellulosic material) with one or more enzymes and/or organisms to produce amino-alpha, omega-dicarboxylic acid and converting the amino-alpha, omega-dicarboxylic acid to the product.

Optionally, when the polymerization method is direct condensation, the polymerization can include utilizing coupling agents and/or chain extenders to increase the molecular weight of the polymer. For example, the coupling agents and/or chain extenders can include triphosgene, carbonyl diimidazole, dicyclohexylcarbodiimide, diisocyanate, acid chlorides, acid anhydrides, epoxides, thiirane, oxazoline, orthoester, and mixtures of these. Alternatively, the polymer can have a co monomer which is a polycarboxylic acid polyamide or polyamines or a combination of these.

In the implementations wherein polymers are made from the aspartic or glutamic acid, the methods can further include blending the polymer with a second polymer. For example, a second polymer can include polyglycols, polyvinyl acetate, polyolefins, styrenic resins, polyacetals, poly(meth)acrylates, polycarbonate, polybutylene succinate, elastomers, polyurethanes, natural rubber, polybutadiene, neoprene, silicone, and combinations of these.

In other implementations wherein polymers are made from the aspartic or glutamic acid a co-monomer can be co-polymerized with the glutamic or aspartic acid or a lactide such as the lactide based on lactic acid. For example, the co-monomer can include elastomeric units, lactones, glycolic acid, carbonates, morpholinediones, epoxides, 1,4-benzodioxepin-2,5-(3H)-dione glycosalicylide, 1,4-benzodioxepin-2,5-(3H,3-methyl)-dione lactosalicylide, dibenzo-1,5 dioxacin-6-12-dione disalicylide, morpholine-2,5-dione, 1,4-dioxane-2,5-dione glycolide, oxepane-2-one E-caprolactone, 1,3-dioxane-2-one trimethylene carconate, 2,2-dimethyltrimethylene carbonate, 1,5-dioxepane-2-one, 1,4-dioxane-2-one p-dioxanone, gamma-butyrolactone, beta-butyrolactone, beta-me-delta-valerolactone, 1,4-dioxane-2,3-dione ethylene oxalate, 3-[benzyloxycarbonyl methyl]-1,4-dioxane-2,5-dione, ethylene oxide, propylene oxide, 5,5′(oxepane-2-one), 2,4,7,9-tetraoxa-spiro[5,5]undecane-3,8-dione Spiro-bid-dimethylene carbonate and mixtures of these.

In any implementation wherein polymers are made, the polymers can be combined with fillers (e.g., by extrusion and/or compression molding). For example, some fillers that can be used include silicates, layered silicates, polymer and organically modified layered silicate, synthetic mica, carbon, carbon fibers, glass fibers, boric acid, talc, montmorillonite, clay, starch, corn starch, wheat starch, cellulose fibers, paper, rayon, non-woven fibers, wood flours, whiskers of potassium titanate, whiskers of aluminum borate, 4,4′-thiodiphenol, glycerol and mixtures of these.

In any implementation wherein polymers are made, the method can further include branching and/or cross linking the polymer. For example, the polymers can be treated with a cross linking agent including 5,5′-bis(oxepane-2-one)(bis-ε-caprolactone)), spiro-bis-dimethylene carbonate, peroxides, dicumyl peroxide, benzoyl peroxide, unsaturated alcohols, hydroxyethyl methacrylate, 2-butene-1,4-diol, unsaturated anhydrides, maleic anhydride, saturated epoxides, glycidyl methacrylate, irradiation and combinations of these. Optionally, a molecule (e.g., a polymer) can be grafted to the polymer. For example, grafting can be done treating the polymer with irradiation, peroxide, crossing agents, oxidants, heating or any method that can generate a cationic, anionic or radicle on the polymer.

In any implementation wherein polymers are processed, processing can include injection molding, blow molding and thermoforming.

In any implementation wherein polymers are processed, the polymers can be combined with a dye and/or a fragrance. For example, dyes that can be used include blue3, blue 356, brown 1, orange 29, violet 26, violet 93, yellow 42, yellow 54, yellow 82 and combinations of these. Examples of fragrances include wood, evergreen, redwood, peppermint, cherry, strawberry, peach, lime, spearmint, cinnamon, anise, basil, bergamot, black pepper, camphor, chamomile, citronella, eucalyptus, pine, fir, geranium, ginger, grapefruit, jasmine, juniper berry, lavender, lemon, mandarin, marjoram, musk, myrrh, orange, patchouli, rose, rosemary, sage, sandalwood, tea tree, thyme, wintergreen, ylang ylang, vanilla, new car or mixtures of these fragrances. Fragrances can be used in any amount, for example, between about 0.005% by weight and about 20% by weight (e.g., between about 0.1% and about 5 wt. %, between about 0.25 wt. % and about 2.5%).

In any implementation wherein polymers are processed, the polymer can be blended with a plasticizer. For example, plasticizers include triacetin, tributyl citrate, polyethylene glycol, GRINDSTED® SOFT-N-SAFE (from Danisco, DuPont, Wilmington Del., diethyl bishydroxymethyl malonate) and mixtures of these.

In any of the implementations wherein polymers are made, the polymers can be processed or further processed by shaping, molding, carving, extruding and/or assembling the polymer into the product.

In another aspect, the invention relates to products made by the methods discussed above. For example, the products include a converted amino-alpha, omega-dicarboxylic acid wherein the amino-alpha, omega-dicarboxylic acids is produced by the fermentation of biomass derived sugars (e.g., aspartic acid, glutamic acid and the amino substituted malonic, adipic, pimelic, suberic azelaic and sebacic acids). The biomass includes cellulosic and lignocellulosic materials and these can release sugars by acidic or enzymatic saccharification. In addition, the biomass can be treated, e.g., by irradiation. The products, for example include polymers, including one or more amino dicarboxylic acids in the polymer backbone and optionally non-amino-alpha, omega-dicarboxylic acids in the polymer backbone. Optionally, the polymers can be cross-linked or graft co-polymers. Additionally, the polymer can be, blended with a second polymer, blended with a plasticizer, blended with an elastomer, blended with a fragrance, blended with a dye, blended with a pigment, blended with a filler or blended with a combination of these.

In yet another embodiment, the invention relates to a system for polymerization including a reaction vessel, a screw extruder and a condenser. The system also includes a recirculating fluid flow path from an outlet of the reaction vessel to an inlet of the screw extruder and from an outlet of the screw extruder to an inlet to the reaction vessel. In addition, the system includes a fluid flow path from a second outlet of the reaction vessel to an inlet of the condenser. Optionally, the system further includes a vacuum pump in fluid connection with the second fluid flow path for producing a vacuum in the second fluid flow path. Also optionally, the system can include a control valve that in a first position provides a non-disrupted flow in the recirculating fluid flow path and in a second position provides a second fluid flow path. In some implementations, the second fluid flow path is from the outlet of the reaction vessel to an inlet of a pelletizer. In other implementations the second fluid flow path is from the outlet of the reaction vessel to the inlet of the extruder and from the outlet of the extruder to the inlet of a pelletizer.

Some of the products described herein, for example, aspartic or glutamic acid, can be produced by chemical methods. However, fermentative methods can be much more efficient, providing high biomass conversion, selective conversion and high production rates. In particular, fermentative methods can produce D- or L-isomers or mixtures of amino-alpha, omega-dicarboxylic acids (e.g., aspartic or glutamic acid) at chiral purity of near 100% or mixtures of these isomers, whereas the chemical methods typically produce racemic mixtures of the D- and L-isomers. When an amino dicarboxylic acid is listed without its stereochemistry it is understood that D-, L-, meso, and/or mixtures are assumed.

The methods describe herein are also advantageous in that the starting materials (e.g., sugars) can be completely derived from biomass (e.g., cellulosic and lignocellulosic materials). In addition, some of the products described herein such as polymers of amino-alpha, omega-dicarboxylic acids (e.g., polyaspartic or polyglutamic acid) are compostable, biodegradable and/or recyclable. Therefore, the methods described herein can provide useful materials and products from renewable sources (e.g., biomass) wherein the products themselves can be re-utilized or simply safely returned to the environment.

For example, some products that can be made by the methods, systems or equipment described herein include personal care items, tissues, towels, diapers, green packaging, compostable pots, consumer electronics, laptop casings, mobile phone casings, appliances, food packaging, disposable packaging, food containers, drink bottles, garbage bags, waste compostable bags, mulch films, controlled release matrices, controlled release containers, containers for fertilizers, containers for pesticides, containers for herbicides, containers for nutrients, containers for pharmaceuticals, containers for flavoring agents, containers for foods, shopping bags, general purpose film, high heat film, heat seal layer, surface coating, disposable tableware, plates, cups, forks, knives, spoons, sporks, bowls, automotive parts, panels, fabrics, under hood covers, carpet fibers, clothing fibers, fibers for garments, fibers for sportswear, fibers for footwear, surgical sutures, implants, scaffolding and drug delivery systems.

Some of the products described herein, for example, glutamic acid or aspartic acid, can be produced by chemical methods. However, fermentative methods can be much more efficient, providing high biomass conversion, selective conversion and high production rates. In particular, fermentative methods can produce D- or L-isomers of amino-alpha, omega-dicarboxylic acids (e.g., glutamic acid and aspartic acid) at chiral purity of near 100% or mixtures of these isomers, whereas the typical chemical methods can typically produce racemic mixtures. The methods describe herein are also advantageous in that the starting materials (e.g., sugars) can be completely derived from biomass (e.g., cellulosic and lignocellulosic materials). In addition, some of the products described herein such as polymers of amino-alpha, omega-dicarboxylic acids (e.g., polyglutamic acid or polyaspartic acid) are compostable, biodegradable and/or recyclable. Therefore, the methods described herein can provide useful materials and products from renewable sources (e.g., biomass) wherein the products themselves can be re-utilized or simply safely returned to the environment. The amino-alpha, omega dicarboxylic acids can include 2-amino derivatives of malonic, adipic, pimelic, suberic azelaic sebacic, and substituted derivatives thereof. A generalized structure of the amino-alpha, omega-dicarboxylic acids is shown. The omega denotes the last carbon in the carbon chain.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, Appendices, patent applications, patents, and other references mentioned herein or attached hereto are incorporated by reference in their entirety for all that they contain. In case of conflict, the present specification, including definitions, will control. In addition, the materials, methods, and examples are illustrative only and not intended to be limiting. Other features and advantages of the invention will be apparent from the following detailed description, and from the claims.

### DESCRIPTION OF THE FIGURES

The foregoing will be apparent from the following more particular description of example embodiments of the invention, as illustrated in the accompanying. The drawings are not necessarily to scale, emphasis instead being placed upon illustrating embodiments of the present invention.

FIG. 1 is a flow diagram showing processes for manufacturing products from a biomass feedstock

FIG. 2 is a schematic view of a reaction system for polymerizing glutamic acid or aspartic acid.

FIG. 3A is a top view of a first embodiment of a reciprocating scraper. FIG. 3B is a front cut-out view of the first embodiment of a reciprocating scraper. FIG. 3C is a top view of a second embodiment of a reciprocating scraper. FIG. 3D is a front cut-out view of the second embodiment of a reciprocating scraper.

FIG. 4 shows four stereochemistry types for the polyamide of the amino-alpha, omega-dicarboxylic acids.

FIG. 5 shows pathways to form polyaspartic acid (PASA).

FIG. 6 shows a schematic of a polymerization system.

FIG. 7 shows a cutaway of the thin film polymerization/devolatilization device

FIG. 8 shows a schematic of a pilot-scale polymerization system.

FIG. 9 shows a cutaway of the thin film polymerization/devolatilization

### DESCRIPTION

Using the equipment, methods and systems described herein, cellulosic and lignocellulosic feedstock materials, for example, that can be sourced from biomass (e.g., plant biomass, animal biomass, paper, and municipal waste biomass) and that are often readily available but difficult to process, can be turned into useful products such as sugars and amino-alpha, omega-dicarboxylic acids. Included are equipment, methods and systems to chemically convert the primary products produced from the biomass to secondary product such as oligomers, polymers (e.g., homo and hetero polyglutamic and polyaspartic acid) and polymer derivatives (e.g., composites, elastomers, and co-polymers). An amino-alpha, omega-dicarboxylic acid is shown in Structure I with the amino group substituted at the 2-carbon. The omega denotes the last carbon in the carbon chain not including the carbon of the carboxylic acid group.

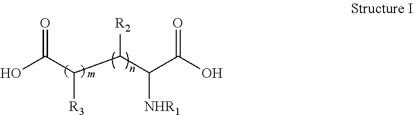

Where n and m are integers,

n=0 to 7,

m=0 to 7,

n+m≦10,

R₁=H, straight chain, branched alkyls, aromatics, or substituted alkyl aromatics with less than 24 carbons,

R₂=H, straight chain, branched alkyls, or substituted alkyl aromatics with less than 24 carbons,

R₃=H, NHR₁, straight chain, or substituted alkyl aromatics with less than 24 carbons.

In a particularly a preferred embodiment m=1 and n=1 and R₁, R₂, and R₃ are all hydrogen resulting in D-aspartic or L-aspartic acid shown in Structures Ia and Ib, respectively.

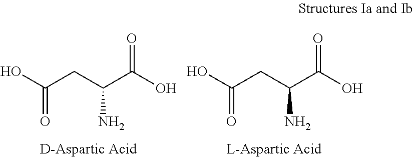

In a particularly a preferred embodiment m=1 and n=1 and R₁, R₂, and R₃ are all hydrogen resulting in D-glutamic acid or L-glutamic shown in Structures Ic and Id respectively.

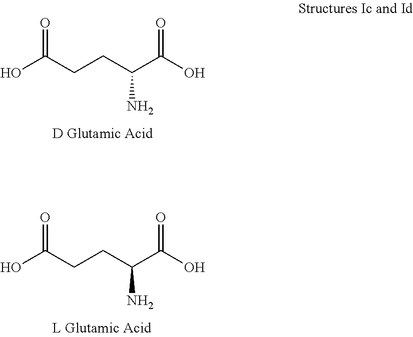 [SMILES: N[C@H](CCC(=O)O)C(=O)O ; N[C@@H](CCC(=O)O)C(=O)O]

Alternatively, the amino group can be substituted in other positions in the carbon chain. The amino-alpha, omega dicarboxylic acid with the amino group substituted at least one group removed from the carboxylic acid is shown in Structure II

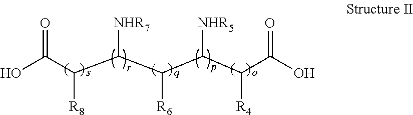

Where o, p, q, r and s are integers

o=1, 2, or 3;

p=1 or 2;

q=0, 1, 2, 3;

r=0, 1;

s=1, 2, or 3;

o+p+q+r+s≦10

R₄=H, straight chain, branched alkyls with less than 24 carbons, aromatics, or substituted alkyl aromatics,

R₅=H, straight chain, branched alkyls with less than 24 carbons, aromatics, or substituted alkyl aromatics,

R₆=H, straight chain, branched alkyls with less than 24 carbons, aromatics, or substituted alkyl aromatics,

R₇=H, straight chain, branched alkyls with less than 24 carbons, aromatics, or substituted alkyl aromatics,

R₈=H, straight chain, branched alkyls with less than 24 carbons, or substituted alkyl aromatics.

For example, in Structure I, m is chosen from 0, 1, 2, 3, 4, 5, 6, or 7; n is chosen from 0, 1, 2, 3, 4, 5, 6, or 7; with the limitation that n+m must be less than or equal to 10. and R₁, R₂, R₃ are chosen from hydrogen, straight chain or branched alkyl groups, aromatics, and alkyl aromatics where the limitation is that there are less than 24 carbons. Any combination of n, m and the R groups can be included in the alpha-amino, omega-dicarboxylic acid.

The alkyl groups, aromatic groups, and alkyl aromatic groups for R₁, R₂, R₃, R₄, R₅, R₆, R₇, and R₈, can be straight chain and may include methyl, ethyl, propyl, butyl, pentyl, hexyl, heptyl, octyl, nonyl, decyl, lauryl, myristic, palmitic, stearic, arachidic, behenic, up to and including a 24 carbons. The branched chain may include, isopropyl, 2-butanyl, fsa2 and 3-pentyl, 2, 3, and 4 hexyl and other branched hydrocarbons up to 24 carbons. The alkyl aromatic may include alkyl substituted benzene, alkyl substituted naphthalene, and similar substituted alkyl aromatic compounds.

The amino-alpha, omega-dicarboxylic acid exists in various forms depending on the pH of its environment. It is understood that the amino-alpha, omega-dicarboxylic acid is meant to include all of these pH dependent forms. For example, for glutamic acid, the pKa₁=-carboxyl group, pKa₂=α-ammonium ion, and pKa₃=side chain group as the omega carboxylic acid, are 2.19, 9.67, 4.25 respectively and the isoelectronic point is 3.22. As with all amino acids, the presence of acid protons depends on the residue's local chemical environment and the pH of the solution. The amphiphilic nature of these compounds lead to useful and varied products.

Biomass is a complex feedstock. For example, lignocellulosic materials include different combinations of cellulose, hemicellulose and lignin. Cellulose is a linear polymer of glucose. Hemicellulose is any of several heteropolymers, such as xylan, glucuronoxylan, arabinoxylans and xyloglucan. The primary sugar monomer present (e.g., present in the largest concentration) in hemicellulose is xylose, although other monomers such as mannose, galactose, rhamnose, arabinose and glucose are present. Although all lignins show variation in their composition, they have been described as an amorphous dendritic network polymer of phenyl propene units. The amounts of cellulose, hemicellulose and lignin in a specific biomass material depend on the source of the biomass material. For example, wood-derived biomass can be about 38-49% cellulose, 7-26% hemicellulose and 23-34% lignin depending on the type. Grasses typically are 33-38% cellulose, 24-32% hemicellulose and 17-22% lignin. Clearly lignocellulosic biomass constitutes a large class of substrates.

Enzymes and biomass-destroying organisms that break down biomass, such as the cellulose, hemicellulose and/or the lignin portions of the biomass as described above, contain or manufacture various cellulolytic enzymes (cellulases), ligninases, xylanases, hemicellulases or various small molecule biomass-destroying metabolites. A cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally cellobiase cleaves cellobiose to yield glucose. In the case of hemicellulose, a xylanase (e.g., hemicellulase) acts on this biopolymer and releases xylose as one of the possible products.

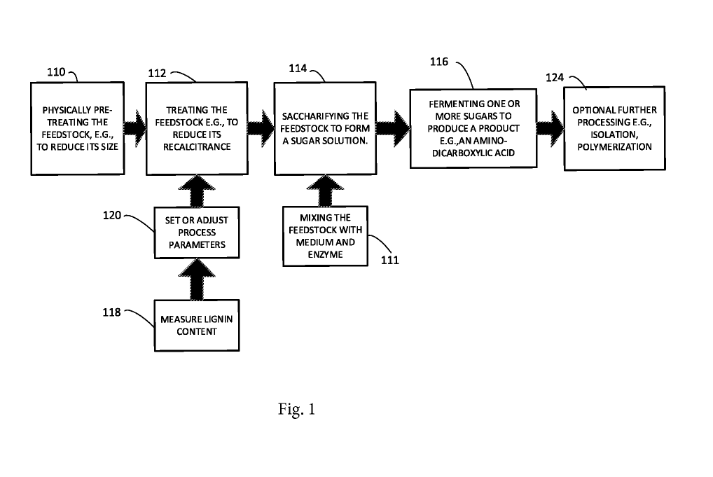

FIG. 1 is a flow diagram showing processes for manufacturing is a flow diagram showing processes for manufacturing amino-alpha, omega-dicarboxylic acids from a feedstock (e.g., cellulosic or lignocellulosic materials). In an initial step (110) the method includes optionally mechanically treating a cellulosic and/or lignocellulosic feedstock. Before and/or after this treatment, the feedstock can be treated with another physical treatment (112), for example, irradiation, sonication, size reduction, steam explosion, oxidation, pyrolysis or combinations of these, to reduce or further reduce its recalcitrance. A sugar solution e.g., including glucose and/or xylose, is formed by saccharifying the feedstock (114). The saccharification can be, for example, accomplished efficiently by the addition of one or more enzymes, e.g., cellulases and/or xylanases (111). A product or several products can be derived from the sugar solution, for example, by fermentation to amino-alpha, omega-dicarboxylic acids (116). Following fermentation, the fermentation product (e.g., or products, or a subset of the fermentation products) can be purified or further processed, for example polymerized and/or isolated (124). Optionally, the sugar solution is a mixture of sugars and the organism selectively ferments only one of the sugars. The fermentation of only one of the sugars in a mixture can be advantageous as described in International App. No. PCT/US2014/021813 filed Mar. 7, 2014, the entire disclosure of which is incorporated herein by reference. If desired, the steps of measuring lignin content (118) and setting or adjusting process parameters based on this measurement (120) can be performed at various stages of the process, for example, as described in U.S. Pat. No. 8,415,122, issued Apr. 9, 2013 the entire disclosure of which is incorporated herein by reference. Optionally, enzymes (e.g., in addition to cellulases and xylanases) can be added in step (114), for example, a glucose isomerase can be used to isomerize glucose to fructose. Some relevant uses of isomerase are discussed in PCT Application No. PCT/US12/71093, filed on Dec. 20, 2012, the entire disclosure of which is incorporated herein by reference.

In some embodiments the liquids after saccharification and/or fermentation can be treated to remove solids, for example, by centrifugation, filtration, screening, or rotary vacuum filtration. For example, some methods and equipment that can be used during or after saccharification are disclosed in International App. No. PCT/US2013/048963 filed Jul. 1, 2013, and International App. No. PCT/US2014/021584, filed on Mar. 7, 2014, the entire disclosures of which are incorporated herein by reference. In addition, other separation techniques can be used on the liquids, for example, to remove ions and de-colorize. For example, chromatography, simulated moving bed chromatograph and electrodialysis can be used to purify any of the solutions and or suspensions described herein.

Some of these methods are discussed in International App. No. PCT/US2014/021638, filed on Mar. 7, 2014, and International App. No. PCT/US2014/021815, filed on Mar. 7, 2014, the entire disclosures of which are incorporated herein by reference. Solids that are removed during the processing can be utilized for energy co-generation, for example, as discussed in International App. No. PCT/US2014/021634, filed on Mar. 7, 2014, the entire disclosure of which is herein incorporated by reference.

Optionally the sugars released from biomass as describe in FIG. 1, for example glucose, xylose, sucrose, maltose, lactose, mannose, galactose, arabinose, dimers (e.g., cellobiose, sucrose), trimers, oligomers and mixtures of these, can be fermented to amino-alpha, omega-dicarboxylic acids. In some embodiments the saccharification and fermentation are done simultaneously.

### Preparation of Amino-Alpha, Omega Dicarboxylic Acid

Organisms can utilize a variety of metabolic pathways to convert the sugars to amino-alpha, omega-dicarboxylic acids, and some organisms selectively only can use specific pathways. Some organisms are homofermentative while others are heterofermentative.

Using the methods, equipment and systems described herein, either D- or L-isomers of aspartic acid at an optical purity of near 100% (e.g., at least about 80%, at least about 85%, at least about 90%, at least about 95%, at least about 99%) can be produced. Optionally mixtures of the isomers can be produced in any ratio, for example, from 0% optical purity of any isomer up to 100% optical purity of any isomer. For example, genetically modified organisms can also be utilized.

Co-cultures of organisms, for example chosen from organisms as describe herein, can be used in the fermentations of sugars to amino-alpha, omega-dicarboxylic acids in any combination. For example, two or more bacteria, yeasts and/or fungi can be combined with one or more sugars (e.g., glucose and/or xylose) where the organisms ferment the sugars together, selectively and/or sequentially. Optionally, one organism is added first and the fermentation proceed for a time, for example, until it stops fermenting one or more of the sugars, and then a second organism can be added to further ferment the same sugar or ferment a different sugar. Co-cultures can also be utilized, for example, to tune in a desirable racemic mixture of D- and L-aspartic acid by combining a D-fermenting and L-fermenting organism in an appropriate ratio to form the targeted mixture of stereoisomers. Co-cultures can also be utilized to prepare mixtures of amino-alpha, omega-dicarboxylic acids, specifically, aspartic and glutamic acid in such a ratio that can lead to a copolymer of the aspartic and glutamic acids in a desired ratio.

In some embodiments some additives (e.g., media components) can be added during the fermentation. For example, additives that can be utilized include yeast extract, rice bran, wheat bran, corn steep liquor, black strap molasses, casein hydrolyzate, vegetable extracts, corn steep solid, ram horn waste, peptides, peptone (e.g., bactopeptone, polypeptone), pharmamedia, flower (e.g., wheat flour, soybean flour, cottonseed flour), malt extract, beef extract, tryptone, K₂HPO₄, KH₂PO₄, Na₂HPO₄, NaH₂PO₄, (NH₄)₂PO₄, NH₄OH, NH₄NO, urea ammonium citrate, nitrilotriacetic acid, MnSO₄.5H₂O, MgSO4.7H₂O, CaCl₂.2H₂O, FeSO₄.7H₂O, B-vitamins (e.g., thiamine, riboflavin, niacin, niacinamide, pantothenic acid, pyridoxine, pyridoxal, pyridoxamine, pyridoxine hydrochloride, biotin, folic acid), amino acids, sodium-L-glutamate, Na₂EDTA, sodium acetate, ZnSO4.7H₂O, ammonium molybdate tetrahydrate, CuCl₂, CoCl₂ and CaCO₃. Addition of protease can also be beneficial during the fermentation. Optionally, surfactants such as TWEEN™80 and antibiotics such as penicillin and chloramphenicol can also be beneficial. Additional carbon sources, for example glucose, xylose and other sugars. Antifoaming compounds such as Antifoam 204 can also be utilized.

In some embodiments the fermentation can take from about 8 hours to several days. For example, some batch fermentations can take from about 1 to about 20 days (e.g., about 1-10 days, about 3-6 days, about 8 hours to 48 hours, about 8 hours to 24 hours).

In some embodiments the temperature during the fermentation is controlled. For example, the temperature can be controlled between about 20° C. and 50° C. (e.g., between about 25 and 40° C., between about 30 and 40° C., between about 35 and 40° C.). In some case thermophilic organisms are utilized that operate efficiently above about 50° C., for example, between about 50° C. and 100° C. (e.g., between about 50-90° C., between about 50 to 80° C., between about 50 to 70° C.).

In some embodiments the pH is controlled, for example, by the addition of an acid or a base. The pH can be optionally controlled to be close to neutral (e.g., between about 4-8, between about 5-7, between about 5-6). Acids, for example, can be protic acids such as sulfuric, phosphoric, nitric, hydrochloride and acetic acids. Bases, for example, can include metal hydroxides (e.g., sodium and potassium hydroxide), ammonium hydroxide, and calcium carbonate. Phosphate and other buffers can also be utilized.

Several organisms can be utilized to ferment the biomass derived sugars to amino-alpha, omega-dicarboxylic acids. The organisms can be, for example, Corynebacterium, Corynebacterium glutamicum, bacillus, Lactobacillus arabinosus e. coli, Rhizobium japonicum, Brevibacterium flavum AJ 3859, Brevibacterium lactofermentum AJ 3860, Corynebacterium acetoacidophilum, Corynebacterium glutamicum (Micrococcus glutamicus), Serratia marcescens, Pseudomonas fluorescens, Protens vulgaris, Pseudomonas aeruginosa, Bacterium succinium, Bacillus subtilis, Aerobacter aerogenes, Micrococcus sp., Escherichia coli, Rhizobium lupini bacteroides, genetically modified organisms and the like. Blends of microorganisms may be needed so that the sugar is converted to a substrate that the amino, omega dicarboxylic acid producing microorganism may use.

The organism described above may also need a nitrogen source, which include ammonia, ammonium salts, urea and the like. Alternately, a fermentation microorganism (described below) can be combined with enzymes such as N-acetyl-glutamate synthase, transaminase, glutaminase, (an amidohydrolase enzyme), glutamate dehydrogenase, aldehyde dehydrogenase, formiminotransferase cyclodeaminase, glutamate carboxypeptidase II, and the like to produce amino, dicarboxylic acids.

Fermentation methods include, for example, batch, fed batch, repeated batch or continuous reactors. Often batch methods can produce higher concentrations of amino-alpha, omega-dicarboxylic acid, while continuous methods can lead to higher productivities.

Fed batch methods can include adding media components and substrate (e.g., sugars from biomass) as they are depleted. Optionally, products, intermediates, side products and/or waste products can be removed as they are produced. In addition, solvent (e.g., water) can be added or removed to maintain the optimal amount for the fermentation.

Options include cell-recycling. For example, using a hollow fiber membrane to separate cells from media components and products after fermentation is complete. The cells can then be re-utilized in repeated batches. In other optional methods the cells can be supported, for example, as described in U.S. application Ser. No. 13/293,971, filed on Nov. 10, 2011 and U.S. Pat. No. 8,377,668, issued Feb. 19, 2013 the entire disclosures of which are herein incorporated by reference.

The fermentation broth can be neutralized using calcium carbonate or calcium hydroxide which can form the calcium salts of the amino-alpha, omega-dicarboxylic acids. The mono or di calcium amino-alpha, omega-dicarboxylic acids broth can then be filtered to remove cells and other insoluble materials. In addition, the broth can be treated with a decolorizing agent. For example, the broth can be filtered through carbon. The broth is then concentrated, e.g., by evaporation of the water optionally under vacuum and/or mild heating, and can be crystallized or precipitated. Acidification, for example, with sulfuric acid, releases the amino-alpha, omega-dicarboxylic acids acid back into solution which can be separated (e.g., filtered) from the insoluble calcium salts, e.g., calcium sulfate. Addition of calcium carbonate during the fermentation can also serve as a way to reduce product inhibition since the calcium amino-alpha, omega-dicarboxylic acids is not inhibitory or causes less product inhibition.

Other metal salts can be used. When the amino group is substituted at the 2 position the 2-amino-alpha, omega-dicarboxylic acids can form a metal chelate that can be isolated. Following isolation the 2-amino-alpha, omega-dicarboxylic acid chelate can be converted back to the 2-amino-alpha, omega-dicarboxylic acid and the metal salt facilitating isolation of the 2-amino-alpha, omega-dicarboxylic acid.

Optionally, reactive distillation may be used to purify amino-alpha, omega-dicarboxylic acids. For example, methylation of an amino-alpha, omega-dicarboxylic acid provides the dimethyl and/or the methyl ester which can be distillated to pure ester which can then be hydrolyzed to the diacid and methanol that can be recycled. Esterification to other esters can also be used to facilitate the separation. For example, reactions with alcohols to the ethyl, propyl, butyl, hexyl, octyl or even esters with more than eight carbons can be formed and then extracted in a solvent or distilled.

Other alternative amino-alpha, omega-dicarboxylic acids separation technologies include adsorption, for example, on activated carbon, polyvinylpyridine, zeolite molecular sieves and ion exchange resins such as basic resins. Other methods include ultrafiltration, transition recrystallization, and electrodialysis(including using two compartment bipolar membranes).

Precipitation or crystallization of calcium amino-alpha, omega-dicarboxylic acid by the addition of organic solvents is another method for purification. For example, alcohols (e.g., ethanol, propanol, butanol, hexanol), ketones (e.g., acetone) can be utilized for this purpose. Other metal salts, especially those that form a chelate may be crystallization with these alternative solvent.

Glutamic acid: For example, several fermentation pathways are known that make glutamic acid. These pathways include hydrolysis of glutamine or N-acetyl glutamic acid; transamination of α-ketoglutarate; dehydrogenation of α-ketoglutarate; dehydrogenation of 1-pyrroline-5-carboxylate by 1-pyrroline-5-carboxylate dehydrogenase; and other known fermentation pathways.

Corynebacterium glutamicum is especially useful for the production of glutamic acid. Genetically modified organisms can also be utilized to produce the amino-alpha, omega-dicarboxylic acid.

Aspartic acid: For example, several fermentation pathways are known that make aspartic acid. L-aspartic acid can be continuously produced from fumarate and ammonia with immobilized E. coli cells.

Similar methods can be utilized for the preparation of other amino-dicarboxylic acids. For example, the fermentative methods and procedures can be applicable for any of the amino-dicarboxylic acids described herein.

Products Derived from Amino-Alpha, Omega-Dicarboxylic Acid

Amino-alpha, omega-dicarboxylic acid produced as described herein can be used, for a variety of purposes. Its uses are derived from the structural aspects in that there is at least one amino group and two dicarboxylic acid groups. Each of these three groups has different chemistry associated with it. The amino group and either of the carboxylic acids can cyclize to form a four, five six or seven member lactam ring which can undergo further reaction. These cyclic compounds refer to the aspartic acid, glutamic acid, 2-aminoadipic acid and 2 amino pimelic acid respectively. The amino group and carboxylic acid can also form a chelate about a metal ion. The amphiphilic properties of these amino-alpha, omega-dicarboxylic acids provide a broad range of properties, especially in aqueous systems.

Many polymerization products can be made. One example, is the polyamide which results in a carboxylic acid being pendant to the polymer backbone. This polyamide is like a polyacrylate with a heteroatom backbone and as such may have more hydrophilic properties relative to the polyacrylates. Polyacrylates are not biodegradable, but these polyamides can be biodegradable. Another polymerization can result in a polyester or polyamide with polymerization utilizing the alpha and omega carboxylic acids and a diol or diamine respectively. These polymerizations may require using protecting groups for the unused reacting group. If the polymerization utilizes the carboxylic acid and the amine that is substituted on the same carbon as the amine it is described as an alpha product. If the carboxylic acid is the omega carboxylic acid polymer can be described by the carboxylic acid position of the chain. For aspartic acid the carboxylic acid is beta and for glutamic acid the carboxylic acid is gamma. The polymers may be homopolymers, copolymers with different amino-alpha, omega-dicarboxylic acid and copolymers with other monomers.

Products from amino-alpha, omega-dicarboxylic acid and their polymeric products include flavor enhancers, nutrients, plant growth additives, dispersants, adhesives, water softener chemicals, waste water treatment, water treatment, a component in food and cosmetics, as a superabsorbent (hydrogels), humectant, components in coatings, treatment of leather, drug delivery systems. In biological systems the amino-alpha, omega-dicarboxylic acids are useful in metabolism, as a gamma-amino butyric acid precursor, a neurotransmitter and a brain no synaptic glutamatergic signaling circuits.

The use of amino-alpha, omega dicarboxylic acids as dispersants offers interesting contrasts to dispersants such as poly (meth) acrylic dispersants. The dispersant use could be as an amide polymer or copolymer of amino-alpha, omega-dicarboxylic acids. The pendant carboxylic acid can act as the water compatible group in the dispersant. Since the polymer should be biodegradable it should offer different advantages relative to poly (meth) acrylic dispersants which are not biodegradable. The polymer may be a random polymer or a structured polymer. Products from these amino-alpha, omega dicarboxylic acids include flavor enhancers, coatings, dispersants, superabsorbent, drug delivery systems, plant growth, metal chelator, waste water treatment, water treatment, automotive additives.

The biomass derived amino-alpha, omega-dicarboxylic acids as described herein can be used in pharmaceutical applications, for example, for pH-regulation, metal sequestration, as a chiral intermediate and as a natural body constituent in pharmaceutical products.

Products Derived from Aspartic Acid

An important amino-alpha, omega-dicarboxylic acids is aspartic acid. D-, L- and D-, L-aspartic acids may be utilized in many products. There are two forms or enantiomers of aspartic acid. The name “aspartic acid” can refer to either enantiomer or a mixture of two. Of these two forms, only one, “L-aspartic acid”, is directly incorporated into proteins. The biological roles of its counterpart, “D-aspartic acid” are more limited. Where enzymatic synthesis will produce one or the other, most chemical syntheses will produce both forms, “D-, L-aspartic acid,” known as a racemic mixture. Aspartic acid is non-essential in mammals, being produced from oxaloacetate by transamination. It can also be generated from ornithine and citrulline in the urea cycle. In plants and microorganisms, aspartate is the precursor to several amino acids, including four that are essential for humans: methionine, threonine, isoleucine, and lysine. The most prominent use of L-aspartic acid is its use in the sugar substitute aspartame. A dimer of aspartic acid D-, L-aspartic-N-(1,2-dicarboxyethyl)tetra sodium salt also known as sodium iminodisuccinate is used as a chelate for calcium to soften water and improve the cleaning function of the surfactant. Also, the chelating agent with metals can be used in agricultural applications to prevent, correct and minimize crop mineral deficiencies. Another simple derivative of aspartic acid is the reaction product of phosgene and similar reactants to produce the N-carboxyanhydride (NCA) derivatives. Many industrial uses are derived from polymers of aspartic acids which are described below.

Products Derived from Glutamic Acid

D-, L- and D-, L-glutamic acids may be utilized in many products. There are two forms or enantiomers of glutamic acid. The name “glutamic acid” can refer to either enantiomer or a mixture of two. Of these two forms, only one, “L-glutamic acid”, is directly incorporated into proteins. The biological roles of its counterpart, “D-glutamic acid” are more limited. Where enzymatic synthesis will produce one or the other, most chemical syntheses will produce both forms, “D-, L-glutamic acid,” known as a racemic mixture. Glutamic acid (abbreviated as Glu or E) is one of the 20-22 proteinogenic amino acids, and its codons are GAA and GAG. It is a non-essential amino acid. The carboxylate anions and salts of glutamic acid are known as glutamates.

The most prevalent industrial use of glutamic acid is as flavor additive in the mono sodium glutamate form, MSG. Another simple derivative of glutamic acid is the reaction product of phosgene and similar reactants to produce the N-carboxyanhydride (NCA) derivatives. Auxigro is a plant growth preparation that contains 30% glutamic acid. Emerging industrial uses are derived from polymers of glutamic acids which are described below.

### Polymerization of Amino-Alpha, Omega-Dicarboxylic Acids

Polymers of amino-alpha, omega-dicarboxylic acid are formed via many different polymerization schemes. Products include dimers, trimers, oligomers and polymers. One of these schemes results in a polyamide prepared as described herein can undergo an amide condensation to form polymers of amino-alpha, omega-dicarboxylic acid is a polyamide with the amide linkage at the alpha and/or omega carboxylic acid. For polyaspartic acid the amide polymer is a mixture of amide being formed with the alpha or beta carboxylic acid. For polyglutamic acid the amide polymer is a mixture of amide being formed with the alpha or gamma carboxylic acid. Polymers can also be made by polymerizing the NCA derivative of the amino-alpha, omega-dicarboxylic acid. The polymerization of amino-alpha, omega-dicarboxylic acids may be done with chemical means or via biochemical processes. The biochemical processes can lead to polymer products with stereo control, whereas the chemical processes will likely lead to racemized products.

Another example of a polymer product of amino-alpha, omega-dicarboxylic acids can be as a polyester via copolymerization with a diol. The amino group may need to be protected for this polymerization scheme to be viable. In a similar manner a different polyamide can be made if the amino-alpha, omega-dicarboxylic acid (possibly with the amino group protected) is copolymerized with a diamine.

A low molecular weight polymer of amino-alpha, omega-dicarboxylic acid can be produced can be made by controlling the reaction conditions. This method produces low molecular weight polymers. The condensation produces water which can prevent the production of high molecular weight polymers of amino-alpha, omega-dicarboxylic acid since the amide condensation reaction can be reversible. In addition, amide can be produced by backbiting from a chain end to form the lactam ring which reduces the molecular weight of the linear polymer.

One method for production of high molecular weight polymers of amino-alpha, omega-dicarboxylic acid is by coupling low Mw polymers of amino-alpha, omega-dicarboxylic acid, for example, made as described above, using chain coupling agents. For example, amine/carboxylic acid terminated polymers of amino-alpha, omega-dicarboxylic acid can be synthesized by the condensation of carboxylic acid in the presence of small amounts of multifunctional hydroxyl compounds such as, ethylene glycol, propylene glycol, 1,3-propanediol, 1,2-cyclohexanediol, 2-butene-1,4-diol, glycerol, 1.4-butanediol, 1,6-hexanediol. Alternatively, carboxyl-terminated polymers of amino-alpha, omega-dicarboxylic acid can be achieved by the condensation of amine functional group in the presence of small amounts of multifunctional carboxylic acids such as maleic, succinic, adipic, itaconic and malonic acid to form additional amide linkages. Other chain extending agents can have heterofunctional groups that couple either on the carboxylic acid end group of the PASA or the amino end group, for example, 6-hydroxycapric acid, mandelic acid, 4-hydroxybenzoic acid, 4-acetoxybenzoic acid. In a similar manner the amine end group may be reacted with diisocyanates which can form a urea linkage.

Esterification promotion agents can also be combined with aspartic acid to increase the molecular weight of polymers of amino-alpha, omega-dicarboxylic acid. For example, ester promotion agents include phosgene, diphosgene, triphosgene dicyclohexylcarbodiimide and carbonyldiimidazole. Some potentially undesirable side products can be produced by this method adding purification steps to the process. After final purification, the product can be very clean, free of catalysts and low molecular weight impurities.

The polymer molecular weights can also be increase by the addition of chain extending agents such as isocyanates, acid chlorides, anhydrides, epoxides, thiirande and oxazoline and orthoester.

Any of the polymerization schemes presented above can include small amounts of trisubsituted monomers such as a triamine, triol, a triisocyanate and the like to lead to some branching in the polymers. If too much trisubstituted monomer is included the polymers of amino-alpha, omega-dicarboxylic acid may polymerize into a highly cross-linked material.

The polymers of amino-alpha, omega-dicarboxylic acid may be polymerized with other monomers to form random or structured polymers. The structured polymers may include graft, block, star and other structured polymerization schemes.

Azeotropic condensation polymerization is another method to obtain high molecular weight polymer and does not require chain extenders or coupling agents. A general procedure for this route consists of reduced pressure (between 0.1-300 mm Hg) refluxing of polymerization of amino-alpha omega dicarboxylic acids for 1-10 hours between 110° C.-160° C. to remove majority of the condensation water. Catalyst and/or solvents are added and heated further for 1-10 hours between 110° C.-180° C. under 0.1-300 mm Hg. The polymer is then isolated or dissolved (methylene chloride, chloroform) and precipitated by the addition of a solvent (e.g., methyl ether, diethyl ether, methanol, ethanol, isopropanol, ethyl acetate, toluene) for further purification. Solvents used during to polymerization, catalyst, reaction time, temperature and level of impurities effect the rate of polymerization and hence the final molecular weight.

Additives, catalysts and promoters that can optionally be used include Protonic acids such as H₃PO₄, H₂SO₄, methane sulfonic acid, p-toluene sulfonic acid, supported sulfonic acid, NAFION® NR 50 H+ form From DuPont, Wilmington Del., Acids supported on polymers, Metal catalysts, for example, include Mg, Al, Ti, Zn, Sn. Some metal oxides that can optionally catalyze the reaction include TiO₂, ZnO, GeO₂, ZrO₂, SnO, SnO₂, Sb₂O₃. Metal halides, for example, that can be beneficial include ZnCl₂, SnCl₂, SnCl₄. Other metal containing catalysts that can optionally be used include Mn(AcO)₂, Fe₂(LA)₃, Co(AcO)₂, Ni(AcO)₂, Cu(OA)₂, Zn(LA)₂, Y(OA)₃, Al(i-PrO)₃, Ti(BuO)₄, TiO(acac)₂, (Bu)₂SnO, tin octoate. Combinations and mixtures of the above catalysts can also be used. For example, two or more catalysts can be added at one time or sequentially as the polymerization progresses. The catalysts can also be removed, replenished and or regenerated during the course of the polymerization are for repeated polymerizations. Some preferred combinations include protonic acids and one of the metal continuing catalysts, for example, SnCl₂/p-toluenesulfonic acid.

The azeotropic condensation can be done partially or entirely using a solvent. For example, a high boiling and aprotic solvent such as diphenyl ether, p-xylene, o-chlorotoluene, o-dichlorobenzene and/or isomers of these. The polymerization can also be done entirely or partially using melt polycondensation. Melt polycondensations are done above the melting point of the polymers/oligomers without organic solvents. For example, at the beginning of the polymerization when there is a high concentration of low molecular weight species (e.g., acid amino-alpha, omega-dicarboxylic acid and oligomers) there can be less need for a solvent, while as the molecular weight of the polymers increases, the addition of a high boiling solvent can improve the reaction rates.

During the polymerization, for example, especially at the beginning of the polymerization when the concentration of amino-alpha, omega-dicarboxylic acid is high and water is being formed at a high rate, the amino-alpha, omega-dicarboxylic acid, acid/water azeotropic mixture can be condensed and made to pass through molecular sieves to dehydrate the amino-alpha, omega-dicarboxylic acid which is then returned to the reaction vessel.

Since removal of water is essential for polymerization to the polyamide, a thin film polymerization/devolatilization device may be used to facility the polymerization while removing the water. In one embodiment, the method of making a high molecular weight polymer or copolymer from oligomer, the method comprising evaporating water as it is formed during condensation of an amino alpha, omega dicarboxylic acid oligomer e.g. an aliphatic amino alpha, omega dicarboxylic acid, as it traverses a surface of a thin film evaporator. Water, as a coproduct of the condensation, needs to be removed from the high molecular weight polymer or copolymer, to maximize the conversion to higher molecular weight materials and minimize the undesirable reverse reaction where the water adds back and releases a monomer unit, dimer unit or oligomer of the amino alpha, omega dicarboxylic acid.

In another embodiment, the thin film evaporator unit operation is described in more detail and the polymerization process is denoted in three steps or stages for the conversion to high molecular weight polymers. FIG. 1 shows a schematic of the polymerization process with the three polymerization steps indicated. A recycle loop which takes the product of the thin film evaporator/thin film polymerization/devolatilization device and recycle to the input of the thin film evaporator/thin film polymerization/devolatilization device. The thin film evaporator/thin film polymerization/devolatilization device product stream may be split between recycle and sending a portion of the product stream to product collection.

First, excess water is removed from the amino alpha, omega dicarboxylic acid. As the acid is derived from biomass processing, it is likely in an aqueous solvent. The condensation process to make the polyamide produces water and thus, the excess water must be removed. The removal may be done in a batch or continuous process, with or without vacuum and at temperatures to achieve effective water removal rates. Some condensation to form amide bonds can occur during step 1 and low molecular weight oligomers may be formed.

After excess water is removed, further heating and, optional processing with vacuum to remove more water. At this stage in the process, the conversion of the amino alpha, omega dicarboxylic acid results in an increased degree of oligomerization based on the number average molecular weight of the oligomer/polymer.

A polymerization catalyst is added to the oligomer/polymer system. The candidate polymer catalyst(s) is described above and further described below. The catalyst may be added by any convenient means. For instance, the catalyst components can be dissolved/dispersed into the amino alpha, omega dicarboxylic acid and added to the oligomer/polymer.

With the catalyst present more conversion of the oligomer/polymer mixture occurs to obtain a higher degree of polymerization. This is achieved by the catalytic action of the catalysts added and a combination of more heating and higher vacuums.

Next the thin film polymerization/devolatilization device can be utilized to obtain polymers with even higher a degree of polymerization. The thin film has a thickness of less than 1 cm, optionally less than 0.5 cm, or further less than 0.25 cm, additional less than 0.1 cm.

The thin film polymerization/devolatilization device is configured such that fluid polymer is conveyed to the device such that the film of the fluid polymer is less than 1 cm thick and the device provides a means for volatilizing the water formed in the reaction and other volatile components. The polymerization type is characterized as polymerization in the melt phase. The thin/film polymerization/devolatilization device and the thin film evaporator describe herein are similar in that they accomplish the same function.

The hydroxylic medium can be water; mixtures of water and compatible solvents such as methanol, ethanol; and low molecular weight alcohols such as methanol, ethanol, n-propanol, iso-propanol, n-butanol, iso-butanol, and similar alcohols.

The temperature of the thin film polymerization/devolatilization device is from 100 to 260° C. Optionally, the temperature of the thin film polymerization/devolatilization device is from 120 to 240° C. Additionally, the temperature is between 140 and 220° C.

The thin film polymerization/devolatilization device can operate at high vacuum with pressures of 0.0001 torr and lower. The thin film polymerization/devolatilization device can operate e.g. at 0.001 torr or lower; or 0.01 torr or lower. During some stages of operation the operating pressures can be considered low and medium vacuum; 760 to 25 torr and 25 to 0.001 torr, respectively. The device may operate at a pressure of 100 to 0.0001 torr, or alternatively, 50 to 0.001 torr, or optionally, 25 to 0.001 torr.

The thin film polymerization/devolatilization device can optionally include a recycle loop in which the melt polymerization product is recycled to the entrance point of the thin film polymerization/devolatilization device. It can also be coupled to an extrusion device. The melt polymerization product can be processed from the thin film polymerization/devolatilization device to an extruder which can pass the product to finished product area. Alternately, the flow of the output of the extruder may be directed back to the thin film polymerization/devolatilization device. The flow may be split between the product and the recycle. The extruder system is a convenient location to incorporate additives into the melt polymerization product for recycle and subsequent reaction or for blending into the polymer stream prior to transferring to the product finishing area. The additives were described above and below. The additive addition includes compounds that react into the polymer, react on the polymer or physically mix with the polymer.

Optionally, the catalyst may be removed from the molten polymer. Removing catalyst may be accomplished just prior to, during, or after the thin film evaporator/thin film polymerization/devolatilization device. The catalyst may be filtered from the molten polymer by using a filtration system similar to a screen pack. Since the molten polymer is flowing around the thin film evaporator/thin film polymerization/devolatilization device, a filtration system can be added.

To facilitate the catalyst removal a neutralization or chelation chemical may be added. Candidate compounds include phosphites, anhydrides, poly carboxylic acids, polyamines, hydrazides, EDTA (and similar compounds) and the like. These neutralization and/or chelation compounds can be insoluble in the molten polymer leading to facile filtration. Polycarboxylic acids include polyacrylic acids and polymethacrylic acids. The latter can be in a both a random, block, and graft polymer configuration. The amines include ethylene diamine, oligomers of ethylene diamine and other similar polyamines such as methyl bis-3-amino, propane.

Another option to remove the catalyst includes adding solid materials to the polymer melt. Examples of added materials include silica, alumina, aluminosilicates, clays, diatomaceous earth, polymers and like solid materials. Each of these can be optionally functionalized to react/bind with the catalyst. When the catalyst binds/bonds to these structures it can be filtered from the polymer.

Copolymers can be produced by adding monomers other than amino-alpha, omega-dicarboxylic acid during the azeotropic condensation reaction. For example, any of the multifunctional hydroxyl, carboxylic compounds or the heterofunctional compounds that can be used as coupling agents for low molecular weight polymers of amino-alpha, omega-dicarboxylic acid can also be used as co-monomers in the azeotropic condensation reaction.

Optionally, ring opening polymerization of the 5 member ring of amino-alpha, omega-dicarboxylic acid can provide polymers of amino-alpha, omega-dicarboxylic acid. Methods to form the polymers of amino-alpha, omega-dicarboxylic acid include condensing the amino-alpha, omega-dicarboxylic acid, with or without catalysts at 110-180° C. and removing the water of condensation under vacuum, for example, 1 mm Hg-100 mm Hg, to produce 1000-5000 molecular weight polymer or prepolymer.

Catalysts can be used for polymers of amino-alpha, omega-dicarboxylic acid formation. For example, catalysts that can be used include, tin oxide (SnO), Sn(II) octoate, Li carbonate, Zn diacetate dehydrate, Ti(tetraisopropoxide), potassium carbonate, tin powder, combinations thereof and mixtures of these. Catalysts can be used in combination and/or sequentially.

The cyclic monomer can be ring open polymerized (ROP) by solution, bulk, melt and suspension polymerization and is catalyzed by cationic, anionic, coordination or free radical polymerization. Some catalysts used, for example, include protonic acids, HBr, HCl, triflic acid, Lewis acids, ZnCl₂, AlCl₃, anions, potassium benzoate, potassium phenoxide, potassium t-butoxide, and zinc stearate, metals, Tin, zinc, aluminum, antimony, bismuth, lanthanide and other heavy metals, Tin (II) oxide and tin (II) octoate (e.g., 2-ethylhexanoate), tetraphenyl tin, tin (II) and (IV) halogenides, tin (II) acetylacetonoate, distannoxanes (e.g., hexabutyldistannoxane, R₃SnOSnR₃ where R groups are alkyl or aryl groups), Al(OiPr)₃, other functionalized aluminum alkoxides (e.g., aluminum ethoxide, aluminum methoxide), ethyl zinc, lead (II) oxide, antimony octoate, bismuth octoate, rare earth catalysts, yttrium tris(methyl lactate), yttrium tris(2-N—N-dimethylamino ethoxide), samarium tris(2-N—N-dimethylamino ethoxide), yttrium tris(trimethylsilylmethyl), lanthanum tris(2,2,6,6-tetramethylheptanedionate), lanthanum tris(acetylacetonoate), yttrium octoate, yttrium tris(acetylacetonate), yttrium tris(2,2,6,6-tetramethylheptanedionate), combinations of these (e.g., ethyl zinc/aluminum isopropoxide) and mixtures of these

In addition to homopolymer, copolymerization with other cyclic monomers and non-cyclic monomers such as glycolide, caprolactone, valerolactone, dioxypenone, trimethyl carbonate, 1,4-benzodioxepin-2,5-(3H)-dione glycosalicylide, 1,4-benzodioxepin-2,5-(3H,3-methyl)-dione lactosalicylide, dibenzo-1,5 dioxacin-6-12-dione disalicylide, morpholine-2,5-dione, 1,4-dioxane-2,5-dione glycolide, oxepane-2-one ε-caprolactone, 1,3-dioxane-2-one trimethylene carbonate, 2,2-dimethyltrimethylene carbonate, 1,5-dioxepane-2-one, 1,4-dioxane-2-one p-dioxanone, gamma-butyrolactone, beta-butyrolactone-Me-delta-valerolactone, 1,4-dioxane-2,3-dione ethylene oxalate, 3-[benzyloxycarbonyl methyl]-1,4-dioxane-2,5-dione, ethylene oxide, propylene oxide, 5,5′(oxepane-2-one), 2,4,7,9-tetraoxa-spiro[5,5]undecane-3,8-dione Spiro-bid-dimethylene carbonate can produce co-polymers. Copolymers can also be produced by adding monomers such as the multifunctional hydroxyl, carboxylic compounds or the heterofunctional compounds that can be used as coupling agents for low molecular weight polymers of amino-alpha, omega-dicarboxylic acid.

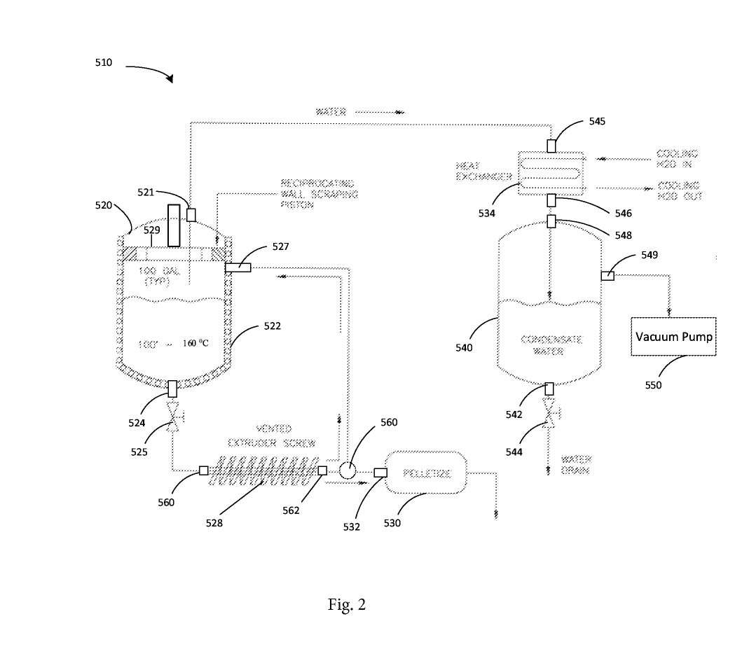

FIG. 2 shows a schematic view of a reaction system for polymerizing amino-alpha, omega-dicarboxylic acid. The reaction system (510) includes a stainless steel jacked reaction tank (520), a vented screw extruder (528), a pelletizer (530), a heat exchanger (534) and a condensation tank (540). An outlet (521) of the reaction tank is connected to a tube (e.g., stainless steel) which is connected to an inlet (545) to a heat exchanger. An outlet (546) to the heat exchanger is connected to another tube (e.g., stainless steel) and is connected to an inlet (548) to the condensation tank (540). The tubes and connections from the reaction tank and condensation tank provide a fluid pathway (e.g., water vapor/air) between the two tanks. A vacuum can be applied to the fluid pathway between the tanks (520) and (540) by utilizing a vacuum pump (550) that is connected to port (549).

The reaction tank (520) includes an outlet (524) that can be connected to a tube (e.g., stainless steel) that is connected to an inlet to a screw extruder (560). An outlet to the extruder (562) is connected to a tube which is connected optionally through a valve (560) to the reaction tank (520) through inlet (527). Optionally the outlet to the extruder (562) is connected through valve (560) to the pelletizer (530) through inlet (532). Tubes and connections from the reaction tank and extruder provide a circular fluid pathway (e.g., reactants and products) between the reaction tank and extruder when the valve (560) is set in recirculating position. The tubes and connections from the reaction tank to the pelletizer provide a fluid pathway between the reaction tank and pelletizer when the valve (560) is set in pelletizing position.

When in operation, the tank can be charged with amino-alpha, omega-dicarboxylic acid. The amino-alpha, omega-dicarboxylic acid is heated in the tank utilizing the stainless steel heating jacket (522). In addition, a vacuum is applied to the condensation tank (540) and therefore to the reaction tank (520) through the stainless steel tubing and connections using the vacuum pump (550). The heating of the amino-alpha, omega-dicarboxylic acid accelerates the condensation reactions (e.g., esterification reactions) to form oligomers of amino-alpha, omega-dicarboxylic acid while the applied vacuum helps volatilize the water that is produced. Water vapor travels out of the reactants and out of the reaction tank (520) and towards the heat exchanger (534) as indicated by the arrow. The heat exchanger cools the water vapor and the condensed water drops into the condensation tank (540) through the tubes and connections previously described. Multiple heat exchangers can be utilized. Since the amino-dicarboxylic acids can be corrosive the reactor equipment and other associated equipment may be clad or coated with corrosive resistant metals such as tantalum, alloys such as HASTELLOY™, a trademarked alloy from Haynes International, and the like. It can also be coated with inert high temperature polymeric coatings such as TEFLON™ from DuPont, Wilmington De. Also, water undoubtedly hydrates the acid and the acid end of the polymer. When those waters of hydration are removed the acidity can be much higher, since it is not leveled by the waters of hydration.

In addition, during operation, extruder (528) can be engaged and operated to draw the reactants (e.g., amino-alpha, omega-dicarboxylic acid, oligomers and polymers) out of the tank. When the valve (560) is set in recirculating position the reactants are circulated back to the reaction tank in the direction shown by the arrows. In addition to the extruder, the flow can be controlled by valve (525), for example, the valve can be set to closed for no flow, open for maximal flow or an intermediate position for lower or high flow rates (e.g., between about 0 and 100% open, e.g., about 0%, 10%, 20%, 30%, 40%, 50%, 60%, 70%, 80%, 90% or about 100% open).

The reaction can be continued with reactants following a circular pathway (e.g., with valve in recirculating position) until a desired polymerization is achieved. This circulating pathway provides mixing and shearing that can help the polymerization (e.g., increase molecular weight, control polydispersity, improve the kinetics of the polymerization, improve temperature distribution and diffusion of reacting species). The products (e.g. polymer) can then be directed to the pelletizer by setting valve (560) to the pelletizing position. The pelletizer then can produce pellets which can be collected. Pellets can be of various shapes and sizes. For example, spherical or approximately spherical, hollow tube shaped, filled tube shape with, for example, approximate volumes, between about 1 mm³ to about 1 cm³. The pelletizer can also be replaced with other equipment, for example, extruders, mixers, reactors, and filament makers.

The extruder (528) can be a vented screw extruder so that water or other volatile compounds can be removed from further processing. The extruder can be a single screw extruder or a multiple screw extruder. For example, the extruder can be a double screw extruder with co-rotating or counter rotating screws. The screw extruder can also be a hollow flight extruder and can be heated or cooled. The screw extruder can be fitted with ports to its interior. The ports can be utilized, for example, for the addition of additives, addition of co-monomers, addition of cross-linking agents, addition of catalysts, irradiation treatments and addition of solvents. The ports can also be utilized for sampling (e.g., to test the progress of the reaction or troubleshoot). In addition to sampling, the torque applied to the extruder can be used to monitor the progress of the polymerization (e.g., as the viscosity increases). An inline mixer such as a static mixer can also be disposed in the pathway of the circulating reactants, for example, before or after the screw extruder, providing a tortuous path for the reactants which can improve the mixing supplied to the reactants.

The position of the return port (527) allows the reactants to flow down the side of the tank, increasing the surface area of the reactants facilitating the removal of water. The return port can include multiple (e.g. a plurality of ports) disposed at various positions in the tanks. For example, the plurality of return ports can be placed circumferentially around the tank.

The tank can include a reciprocating scraper (529) which can help push the formed polymer/oligomers down the reaction tank, for example, during or after completion of the reaction. Once the reciprocating scraper moves down, the scraper can then be moved back up, for example, to a resting position. The scraper can be moved up and down the tank by engaging with and axel (640) that is attached to the hub (650). In another possible embodiment, the hub can be tapped for mechanical coupling to a screw, for example, wherein the axel is a screw-axel that extends to the bottom of the tank. The screw-axel can then turn to drive the scraper down or up.

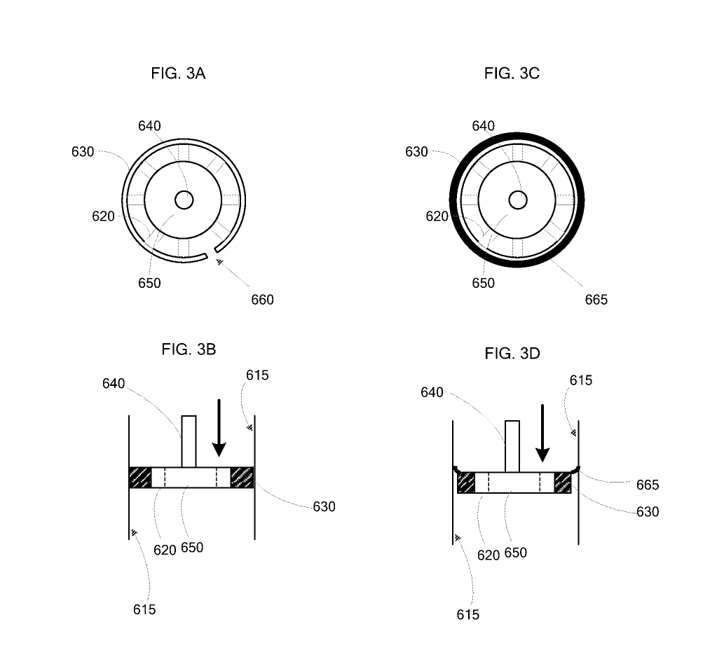

A top view of one embodiment of a reciprocating scraper is shown in FIG. 3A while a front cut out view is shown in FIG. 3B. The reciprocating scraper includes pistons (620) attached to a hub (650) and scraping ends (630). The scraping end is in the form of a compression ring with a gap (660). The pistons apply pressure against the inside surfaces of the tank (615) through the scraping ends (630) while the scraper can be moved down the tank as shown by the arrow in FIG. 3B. The gap (660) allows the expansion and contraction of the scraper. The scraper can be made of any flexible material, for example, steel such as stainless steel. The gap is preferably as small as possible (e.g., less than about 1″, less than about 0.1″, less than about 0.01″ or even less than about 0.001″).

Another embodiment of a reciprocating scraper is shown in FIG. 3C and FIG. 3D. In this second embodiment the scraping ends include a lip-seal. The lip seal can be made of a flexible material, for example, rubber. The movement of the lip-seal as the scraper moves up and down acts as a squeegee against the inside of the reaction tank.

The tank (520) can be 100 gal in size, although larger and smaller sizes can be utilized (e.g., between about 20 to 10,000 gal, e.g., at least 50 gal, at least 200 gal, at least 500 gal, at least 1000 gal). The tank, for example, can be shaped with a conical bottom or rounded bottom.

In addition to the inlets and outlets discussed, the tank can also include other openings, for example, to allow the addition of reagents or for access to the interior of the tank for repairs.

During the reaction the temperature in the tank can be controlled from between about 100 and 220° C. The polymerization can preferably started at about 100° C. and the temperature increased to about 200° C. over several hours (e.g., between 1 and 48 hours, 1 and 24 hours, 1 and 16 hours, 1 and 8 hours). A vacuum can be applied between about 0.1 and 2 mmHg). For example, at the beginning of the reaction about 0.1 mmHg and at the end of the reaction about 2 mmHg.

Water from the condenser tank (540) can be drained trough an opening (542) utilizing control valve (544).

The heat exchanger can be a fluid cooled heat exchanger. For example, cooled with water, air or oil. Several heat exchangers can be used, for example, as needed to condense as much of the water as possible. For example, a second heat exchanger can be located between the vacuum pump and the condensation tank (540).

The equipment and reactions described herein (e.g., FIG. 2 can also be used for polymerization of other monomers. In addition, the equipment can be utilized after or during the polymerizations for blending of polymers.

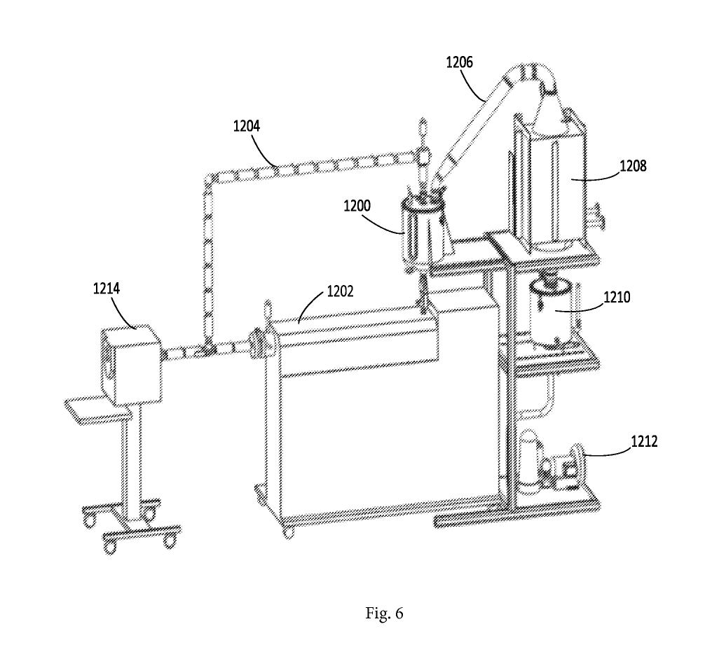

FIG. 6 is a schematic of a polymerization system for polymerizing or co-polymerizing e.g., amino-alpha, omega-dicarboxylic acid. The thin film evaporator or thin film polymerization/devolatilization device 1200, and (optional) extruder 1202 for product isolation or recycle back to the thin film evaporator or thin film polymerization/devolatilization device, a heated recycle loop 1204, a heated condenser 1206, cooled condenser 1208 for condensing water and other volatile components, a collection vessel 1210 a fluid transfer unit 1212 (e.g., including a pump) to remove condensed water and volatile components and a product isolation device 1214. The effluent from 1212 can optionally be taken to a another unit operation to recover the useful volatile components for recycle back to polymerization steps, for example, the first step discussed above. The thin film evaporator or thin film polymerization/devolatilization device is preferably utilized in the third step describe above. The fluid transfer unit is shown as a pump.

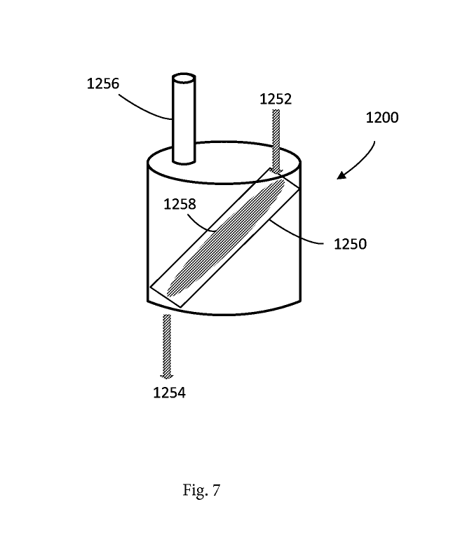

FIG. 7 is a cutaway of the thin film polymerization/devolatilization device. The angled rectangular piece 1250 is the optionally heated surface where the molten polymer flows. The incoming molten polymer stream 1252 flows onto the surface and is shown as an ellipse 1258 of flowing polymer flowing to the exit of the device at 1254. The volatiles are removed through pipe 1256.

The internals of the thin film evaporator or thin film polymerization/devolatilization device can be in different configurations, but can be configured to assure that the polymer fluid flows in a thin film through the device. This is to facilitate volatilization of the water that is in the polymer fluid or is formed by a condensation reaction. For instance, the surface may be slanted at an angle relative to the straight sides of the device. The surface may be separately heated such that the surface is 0 to 40° C. hotter than the polymer fluid. With this heated surface it can be heated to up to 300° C., as much as 40° C. higher than the overall temperature of the device.

The thickness of the polymer fluid flowing along the thin film part of the device is less than 1 cm, optionally less than 0.5 cm or alternately less than 0.25 cm.

The thin film evaporator and thin film polymerization/devolatilization device are similar in function. Other similar devices similar in function should be considered to have the same function as these. Descriptively, these include wiped film evaporators (e.g., as previously described), short path evaporator, a shell and tube heat exchanger and the like. For each of these evaporator configurations a distributor may be used to assure distribution of the thin film. The limitation that they must be able to operate at the conditions described above.

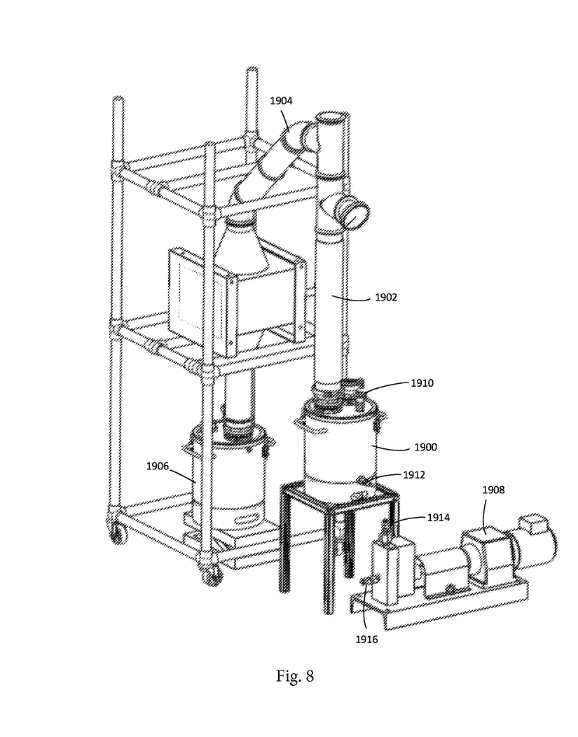

FIG. 8 is a schematic of a pilot-scale polymerization system to polymerize amino-alpha, omega-dicarboxylic acid. The thin film evaporator or thin film polymerization/devolatilization device 1900, a heated riser 1902, a cooled condenser 1904 for condensing water and other volatile components, a collection vessel 1906 a fluid transfer unit 1908 to recycle the polymer fluid shown as a pump. The connecting tubing is not shown for clarity. The output of the pump 1916 is connected to inlet 1910, the device output 1912 is connected to the inlet of the pump 1914. The product isolation section is not shown. Internal in the thin film polymerization/devolatilization device is a slanted surface. The polymer fluid is flowed to the inlet with the configured such that the polymer fluid flows onto the slanted surface. This slanted surface may be separately heated as described above.

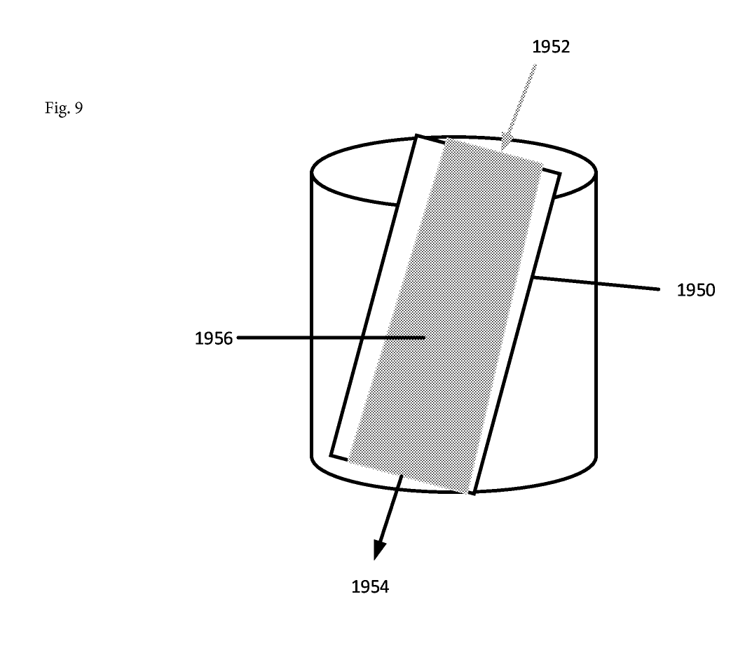

FIG. 9 is a cutaway of the thin film polymerization/devolatilization device. The angled rectangular piece 1950 is the optionally heated surface where the molten polymer flows. The incoming molten polymer stream 1952 flows onto the surface and is shown as a trapezoid 1956 of flowing polymer flowing to the exit of the device at 1954.

The thin film polymerization/devolatilization device is configured such that fluid polymer is conveyed to the device such that the film of the fluid polymer is less than 1 cm thick and provides a means for volatilizing the water formed in the reaction and other volatile components. The temperature of the thin film evaporator and polymerization/devolatilization device are from 100 to 240° C. and the pressure of the device is from 0.000014 to 50 kPa. A full vacuum may be used in the evaporator device. Pressures can be e.g., less than 0.01 torr, alternatively less than 0.001 torr and optionally less than 0.0001 torr.

### Stereochemistry Polymers of Amino-Alpha, Omega-Dicrboxylic Acid

Mechanical and thermal properties of polymers of amino-alpha, omega-dicarboxylic acid are influenced by the molecular weight and stereochemical composition of the backbone. The stereochemical composition of the backbone can be controlled by the choice and ratios of monomers; D-amino-alpha, omega-dicarboxylic acid, L-polymers of amino-alpha, omega-dicarboxylic acid and whether the alpha or the omega carboxylic acid is part of the backbone.

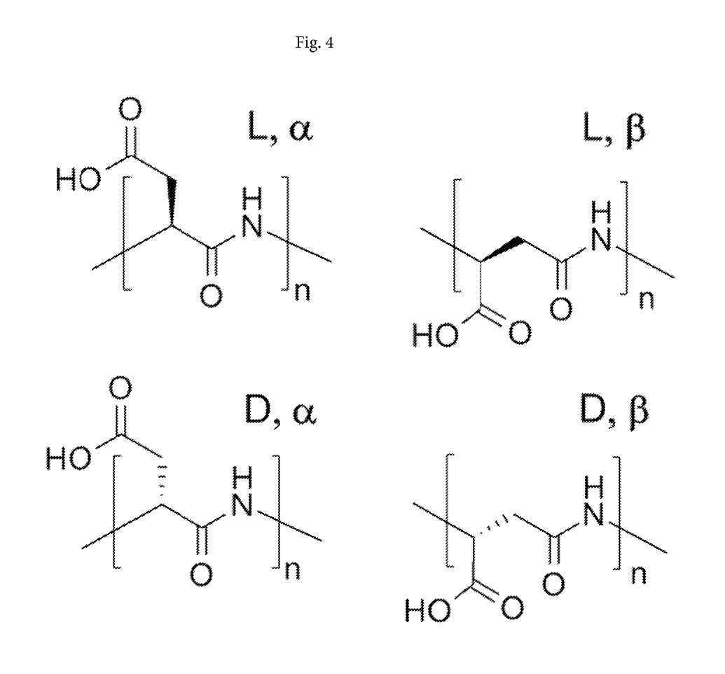

The molecular weight of the polymers can be controlled, for example, as discussed above. FIG. 4 shows the polymer products of the polyamide as shown for aspartic acid. Other polyamides will have similar configurations with amide linkages being to the alpha carboxylate and omega carboxylate. For instance, for aspartic acid the omega carboxylate is at the beta position and for glutamic acid the omega carboxylate is at the gamma position.

The thermal treatment of samples, for example, rates of melting, recrystallization, and annealing history, can in part determine the amount of crystallization. Therefore, comparisons of the thermal, chemical and mechanical properties of polymers of amino-alpha, omega-dicarboxylic acid should generally be most meaningful for polymers with a similar thermal history.

### Copolymers, Crosslinking and Grafting of Polymers of Amino-Alpha, Omega Dicarboxylic Acids

Variation of polymers of amino-alpha, omega-dicarboxylic acid by the formation of copolymers as discussed above also has a very large influence on the properties, for example, by disrupting and decreasing the crystallinity and modulating the glass transition temperatures. For example, polymers with increased flexibility, improved hydrophilicity, better degradability, better biocompatibility, better tensile strengths, and improved elongations properties can be produced.

Some additional useful monomers that have been copolymerized with amino-alpha, omega-dicarboxylic acid include 1,4-benzodioxepin-2,3(H)-dione glycosalicylide; 1.3-benzodioxepin-2,5-(3H,3-methyl)-dione lactosalicylide; dibenzo-1,5-dioxacin-6,12-dione disalicylide; morpholine-2,5-dione, 1,4-dioxane-2,5-dione, glycolide; oxepane-2-one trimethylene carbonate; 2,2-dimethyltrimethylene carbonate; 1,5-dioxepane-2-one; 1,4-dioxane-2-one p-dioxanone; gamma-butyrolactone; beta-butyrolactone; beta-methyl-delta-valerolactone; beta-methyl-gamma-valerolactone; 1,4-dioxane-2,3-dione ethylene oxalate; 3[(benzyloxyacarbonyl)methyl]-1,4-dioxane-2,5-dione; ethylene oxide; propylene oxide, 5,5′-(oxepane-2-one) and 2,4,7,9-tetraoxa-spiro[5,5]undecane-3,8-dione Spiro-bis-dimethylene carbonate.

The amino-alpha, omega-dicarboxylic acid polymers and co-polymers can be modified by cross linking Cross linking can affect the thermal and rheological properties without necessarily deteriorating the mechanical properties. For example, 0.2 mol % 5,5′-bis(oxepane-2-one)(bis-ε-caprolactone)) and 0.1-0.2 mol % spiro-bis-dimethylene carbonate cross linking Free radical hydrogen abstraction reactions and subsequent polymer radical recombination is an effective way of inducing crosslinks into a polymer. Radicals can be generated, for example, by high energy electron beam and other irradiation (e.g., between about 0.01 Mrad and 15 Mrad, e.g. between about 0.01-5 Mrad, between about 0.1-5 Mrad, between about 1-5 Mrad). For example, irradiation methods and equipment are described in detail below. Crosslinking can also be achieved with the inclusion of tri-substituted monomers in modest amounts. The amounts of tri-substituted monomers can be less than 5 wt. % based on the aspartic acid, alternately less than 3 wt. %.

Alternatively or in addition, peroxides, such as organic peroxides are effective radical producing and cross linking agents. For example, peroxides that can be used include hydrogen peroxide, dicumyl peroxide; a,a′-bis(tert-butylperoxy)-diisopropylbenzene; benzoyl peroxide; 2,5-dimethyl-2,5-di(tert-butylperoxy)hexane; tert-butylperoxy 2-ethylhexyl carbonate; tert-Amyl peroxy-2-ethylhexanoate; 1,1-di(tert-amylperoxy)cyclohexane; tert-amyl peroxyneodecanoate; tert-amyl peroxybenzoate; tert-amylperoxy 2-ethylhexyl carbonate; tert-amyl peroxyacetate; 2,5-dimethyl-2,5-di(2-ethylhexanoylperoxy)hexane; tert-butyl peroxy-2-ethylhexanoate; 1,1-di(tert-butylperoxy)cyclohexane; tert-butyl peroxyneodecanoate; tert-butyl peroxyneoheptanoate; tert-Butyl peroxydiethylacetate; 1,1-di(tert-butylperoxy)-3,3,5-trimethylcyclohexane; 3,6,9-triethyl-3,6,9-trimethyl-1,4,7-triperoxonane; di(3,5,5-trimethylhexanoyl) peroxide; tert-butyl peroxyisobutyrate; tert-butyl peroxy-3,5,5-trimethylhexanoate; di-tert-butyl peroxide; tert-butylperoxy isopropyl carbonate; tert-butyl peroxybenzoate; 2,2-di(tert-butylperoxy)butane; di(2-ethylhexyl) peroxydicarbonate; di(2-ethylhexyl) peroxydicarbonate; tert-butyl peroxyacetate; tert-butyl cumyl peroxide; tert-amylhydroperoxide; 1,1,3,3-tetramethylbutyl hydroperoxide, and mixtures of these. The effective amounts can vary, for example, depending on the peroxide, cross-linking reaction conditions and the desired properties (e.g., amount of cross linking) For example, cross-linking agents can be added from between about 0.01-10 wt. % (e.g., about 0.1-10 wt. %, about 0.01-5 wt. %, about 0.1-1 wt. %, about 1-8 wt. %, about 4-6 wt. %). For example, peroxides such as 5.25 wt. % dicumyl peroxide and 0.1% benzoyl peroxide are effective radical producing and cross linking agents for amino-alpha, omega-dicarboxylic acid and amino-alpha, omega-dicarboxylic acid derivatives. The peroxide cross-linking agents can be added to polymers as solids, liquids or solutions, for example, in water or organic solvents such as mineral spirits. In addition radical stabilizers can be utilized.

Cross linking can also be effectively accomplished by the incorporation of unsaturation in the polymer chain either by: initiation with unsaturated alcohols such as hydroxyethyl methacrylate or 2-butene-1,4-diol; the post reaction with unsaturated anhydrides such as maleic anhydride; or copolymerization with unsaturated epoxides such as glycidyl methacrylate.

In addition to cross linking, grafting of functional groups and polymers to amino-alpha, omega-dicarboxylic acid polymer or co-polymer is an effective method of modifying the polymer properties. For example, radicals can be formed as described above and a monomer, functionalizing polymer or small molecule. For example, irradiation or treatment with a peroxide and then quenching with a functional group containing an unsaturated bond can effectively functionalize the amino-alpha, omega-dicarboxylic acid backbone.

### Blending Polymers of Amino-Alpha, Omega-Dicarboxylic Acid

Amino-alpha, omega-dicarboxylic acid can be blended with other polymers as miscible or immiscible compositions. For immiscible blends the composition can be one with the minor component (e.g., below about 30 wt. %) as small (e.g., micron or sub-micron) domains in the major component. When one component is about 30 to 70 wt. % the blend can form a co-continuous morphology (e.g., lamellar, hexagon phases or amorphous continuous phases). For instance, addition of polyaspartic acid to poly lactic acid can accelerate degradation and improvement of thermal stability.

Blending can be accomplished by melt mixing above the glass transition temperature of the amorphous polymer components. Screw extruders (e.g., single screw extruders, co-rotating twin screw extruders, counter rotating twin screw extruders) can be useful for this.

Polyethylene oxide (PEO) and polypropylene oxide (PPO) can be blended with amino-alpha, omega-dicarboxylic acid. Lower molecular weight glycols (300-1000 Mw) are miscible with amino-alpha, omega-dicarboxylic acid while PPO becomes immiscible at higher molecular weight. These polymers, especially PEO, can be used to increase the water transmission and bio-degradation rate of polymers of amino-alpha, omega-dicarboxylic acid. They can also be used as polymeric plasticizers to lower the modulus and increase flexibility of polymers of amino-alpha, omega-dicarboxylic acid.

Blends of polymers of amino-alpha, omega-dicarboxylic acid and polyolefins (polypropylene and polyethylene) can result in incompatible systems with poor physical properties due to the poor interfacial compatibility and high interfacial energy. However, the interfacial energy can be lowered, for example, by the addition of third component compatibilizers, such as glycidyl methacrylate grafted polyethylene. Polystyrene and high impact polystyrene resins are also non-polar and blends with polymers of amino-alpha, omega-dicarboxylic acid are generally not very compatible.

Polymers of amino-alpha, omega-dicarboxylic acid and acetals can be blended making compositions with useful properties.

Polymers of amino-alpha, omega-dicarboxylic acid may be miscible with polymethyl methacrylate and many other acrylates and copolymers of (meth)acrylates. Drawn films of PMMA/polymers of amino-alpha, omega-dicarboxylic acid blends can be transparent and have high elongation.

Polycarbonate can be combined with polymers of amino-alpha, omega-dicarboxylic acid. The compositions may have high heat resistance, flam resistance and toughness and have applications, for example, in consumer electronics such as laptops.

Acrylonitrile butadiene styrene (ABS) can be blended with polymers of amino-alpha, omega-dicarboxylic acid although the polymers may not miscible.

Poly(propylene carbonate) can be blended with polymers of amino-alpha, omega-dicarboxylic acid providing a biodegradable composite since both polymers are biodegradable.

PASA can also be blended with poly(butylene succinate). Blends can impart thermal stability and impact strength to the polymers of amino-alpha, omega-dicarboxylic acid.

PEG, poly propylene glycol, poly (vinyl acetate), anhydrides (e.g., maleic anhydride) and fatty acid esters have been added as plasticizers and/or compatibilizers.

Blending can also be accomplished with the application of irradiation, including irradiation and quenching. For example, irradiation or irradiation and quenching as described herein applied to biomass can be applied to the irradiation of polymers of amino-alpha, omega-dicarboxylic acid and polymers of amino-alpha, omega-dicarboxylic acid copolymers for any purpose, for example, before, after and/or during blending. This treatment can aid in the processing, for example, making the polymers more compatible and/or making/breaking bonds within the polymer and/or blended additive (e.g., polymer, plasticizer). For example, between about 0.1 Mrad and 150 Mrad followed by quenching of the radicals by the addition of fluids or gases (e.g., oxygen, nitrous oxide, ammonia, liquids), using pressure, heat, and/or the addition of radical scavengers. Quenching of biomass that has been irradiated is described in U.S. Pat. No. 8,083,906 to Medoff, the entire disclosure of which is incorporate herein by reference, and the equipment and processes describe therein can be applied to polymers of amino-alpha, omega-dicarboxylic acid and polymers of amino-alpha, omega-dicarboxylic acid derivatives. Irradiation and extruding or conveying of the polymers of amino-alpha, omega-dicarboxylic acid or polymers of amino-alpha, omega-dicarboxylic acid copolymers can also be utilized, for example, as described for the treatment of biomass in U.S. application Ser. No. 13/009,151 filed on May 2, 2011 the entire disclosure of which is incorporated herein by reference.

### Composites of Polymers of Amino-Alpha, Omega-Dicarboxylic Acids

Polymers of amino-alpha, omega-dicarboxylic acid polymers, co-polymers and blends can be combined with synthetic and/or natural materials. For example, polymers of amino-alpha, omega-dicarboxylic acid and any polymers of amino-alpha, omega-dicarboxylic acid derivative (e.g., polymers of amino-alpha, omega-dicarboxylic acid copolymers, polymers of amino-alpha, omega-dicarboxylic acid blends, grated polymers of amino-alpha, omega-dicarboxylic acid, cross-linked polymers of amino-alpha, omega-dicarboxylic acid) can be combined with synthetic and natural fibers. For example, protein, starch, cellulose, plant fibers (e.g., abaca, leaf, skin, bark, kenaf fibers), inorganic fillers, flax, talc, glass, mica, saponite and carbon fibers. This can provide a material with, for example, improved mechanical properties (e.g., toughness, harness, strength) and improved barrier properties (e.g., lower permeability to water and/or gasses).

Nano composites can also be made by dispersing inorganic or organic nanoparticles into either a thermoplastic or thermoset polymer. Nanoparticles can be spherical, polyhedral, two dimensional nanofibers or disc-like nanoparticles. For example, colloidal or microcrystalline silica, alumina or metal oxides (e.g., TiO₂); carbon nanotubes; clay platelets.

Composites can be prepared similarly to polymer blends, for example, utilizing screw extrusion and/or injection molding. Irradiation as described herein can also be applied to the composites, during, after or before their formation. For example irradiation of the polymer and combination with the synthetic and/or natural materials, or irradiation of the synthetic and/or natural materials and combination with the polymer, or irradiation of both the polymer and synthetic and/or natural material and then combining, or irradiating the composite after it has been combined, with or without further processing. Polyaspartic acid can also be used with in silk fibroin films to facilitate hydroxyapatite deposition.

Pla with Plasticizers and Elastomers

In addition to the blends previously discussed, polymers of amino-alpha, omega-dicarboxylic acid and polymers of amino-alpha, omega-dicarboxylic acid derivatives can be combined with plasticizers.

Polymers of amino-alpha, omega-dicarboxylic acid can be blended with monomeric and oligomeric plasticizers. Monomeric plasticizers, such as tributyl citrate, TbC, and diethyl bishydroxymethyl malonate, DBM, may decrease the T_{g} of PASA. Increasing the molecular weight of the plasticizers by synthesizing oligoesters and oligoesteramides can result in blends with T_{g} depressions slightly smaller than with the monomeric plasticizers. The compatibility with polymers of amino-alpha, omega-dicarboxylic acid can be dependent on the molecular weight of the oligomers and on the presence of polar groups (e.g., amide groups, hydroxyl groups, ketones, esters) that can interact with the polymers of amino-alpha, omega-dicarboxylic acid chains. The materials can retain a high flexibility and morphological stability over long periods of time, for example, when formed into films.

Some examples of elastomers that can be combined with PLA include: Elastomer NPEL001, Polyurethane elastomers (5-10%), Functionalized polyolefin elastomers, Blendex® (e.g., 415, 360, 338), PARALOID™ KM 334, BTA 753, EXL 3691A, 2314, Ecoflex® Supersoft Silicone Bionolle® 3001, Pelleethane® 2102-75A, Kraton® FG 1901X, Hytrel® 3078, and mixtures of these. Mixtures with any other elastomer, for example, as described herein can also be used.

Some examples of plasticizers that can be combined with polymers of amino-alpha, omega-dicarboxylic acid include: Triacetine, Glycerol triacetate, Tributyl citrate, Polyethylene glycol, GRINDSTED® SOFT-N-SAFE (acetic acid ester of monoglycerides) made from fully hydrogenated castor oil and combinations of these. Mixtures with any other plasticizers, for example, as described herein can also be used.

The main characteristic of elastomer materials is the high elongation and flexibility or elasticity of these materials, against its breaking or cracking.

Depending on the distribution and degree of the chemical bonds of the polymers, elastomeric materials can have properties or characteristics similar to thermosets or thermoplastics, so elastomeric materials can be classified into: thermoset elastomers (e.g., do not melt when heated) and thermoplastic elastomers (e.g., melt when heated). Some properties of elastomer materials: cannot melt, before melting they pass into a gaseous state; swell in the presence of certain solvents; Are generally insoluble; are flexible and elastic; lower creep resistance than the thermoplastic materials.

Examples and applications of elastomer materials are: polyurethanes are used in the textile industry for the manufacture of elastic clothing such as Lycra®, also used as foam, and for wheels; polybutadiene-elastomer material used on the wheels or tires of vehicles, given the extraordinary wear resistance; Neoprene-Material used primarily in the manufacture of wetsuits is also used as wire insulation, industrial belts; silicone-material used in a wide range of materials and areas due their excellent thermal and chemical resistance, silicones are used in the manufacture of pacifiers, medical prostheses, lubricants.

Some examples of elastomers adhesives are: polyurethane adhesive 2 components; polyurethane adhesive by curing 1 component moisture; adhesives based on silicones; adhesives based on modified silane.

### Flavors, Fragrances and Colors

Any of the products and/or intermediates described herein, for example, amino-alpha, omega-dicarboxylic acids, aspartic acid, glutamic acid, polymers of amino-alpha, omega-dicarboxylic acid, polymers of amino-alpha, omega-dicarboxylic acid derivatives (e.g., polymers of amino-alpha, omega-dicarboxylic acid copolymers, polymers of amino-alpha, omega-dicarboxylic acid composites, cross-linked polymers of amino-alpha, omega-dicarboxylic acid, grafted polymers of amino-alpha, omega-dicarboxylic acid, polymers of amino-alpha, omega-dicarboxylic acid blends or any other polymers of amino-alpha, omega-dicarboxylic acid containing material prepared as described herein) can also be combined with flavors, fragrances colors and/or mixtures of these. For example, any one or more of (optionally along with flavors, fragrances and/or colors) sugars, organic acids, fuels, polyols, such as sugar alcohols, biomass, fibers and composites amino-alpha, omega-dicarboxylic acids, aspartic acid, glutamic acid, polymers of amino-alpha, omega-dicarboxylic acid, polymers of amino-alpha, omega-dicarboxylic acid derivatives can be combined with (e.g., formulated, mixed or reacted) or used to make other products. For example, one or more such product can be used to make soaps, detergents, candies, drinks (e.g., cola, wine, beer, liquors such as gin or vodka, sports drinks, coffees, teas), pharmaceuticals, adhesives, sheets (e.g., woven, none woven, filters, tissues) and/or composites (e.g., boards). For example, one or more such product can be combined with herbs, flowers, petals, spices, vitamins, potpourri, or candles. For example, the formulated, mixed or reacted combinations can have flavors/fragrances of grapefruit, orange, apple, raspberry, banana, lettuce, celery, cinnamon, vanilla, peppermint, mint, onion, garlic, pepper, saffron, ginger, milk, wine, beer, tea, lean beef, fish, clams, olive oil, coconut fat, pork fat, butter fat, beef bouillon, legume, potatoes, marmalade, ham, coffee and cheeses.

Flavors, fragrances and colors can be added in any amount, such as between about 0.01 wt. % to about 30 wt. %, e.g., between about 0.05 to about 10, between about 0.1 to about 5, or between about 0.25 wt. % to about 2.5 wt. %. These can be formulated, mixed and or reacted (e.g., with any one of more product or intermediate described herein) by any means and in any order or sequence (e.g., agitated, mixed, emulsified, gelled, infused, heated, sonicated, and/or suspended). Fillers, binders, emulsifier, antioxidants can also be utilized, for example protein gels, starches and silica.

The flavors, fragrances and colors can be natural and/or synthetic materials. These materials can be one or more of a compound, a composition or mixtures of these (e.g., a formulated or natural composition of several compounds). Optionally the flavors, fragrances, antioxidants and colors can be derived biologically, for example, from a fermentation process (e.g., fermentation of saccharified materials as described herein). Alternatively or additionally these flavors, fragrances and colors can be harvested from a whole organism (e.g., plant, fungus, animal, bacteria or yeast) or a part of an organism. The organism can be collected and or extracted to provide color, flavors, fragrances and/or antioxidant by any means including utilizing the methods, systems and equipment described herein, hot water extraction, chemical extraction (e.g., solvent or reactive extraction including acids and bases), mechanical extraction (e.g., pressing, comminuting, filtering), utilizing an enzyme, utilizing a bacteria such as to break down a starting material, and combinations of these methods. The compounds can be derived by a chemical reaction, for example, the combination of a sugar (e.g., as produced as described herein) with an amino acid (Maillard reaction). The flavor, fragrance, antioxidant and/or color can be an intermediate and or product produced by the methods, equipment or systems described herein, for example and ester and a lignin derived product.

Some examples of flavor, fragrances or colors are polyphenols. Polyphenols are pigments responsible for the red, purple and blue colors of many fruits, vegetables, cereal grains, and flowers. Polyphenols also can have antioxidant properties and often have a bitter taste. The antioxidant properties make these important preservatives. On class of polyphenols are the Flavonoids, such as Anthrocyanies, flavonols, flavan-3-ols, flavones, flavanones and flavanononols. Other phenolic compounds that can be used include phenolic acids and their esters, such as chlorogenic acid and polymeric tannins.

Inorganic compounds, minerals or organic compounds can be used, for example titanium dioxide, cadmium yellow (E.g., CdS), cadmium orange (e.g., CdS with some Se), alizarin crimson (e.g., synthetic or non-synthetic rose madder), ultramarine (e.g., synthetic ultramarine, natural ultramarine, synthetic ultramarine violet), cobalt blue, cobalt yellow, cobalt green, viridian (e.g., hydrated chromium(III)oxide), chalcophyllite, conichalcite, cornubite, cornwallite and liroconite.

Some flavors and fragrances that can be utilized include ACALEA TBHQ, ACET C-6, ALLYL AMYL GLYCOLATE, ALPHA TERPINEOL, AMBRETTOLIDE, AMBRINOL 95, ANDRANE, APHERMATE, APPLELIDE, BACDANOL®, BERGAMAL, BETA IONONE EPDXIDE, BETA NAPHTHYL ISO-BUTYL ETHER, BICYCLONONALACTONE, BORNAFIX®, CANTHOXAL, CASHMERAN®, CASHMERAN® VELVET, CASSIFFIX®, CEDRAFIX, CEDRAMBER®, CEDRYL ACETATE, CELESTOLIDE, CINNAMALVA, CITRAL DIMETHYL ACETATE, CITROLATE™, CITRONELLOL 700, CITRONELLOL 950, CITRONELLOL COEUR, CITRONELLYL ACETATE, CITRONELLYL ACETATE PURE, CITRONELLYL FORMATE, CLARYCET, CLONAL, CONIFERAN, CONIFERAN PURE, CORTEX ALDEHYDE 50% PEOMOSA, CYCLABUTE, CYCLACET®, CYCLAPROP®, CYCLEMAX™, CYCLOHEXYL ETHYL ACETATE, DAMASCOL, DELTA DAMASCONE, DIHYDRO CYCLACET, DIHYDRO MYRCENOL, DIHYDRO TERPINEOL, DIHYDRO TERPINYL ACETATE, DIMETHYL CYCLORMOL, DIMETHYL OCTANOL PQ, DIMYRCETOL, DIOLA, DIPENTENE, DULCINYL® RECRYSTALLIZED, ETHYL-3-PHENYL GLYCIDATE, FLEURAMONE, FLEURANIL, FLORAL SUPER, FLORALOZONE, FLORIFFOL, FRAISTONE, FRUCTONE, GALAXOLIDE® 50, GALAXOLIDE® 50 BB, GALAXOLIDE® 50 IPM, GALAXOLIDE® UNDILUTED, GALBASCONE, GERALDEHYDE, GERANIOL 5020, GERANIOL 600 TYPE, GERANIOL 950, GERANIOL 980 (PURE), GERANIOL CFT COEUR, GERANIOL COEUR, GERANYL ACETATE COEUR, GERANYL ACETATE, PURE, GERANYL FORMATE, GRISALVA, GUAIYL ACETATE, HELIONAL™, HERBAL, HERBALIME™, HEXADECANOLIDE, HEXALON, HEXENYL SALICYLATE CIS 3-, HYACINTH BODY, HYACINTH BODY NO. 3, HYDRATROPIC ALDEHYDE.DMA, HYDROXYOL, INDOLAROME, INTRELEVEN ALDEHYDE, INTRELEVEN ALDEHYDE SPECIAL, IONONE ALPHA, IONONE BETA, ISO CYCLO CITRAL, ISO CYCLO GERANIOL, ISO E SUPER®, ISOBUTYL QUINOLINE, JASMAL, JESSEMAL®, KHARISMAL®, KHARISMAL® SUPER, KHUSINIL, KOAVONE®, KOHINOOL®, LIFFAROME™, LIMOXAL, LINDENOL™, LYRAL®, LYRAME SUPER, MANDARIN ALD 10% TRI ETH, CITR, MARITIMA, MCK CHINESE, MEIJIFF™, MELAFLEUR, MELOZONE, METHYL ANTHRANILATE, METHYL IONONE ALPHA EXTRA, METHYL IONONE GAMMA A, METHYL IONONE GAMMA COEUR, METHYL IONONE GAMMA PURE, METHYL LAVENDER KETONE, MONTAVERDI®, MUGUESIA, MUGUET ALDEHYDE 50, MUSK Z4, MYRAC ALDEHYDE, MYRCENYL ACETATE, NECTARATE™, NEROL 900, NERYL ACETATE, OCIMENE, OCTACETAL, ORANGE FLOWER ETHER, ORIVONE, ORRINIFF 25%, OXASPIRANE, OZOFLEUR, PAMPLEFLEUR®, PEOMOSA, PHENOXANOL®, PICONIA, PRECYCLEMONE B, PRENYL ACETATE, PRISMANTOL, RESEDA BODY, ROSALVA, ROSAMUSK, SANJINOL, SANTALIFF™, SYVERTAL, TERPINEOL,TERPINOLENE 20, TERPINOLENE 90 PQ, TERPINOLENE RECT., TERPINYL ACETATE, TERPINYL ACETATE JAX, TETRAHYDRO, MUGUOL®, TETRAHYDRO MYRCENOL, TETRAMERAN, TIMBERSILK™, TOBACAROL, TRIMOFIX® O TT, TRIPLAL®, TRISAMBER®, VANORIS, VERDOX™ VERDOX™ HC, VERTENEX®, VERTENEX® HC, VERTOFIX® COEUR, VERTOLIFF, VERTOLIFF ISO, VIOLIFF, VIVALDIE, ZENOLIDE, ABS INDIA 75 PCT MIGLYOL, ABS MOROCCO 50 PCT DPG, ABS MOROCCO 50 PCT TEC, ABSOLUTE FRENCH, ABSOLUTE INDIA, ABSOLUTE MD 50 PCT BB, ABSOLUTE MOROCCO, CONCENTRATE PG, TINCTURE 20 PCT, AMBERGRIS, AMBRETTE ABSOLUTE, AMBRETTE SEED OIL, ARMOISE OIL 70 PCT THUYONE, BASIL ABSOLUTE GRAND VERT, BASIL GRAND VERT ABS MD, BASIL OIL GRAND VERT, BASIL OIL VERVEINA, BASIL OIL VIETNAM, BAY OIL TERPENELESS, BEESWAX ABS N G, BEESWAX ABSOLUTE, BENZOIN RESINOID SIAM, BENZOIN RESINOID SIAM 50 PCT DPG, BENZOIN RESINOID SIAM 50 PCT PG, BENZOIN RESINOID SIAM 70.5 PCT TEC, BLACKCURRANT BUD ABS 65 PCT PG, BLACKCURRANT BUD ABS MD 37 PCT TEC, BLACKCURRANT BUD ABS MIGLYOL, BLACKCURRANT BUD ABSOLUTE BURGUNDY, BOIS DE ROSE OIL, BRAN ABSOLUTE, BRAN RESINOID, BROOM ABSOLUTE ITALY, CARDAMOM GUATEMALA CO2 EXTRACT, CARDAMOM OIL GUATEMALA, CARDAMOM OIL INDIA, CARROT HEART, CASSIE ABSOLUTE EGYPT, CASSIE ABSOLUTE MD 50 PCT IPM, CASTOREUM ABS 90 PCT TEC, CASTOREUM ABS C 50 PCT MIGLYOL, CASTOREUM ABSOLUTE, CASTOREUM RESINOID, CASTOREUM RESINOID 50 PCT DPG, CEDROL CEDRENE, CEDRUS ATLANTICA OIL REDIST, CHAMOMILE OIL ROMAN, CHAMOMILE OIL WILD, CHAMOMILE OIL WILD LOW LIMONENE, CINNAMON BARK OIL CEYLAN, CISTE ABSOLUTE, CISTE ABSOLUTE COLORLESS, CITRONELLA OIL ASIA IRON FREE, CIVET ABS 75 PCT PG, CIVET ABSOLUTE, CIVET TINCTURE 10 PCT, CLARY SAGE ABS FRENCH DECOL, CLARY SAGE ABSOLUTE FRENCH, CLARY SAGE C′LESS 50 PCT PG, CLARY SAGE OIL FRENCH, COPAIBA BALSAM, COPAIBA BALSAM OIL, CORIANDER SEED OIL, CYPRESS OIL, CYPRESS OIL ORGANIC, DAVANA OIL, GALBANOL, GALBANUM ABSOLUTE COLORLESS, GALBANUM OIL, GALBANUM RESINOID, GALBANUM RESINOID 50 PCT DPG, GALBANUM RESINOID HERCOLYN BHT, GALBANUM RESINOID TEC BHT, GENTIANE ABSOLUTE MD 20 PCT BB, GENTIANE CONCRETE, GERANIUM ABS EGYPT MD, GERANIUM ABSOLUTE EGYPT, GERANIUM OIL CHINA, GERANIUM OIL EGYPT, GINGER OIL 624, GINGER OIL RECTIFIED SOLUBLE, GUAIACWOOD HEART, HAY ABS MD 50 PCT BB, HAY ABSOLUTE, HAY ABSOLUTE MD 50 PCT TEC, HEALINGWOOD, HYSSOP OIL ORGANIC, IMMORTELLE ABS YUGO MD 50 PCT TEC, IMMORTELLE ABSOLUTE SPAIN, IMMORTELLE ABSOLUTE YUGO, JASMIN ABS INDIA MD, JASMIN ABSOLUTE EGYPT, JASMIN ABSOLUTE INDIA, ASMIN ABSOLUTE MOROCCO, JASMIN ABSOLUTE SAMBAC, JONQUILLE ABS MD 20 PCT BB, JONQUILLE ABSOLUTE France, JUNIPER BERRY OIL FLG, JUNIPER BERRY OIL RECTIFIED SOLUBLE, LABDANUM RESINOID 50 PCT TEC, LABDANUM RESINOID BB, LABDANUM RESINOID MD, LABDANUM RESINOID MD 50 PCT BB, LAVANDIN ABSOLUTE H, LAVANDIN ABSOLUTE MD, LAVANDIN OIL ABRIAL ORGANIC, LAVANDIN OIL GROSSO ORGANIC, LAVANDIN OIL SUPER, LAVENDER ABSOLUTE H, LAVENDER ABSOLUTE MD, LAVENDER OIL COUMARIN FREE, LAVENDER OIL COUMARIN FREE ORGANIC, LAVENDER OIL MAILLETTE ORGANIC, LAVENDER OIL MT, MACE ABSOLUTE BB, MAGNOLIA FLOWER OIL LOW METHYL EUGENOL, MAGNOLIA FLOWER OIL, MAGNOLIA FLOWER OIL MD, MAGNOLIA LEAF OIL, MANDARIN OIL MD, MANDARIN OIL MD BHT, MATE ABSOLUTE BB, MOSS TREE ABSOLUTE MD TEX IFRA 43, MOSS-OAK ABS MD TEC IFRA 43, MOSS-OAK ABSOLUTE IFRA 43, MOSS-TREE ABSOLUTE MD IPM IFRA 43, MYRRH RESINOID BB, MYRRH RESINOID MD, MYRRH RESINOID TEC, MYRTLE OIL IRON FREE, MYRTLE OIL TUNISIA RECTIFIED, NARCISSE ABS MD 20 PCT BB, NARCISSE ABSOLUTE FRENCH, NEROLI OIL TUNISIA, NUTMEG OIL TERPENELESS, OEILLET ABSOLUTE, OLIBANUM RESINOID, OLIBANUM RESINOID BB, OLIBANUM RESINOID DPG, OLIBANUM RESINOID EXTRA 50 PCT DPG, OLIBANUM RESINOID MD, OLIBANUM RESINOID MD 50 PCT DPG, OLIBANUM RESINOID TEC, OPOPONAX RESINOID TEC, ORANGE BIGARADE OIL MD BHT, ORANGE BIGARADE OIL MD SCFC, ORANGE FLOWER ABSOLUTE TUNISIA, ORANGE FLOWER WATER ABSOLUTE TUNISIA, ORANGE LEAF ABSOLUTE, ORANGE LEAF WATER ABSOLUTE TUNISIA, ORRIS ABSOLUTE ITALY, ORRIS CONCRETE 15 PCT IRONE, ORRIS CONCRETE 8 PCT IRONE, ORRIS NATURAL 15 PCT IRONE 4095C, ORRIS NATURAL 8 PCT IRONE 2942C, ORRIS RESINOID, OSMANTHUS ABSOLUTE, OSMANTHUS ABSOLUTE MD 50 PCT BB, PATCHOULI HEART N^{o}3, PATCHOULI OIL INDONESIA, PATCHOULI OIL INDONESIA IRON FREE, PATCHOULI OIL INDONESIA MD, PATCHOULI OIL REDIST, PENNYROYAL HEART, PEPPERMINT ABSOLUTE MD, PETITGRAIN BIGARADE OIL TUNISIA, PETITGRAIN CITRONNIER OIL, PETITGRAIN OIL PARAGUAY TERPENELESS, PETITGRAIN OIL TERPENELESS STAB, PIMENTO BERRY OIL, PIMENTO LEAF OIL, RHODINOL EX GERANIUM CHINA, ROSE ABS BULGARIAN LOW METHYL EUGENOL, ROSE ABS MOROCCO LOW METHYL EUGENOL, ROSE ABS TURKISH LOW METHYL EUGENOL, ROSE ABSOLUTE, ROSE ABSOLUTE BULGARIAN, ROSE ABSOLUTE DAMASCENA, ROSE ABSOLUTE MD, ROSE ABSOLUTE MOROCCO, ROSE ABSOLUTE TURKISH, ROSE OIL BULGARIAN, ROSE OIL DAMASCENA LOW METHYL EUGENOL, ROSE OIL TURKISH, ROSEMARY OIL CAMPHOR ORGANIC, ROSEMARY OIL TUNISIA, SANDALWOOD OIL INDIA, SANDALWOOD OIL INDIA RECTIFIED, SANTALOL, SCHINUS MOLLE OIL, ST JOHN BREAD TINCTURE 10 PCT, STYRAX RESINOID, STYRAX RESINOID, TAGETE OIL, TEA TREE HEART, TONKA BEAN ABS 50 PCT SOLVENTS, TONKA BEAN ABSOLUTE, TUBEROSE ABSOLUTE INDIA, VETIVER HEART EXTRA, VETIVER OIL HAITI, VETIVER OIL HAITI MD, VETIVER OIL JAVA, VETIVER OIL JAVA MD, VIOLET LEAF ABSOLUTE EGYPT, VIOLET LEAF ABSOLUTE EGYPT DECOL, VIOLET LEAF ABSOLUTE FRENCH, VIOLET LEAF ABSOLUTE MD 50 PCT BB, WORMWOOD OIL TERPENELESS, YLANG EXTRA OIL, YLANG III OIL and combinations of these.

The colorants can be among those listed in the Color Index International by the Society of Dyers and Colourists. Colorants include dyes and pigments and include those commonly used for coloring textiles, paints, inks and inkjet inks Some colorants that can be utilized include carotenoids, arylide yellows, diarylide yellows, β-naphthols, naphthols, benzimidazolones, disazo condensation pigments, pyrazolones, nickel azo yellow, phthalocyanines, quinacridones, perylenes and perinones, isoindolinone and isoindoline pigments, triarylcarbonium pigments, diketopyrrolo-pyrrole pigments, thioindigoids. Cartenoids include e.g., alpha-carotene, beta-carotene, gamma-carotene, lycopene, lutein and astaxanthin Annatto extract, Dehydrated beets (beet powder), Canthaxanthin, Caramel, Apo-8′-carotenal, Cochineal extract, Carmine, Sodium copper chlorophyllin, Toasted partially defatted cooked cottonseed flour, Ferrous gluconate, Ferrous lactate, Grape color extract, Grape skin extract (enocianina), Carrot oil, Paprika, Paprika oleoresin, Mica-based pearlescent pigments, Riboflavin, Saffron, Titanium dioxide, carbon black, self-dispersed carbon, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Potassium sodium copper chlorophyllin (chlorophyllin-copper complex), Dihydroxyacetone, Bismuth oxychloride, Ferric ammonium ferrocyanide, Ferric ferrocyanide, Chromium hydroxide green, Chromium oxide greens, Guanine, Pyrophyllite, Talc, Aluminum powder, Bronze powder, Copper powder, Zinc oxide, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3 (3), D&C Brown No. 1, Ext. D&C, Chromium-cobalt-aluminum oxide, Ferric ammonium citrate, Pyrogallol, Logwood extract, 1,4-Bis[(2-hydroxy-ethyl)amino]-9,10-anthracenedione bis(2-propenoic)ester copolymers, 1,4-Bis[(2-methylphenyl)amino]-9,10-anthracenedione, 1,4-Bis[4-(2-methacryloxyethyl)phenylamino]anthraquinone copolymers, Carbazole violet, Chlorophyllin-copper complex, Chromium-cobalt-aluminum oxide, C.I. Vat Orange 1, 2-[[2,5-Diethoxy-4-[(4-methylphenyl)thiol]phenyl]azo]-1,3,5-benzenetriol, 16,23-Dihydrodinaphtho[2,3-a:2′,3′-i]naphth[2′,3′:6,7]indolo[2,3-c]carbazole-5,10,15,17,22,24-hexone, N,N′-(9,10-Dihydro-9,10-dioxo-1,5-anthracenediyl)bisbenzamide, 7,16-Dichloro-6,15-dihydro-5,9,14,18-anthrazinetetrone, 16,17-Dimethoxydinaphtho (1,2,3-cd:3′,2′,1′-lm) perylene-5,10-dione, Poly(hydroxyethyl methacrylate)-dye copolymers(3), Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-2-phenyl-3H-pyrazol-3-one (solvent Yellow 18), 6-Ethoxy-2-(6-ethoxy-3-oxobenzo[b]thien-2(3H)-ylidene)benzo[b]thiophen-3(2H)-one, Phthalocyanine green, Vinyl alcohol/methyl methacrylate-dye reaction products, C.I. Reactive Red 180, C.I. Reactive Black 5, C.I. Reactive Orange 78, C.I. Reactive Yellow 15, C.I. Reactive Blue 21, Disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69), D&C Blue No. 9, [Phthalocyaninato(2-)]copper and mixtures of these.

For example, a fragrance, e.g., natural wood fragrance, can be compounded into the resin used to make the composite. In some implementations, the fragrance is compounded directly into the resin as an oil. For example, the oil can be compounded into the resin using a roll mill, e.g., a Banbury® mixer or an extruder, e.g., a twin-screw extruder with counter-rotating screws. An example of a Banbury® mixer is the F-Series Banbury® mixer, manufactured by Farrel. An example of a twin-screw extruder is the WP ZSK 50 MEGACOMPOUNDER™, manufactured by Coperion, Stuttgart, Germany. After compounding, the scented resin can be added to the fibrous material and extruded or molded. Alternatively, master batches of fragrance-filled resins are available commercially from International Flavors and Fragrances, under the trade name POLYIFF™. In some embodiments, the amount of fragrance in the composite is between about 0.005% by weight and about 10% by weight, e.g., between about 0.1% and about 5% or 0.25% and about 2.5%. Other natural wood fragrances include evergreen or redwood. Other fragrances include peppermint, cherry, strawberry, peach, lime, spearmint, cinnamon, anise, basil, bergamot, black pepper, camphor, chamomile, citronella, eucalyptus, pine, fir, geranium, ginger, grapefruit, jasmine, juniper berry, lavender, lemon, mandarin, marjoram, musk, myrrh, orange, patchouli, rose, rosemary, sage, sandalwood, tea tree, thyme, wintergreen, ylang ylang, vanilla, new car or mixtures of these fragrances. In some embodiments, the amount of fragrance in the fibrous material-fragrance combination is between about 0.005% by weight and about 20% by weight, e.g., between about 0.1% and about 5% or 0.25% and about 2.5%. Even other fragrances and methods are described U.S. Provisional Application Ser. No. 60/688,002, filed Jun. 7, 2005, the entire disclosure of which is hereby incorporated by reference herein.

### Uses of Polymers of Amino-Alpha, Omega-Dicarboxylic Acids and Copolymers

Some uses of polymers of amino-alpha, omega-dicarboxylic acid and polymers of amino-alpha, omega-dicarboxylic acid containing materials include: personal care items (e.g., tissues, towels, diapers), green packaging, garden (compostable pots), consumer electronics (e.g., laptop and mobile phone casings), appliances, food packaging, disposable packaging (e.g., food containers and drink bottles), garbage bags (e.g., waste compostable bags), mulch films, controlled release matrices and containers (e.g., for fertilizers, pesticides, herbicides, nutrients, pharmaceuticals, flavoring agents, foods), shopping bags, general purpose film, high heat film, heat seal layer, surface coating, disposable tableware (e.g., plates, cups, forks, knives, spoons, sporks, bowls), automotive parts (e.g., panels, fabrics, under hood covers), carpet fibers, clothing fibers (e.g., for garments, sportswear, footwear), biomedical applications (e.g., surgical sutures, implants, scaffolding, drug delivery systems, dialysis equipment) and engineering plastics.

Other uses/industrial sectors that can benefit from the use of polymers of amino-alpha, omega-dicarboxylic acid and polymers of amino-alpha, omega-dicarboxylic acid derivatives (e.g., elastomers) include IT and software, Electronics, geoscience (e.g., oil and gas), engineering, aerospace (e.g., arm rests, seats, panels), telecommunications (e.g., headsets), chemical manufacturing, transportation such as automotive (e.g., dashboards, panels, tires, wheels), materials and steel, consumer packaged goods, wires and cables.

### Other Advantages of Polymers of Amino-Alpha, Omega-Dicarboxylic Acids

Polymers of amino-alpha, omega-dicarboxylic acid can undergo hydrolytic degradation. Hydrolytic degradation includes chain scission producing shorter polymers, oligomers and eventually the monomer aspartic acid can be released. Hydrolysis can be associated with thermal and biotic degradation. The process can be effected by various parameters such as the polymers of amino-alpha, omega-dicarboxylic acid structure, its molecular weight and distribution, its morphology (e.g., crystallinity), the shape of the sample (e.g., isolated thin samples or comminuted samples can degrade faster), the thermal and mechanical history (e.g., processing) and the hydrolysis conditions (e.g., temperature, agitation, comminution). Polymers of amino-alpha, omega-dicarboxylic acid can also undergo biotic degradation. This degradation can occur for example, in a mammalian body, and has useful implications for degradable stitching and can have detrimental implications to other surgical implants. Enzymes, such as proteinase K and pronase can be utilized. Polymers of amino-alpha, omega-dicarboxylic acid can be bio-based and can be composted, recycled, used as a fuel (incinerated). Some of the degradation reactions include thermal degradation, hydrolytic degradation and biotic degradations.

During composting, polymers of amino-alpha, omega-dicarboxylic acid can go through several degradation stages. For example, an initial stage can occur due to exposure to moisture wherein the degradation is abiotic and the polymers of amino-alpha, omega-dicarboxylic acid degrades by hydrolysis. This stage can be accelerated by the presence of acids and bases and elevated temperatures. The first stage can lead to embrittlement of the polymer which can facilitate the diffusion of polymers of amino-alpha, omega-dicarboxylic acid out of the bulk polymers. The oligomers can then be attacked by micro-organisms. Organisms can degrade the oligomers and aspartic acid, leading to CO₂ and water. Time for this degradation can be on the order of about one to a few years depending on the factors previously mentioned. The degradation time is several orders of magnitude faster than typical petroleum based plastic such as polyethylene (e.g., on the order of hundreds of years).

Polymers of amino-alpha, omega-dicarboxylic acid can also be recycled. For example, the polymers of amino-alpha, omega-dicarboxylic acid can be hydrolyzed to the respective amino-alpha, omega-dicarboxylic acid, purified and re-polymerized Unlike other recyclable plastics such as PET and HDPE, polymers of amino-alpha, omega-dicarboxylic acid does not need to be down-graded to make a product of diminished value (e.g., from a bottle to decking or carpet). Polymers of amino-alpha, omega-dicarboxylic acid can be in theory recycled indefinitely. Optionally, polymers of amino-alpha, omega-dicarboxylic acid can be re-used and downgraded for several generations and then converted to polymers of amino-alpha, omega-dicarboxylic acid and re-polymerized.

Polymers of amino-alpha, omega-dicarboxylic acid can also be used as a fuel, for example, for energy production. Polymers of amino-alpha, omega-dicarboxylic acid can have high heat content e.g., up to about 8400 BTU. Incineration of pure polymers of amino-alpha, omega-dicarboxylic acid only releases carbon dioxide and water. Combinations with other ingredients typically amount to less than 1 ppm of non-polymers of amino-alpha, omega-dicarboxylic acid residuals (e.g., ash). Thus the combustion of polymers of amino-alpha, omega-dicarboxylic acid is cleaner than other renewable fuels, e.g. wood.

Processing as described herein can also include irradiation. For example, irradiation with between about 1 and 150 Mrad radiation (e.g., for example, any range as described herein) can improve the compostability and recyclability of polymers of amino-alpha, omega-dicarboxylic acid and polymers of amino-alpha, omega-dicarboxylic acid containing materials.

### Polymerization of Aspartic Acid and Polymeric Products

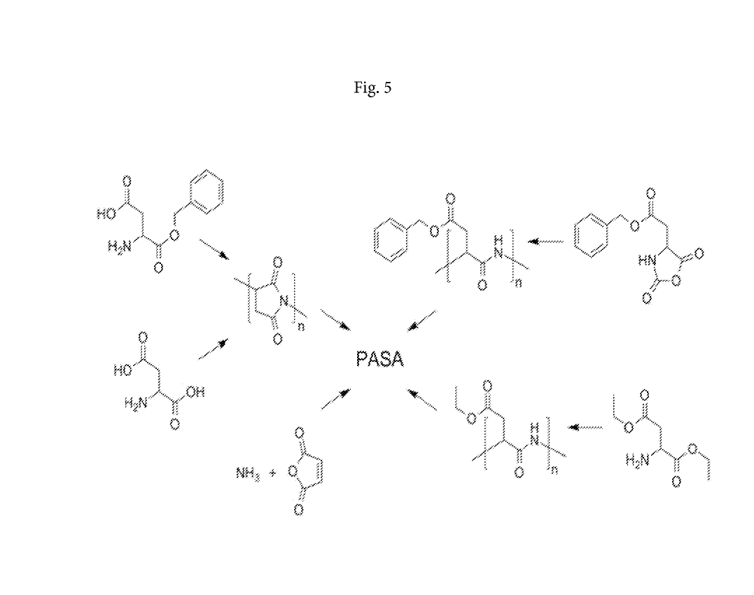

Polymers of aspartic acid are formed via many different polymerization schemes including the ones described above. Products include dimers, trimers, oligomers and polymers. One of these polymerization schemes results in a polyamide by amine condensation with one of the two carboxylic acids. A polyaspartic acid (PASA) is a polyamide with the amide linkage at the alpha and/or beta carboxylic acid. For PASA made via dehydration schemes the sodium-DL-(α,β)-poly(aspartate) with 30% α-linkages and 70% β-linkages randomly distributed along the polymer chain, and racemized chiral center of aspartic acid is produced. FIG. 5 shows candidate pathways to PASA.

There are many uses of PASA. For instance, it is used as a component in low volatile organic compounds coatings. In this case the low viscosity polyaspartic acids are cured with polysiocyanates to form a coating especially coatings for cars. A commercial example of this polyaspartic acid is Desmophen® NH 1420. Another example is an amphiphilic biodegradable copolymer based on a poly(aspartic acid-co-lactide). Polyaspartic acid may also be used as a non-toxic chelate composition in an aqueous fracturing fluid through chelation of ions. A pH sensitive hydrogel may be made from poly(aspartic acid) which is cross-linked with 1.6 hexanediamine and reinforced with ethyl cellulose. A lightly cross-linked polyaspartate can have high water absorbency and can be used as a superabsorbent. This use of lightly cross-linked polyaspartate is compared to poly (acrylic acid) but with improved biodegradability. Aspartic acid and/or polyaspartic acid may be used with polyalkylene glycol to produce a lubricant composition for automobile engines.

### Polymerization of Glutamic Acid and Polymeric Products

Polymers of glutamic acid are formed via many different polymerization schemes including the ones described above. Products include dimers, trimers, oligomers and polymers. One of these polymerization schemes results in a polyamide by amine condensation with one of the two carboxylic acids. A polyglutamic acid is a polyamide with the amide linkage at the alpha and/or gamma carboxylic acid. Bacillus subtilis, can be used to produce polyglutamic acid from devitalized wheat gluten. The gamma-polyglutamic acid is a water soluble and biodegradable polymer and biodegradable fibers and hydrogels. Gamma-polyglutamic acid also has use for skin care. It can be used as a replacement for hyaluronic acid.

There are many uses of polyglutamic acid. For instance, gamma-polyglutamic acid nanoparticles can be used for controlled anticancer drug release It has been reported that gamma-polyglutamic acid can be added to drinking water for chickens to improve calcium utilization.

### Radiation Treatment

The feedstock (e.g., cellulosic, lignocellulosic polymers of amino-alpha, omega-dicarboxylic acid, polymers of amino-alpha, omega-dicarboxylic acid derivatives and combinations of these) can be treated with electron bombardment to modify its structure, for example, to reduce its recalcitrance or cross link the structures. Such treatment can, for example, reduce the average molecular weight of the feedstock, change the crystalline structure of the feedstock, and/or increase the surface area and/or porosity of the feedstock. Alternatively, this treatment can produce radicals that can be sites for cross-linking, grafting and/or functionalization.

Electron bombardment via an electron beam is generally preferred, because it provides very high throughput. Accelerators used to accelerate the particles can be electrostatic DC, electrodynamic DC, RF linear, magnetic induction linear or continuous wave. For example, cyclotron type accelerators are available from IBA, Belgium, such as the RHODOTRON™ system, while DC type accelerators are available from RDI, now IBA Industrial, such as the DYNAMITRON®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 Mar. 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria.

Electron bombardment may be performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV.

The electron beam may have a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150, 250, 300 kW. In some cases, the power is even as high as 500 kW, 750 kW, or even 1000 kW or more. In some cases the electron beam has a beam power of 1200 kW or more, e.g., 1400, 1600, 1800, or even 3000 kW. The electron beam may have a total beam power of 25 to 3000 kW. Alternatively, the electron beam may have a total beam power of 75 to 1500 kW. Optionally, the electron beam may have a total beam power of 100 to 1000 kW. Alternatively, the electron beam may have a total beam power of 100 to 400 kW.

This high total beam power is usually achieved by utilizing multiple accelerating heads. For example, the electron beam device may include two, four, or more accelerating heads. The use of multiple heads, each of which has a relatively low beam power, prevents excessive temperature rise in the material, thereby preventing burning of the material, and also increases the uniformity of the dose through the thickness of the layer of material.

It is generally preferred that the bed of feedstock material has a relatively uniform thickness. In some embodiments the thickness is less than about 1 inch (e.g., less than about 0.75 inches, less than about 0.5 inches, less than about 0.25 inches, less than about 0.1 inches, between about 0.1 and 1 inch, between about 0.2 and 0.3 inches).

In some implementations, it is desirable to cool the material during and between dosing the material with electron bombardment. For example, the material can be cooled while it is conveyed, for example, by a screw extruder, vibratory conveyor or other conveying equipment. For example, cooling while conveying is described International App. No. PCT/US2014/021609 filed Mar. 7, 2014 and International App. No. PCT/US2014/021632 filed Mar. 7, 2014, the entire descriptions of which are herein incorporated by reference. To reduce the energy required by the recalcitrance-reducing process, it is desirable to treat the material as quickly as possible. In general, the treatment be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 30 Mrad per second. Alternately, the treatment is performed at a dose rate of 0.5 to 20 Mrad per second. Optionally, the treatment is performed at a dose rate of 0.75 to 15 Mrad per second. Alternately, the treatment is performed at a dose rate of 1 to 5 Mrad per second. Optionally, the treatment is performed at a dose rate of 1-3 Mrad per second or alternatively 1-2 Mrad per second. Higher dose rates allow a higher throughput for a target (e.g., the desired) dose. Higher dose rates generally require higher line speeds, to avoid thermal decomposition of the material. In one implementation, the accelerator is set for 3 MeV, 50 mA beam current, and the line speed is 24 feet/minute, for a sample thickness of about 20 mm (e g, comminuted corn cob material with a bulk density of 0.5 g/cm³).

In some embodiments, electron bombardment is performed until the material receives a total dose of at least 0.1 Mrad, 0.25 Mrad, 1 Mrad, 5 Mrad, e.g., at least 10, 20, 30 or at least 40 Mrad. In some embodiments, the treatment is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of seconds, e.g., at 5 Mrad/pass with each pass being applied for about one second. Applying a dose of greater than 7 to 8 Mrad/pass can in some cases cause thermal degradation of the feedstock material. Cooling can be applied before, after, or during irradiation. For example, the cooling methods, systems and equipment as described in the following applications can be utilized: International App. No. PCT/US2014/021609 filed Mar. 7, 2014, and International App. No. PCT/US2013/064320 filed Oct. 10, 2013, the entire disclosures of which are herein incorporated by reference.

Using multiple heads as discussed above, the material can be treated in multiple passes, for example, two passes at 10 to 20 Mrad/pass, e.g., 12 to 18 Mrad/pass, separated by a few seconds of cool-down, or three passes of 7 to 12 Mrad/pass, e.g., 5 to 20 Mrad/pass, 10 to 40 Mrad/pass, 9 to 11 Mrad/pass. As discussed herein, treating the material with several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance treatment uniformity.

In some embodiments, electrons are accelerated to, for example, a speed of greater than 75 percent of the speed of light, e.g., greater than 85, 90, 95, or 99 percent of the speed of light.

In some embodiments, any processing described herein occurs on feedstock material that remains dry as acquired or that has been dried, e.g., using heat and/or reduced pressure. For example, in some embodiments, the cellulosic and/or lignocellulosic material has less than about 25 wt. % retained water, measured at 25° C. and at fifty percent relative humidity (e.g., less than about 20 wt. %, less than about 15 wt. %, less than about 14 wt. %, less than about 13 wt. %, less than about 12 wt. %, less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, less than about 0.5 wt. %, less than about 15 wt. %.

In some embodiments, two or more electron sources are used, such as two or more ionizing sources. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light. The biomass is conveyed through the treatment zone where it can be bombarded with electrons.

It may be advantageous to repeat the treatment to more thoroughly reduce the recalcitrance of the biomass and/or further modify the biomass. In particular, the process parameters can be adjusted after a first (e.g., second, third, fourth or more) pass depending on the recalcitrance of the material. In some embodiments, a conveyor can be used which includes a circular system where the biomass is conveyed multiple times through the various processes described above. In some other embodiments, multiple treatment devices (e.g., electron beam generators) are used to treat the biomass multiple (e.g., 2, 3, 4 or more) times. In yet other embodiments, a single electron beam generator may be the source of multiple beams (e.g., 2, 3, 4 or more beams) that can be used for treatment of the biomass.

The effectiveness in changing the molecular/supermolecular structure and/or reducing the recalcitrance of the carbohydrate-containing biomass depends on the electron energy used and the dose applied, while exposure time depends on the power and dose. In some embodiments, the dose rate and total are adjusted so as not to destroy (e.g., char or burn) the biomass material. For example, the carbohydrates should not be damaged in the processing so that they can be released from the biomass intact, e.g. as monomeric sugars.

In some embodiments, the treatment (with any electron source or a combination of sources) is performed until the material receives a dose of at least about 0.05 Mrad, e.g., at least about 0.1, 0.25, 0.5, 0.75, 1.0, 2.5, 5.0, 7.5, 10.0, 15, 20, 25, 30, 40, 50, 60, 70, 80, 90, 100, 125, 150, 175, or 200 Mrad. In some embodiments, the treatment is performed until the material receives a dose of between 0.1-100 Mrad, 1-200, 5-200, 10-200, 5-150, 50-150 Mrad, 5-100, 5-50, 5-40, 10-50, 10-75, 15-50, 20-35 Mrad.

### Radiation Opaque Materials

The invention can include processing the material in a vault and/or bunker that is constructed using radiation opaque materials. In some implementations, the radiation opaque materials are selected to be capable of shielding the components from X-rays with high energy (short wavelength), which can penetrate many materials. One important factor in designing a radiation shielding enclosure is the attenuation length of the materials used, which will determine the required thickness for a particular material, blend of materials, or layered structure. The attenuation length is the penetration distance at which the radiation is reduced to approximately 1/e (e=Euler's number) times that of the incident radiation. Although virtually all materials are radiation opaque if thick enough, materials containing a high compositional percentage (e.g., density) of elements that have a high Z value (atomic number) have a shorter radiation attenuation length and thus, if such materials are used, a thinner, lighter shielding can be provided. Examples of high Z value materials that are used in radiation shielding are tantalum and lead. Another important parameter in radiation shielding is the halving distance, which is the thickness of a particular material that will reduce gamma ray intensity by 50%. As an example for X-ray radiation with an energy of 0.1 MeV the halving thickness is about 15.1 mm for concrete and about 0.27 mm for lead, while with an X-ray energy of 1 MeV the halving thickness for concrete is about 44.45 mm and for lead is about 7.9 mm Radiation opaque materials can be materials that are thick or thin so long as they can reduce the radiation that passes through to the other side. Thus, if it is desired that a particular enclosure have a low wall thickness, e.g., for light weight or due to size constraints, the material chosen should have a sufficient Z value and/or attenuation length so that its halving length is less than or equal to the desired wall thickness of the enclosure.

In some cases, the radiation opaque material may be a layered material, for example, having a layer of a higher Z value material, to provide good shielding, and a layer of a lower Z value material to provide other properties (e.g., structural integrity, impact resistance, etc.). In some cases, the layered material may be a “graded-Z” laminate, e.g., including a laminate in which the layers provide a gradient from high-Z through successively lower-Z elements. In some cases the radiation opaque materials can be interlocking blocks, for example, lead and/or concrete blocks can be supplied by NELCO Worldwide (Burlington, Mass.), and reconfigurable vaults can be utilized as described in International App. No. PCT/US2014/021629 filed on Mar. 7, 2014 the entire disclosure of which is herein incorporated by reference.

A radiation opaque material can reduce the radiation passing through a structure (e.g., a wall, door, ceiling, enclosure, a series of these or combinations of these) formed of the material by about at least about 10%, (e.g., at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%) as compared to the incident radiation. Therefore, an enclosure made of a radiation opaque material can reduce the exposure of equipment/system/components by the same amount. Radiation opaque materials can include stainless steel, metals with Z values above 25 (e.g., lead, iron), concrete, dirt, sand and combinations thereof. Radiation opaque materials can include a barrier in the direction of the incident radiation of at least about 1 mm (e.g., 5 mm, 10 mm, 5 cm, 10 cm, 100 cm, 1 m, 10 m).

### Electron Sources

Electrons interact via Coulomb scattering and bremsstrahlung radiation produced by changes in the velocity of electrons. Electrons may be produced by radioactive nuclei that undergo beta decay, such as isotopes of iodine, cesium, technetium, and iridium. Alternatively, an electron gun can be used as an electron source via thermionic emission and accelerated through an accelerating potential. An electron gun generates electrons, accelerates them through a large potential (e.g., greater than about 500 thousand, greater than about 1 million, greater than about 2 million, greater than about 5 million, greater than about 6 million, greater than about 7 million, greater than about 8 million, greater than about 9 million, or even greater than 10 million volts) and then scans them magnetically in the x-y plane, where the electrons are initially accelerated in the z direction down the tube and extracted through a foil window. Scanning the electron beam is useful for increasing the irradiation surface when irradiating materials, e.g., a biomass, that is conveyed through the scanned beam. Scanning the electron beam also distributes the thermal load homogenously on the window and helps reduce the foil window rupture due to local heating by the electron beam. Window foil rupture is a cause of significant down-time due to subsequent necessary repairs and re-starting the electron gun.

Various other irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, and folded tandem accelerators. Such devices are disclosed, for example, in U.S. Pat. No. 7,931,784 to Medoff, the complete disclosure of which is incorporated herein by reference.

A beam of electrons can be used as the radiation source. A beam of electrons has the advantages of high dose rates (e.g., 1, 5, or even 10 Mrad per second), high throughput, less containment, and less confinement equipment. Electron beams can also have high electrical efficiency (e.g., 80%), allowing for lower energy usage relative to other radiation methods, which can translate into a lower cost of operation and lower greenhouse gas emissions corresponding to the smaller amount of energy used. Electron beams can be generated, e.g., by electrostatic generators, cascade generators, transformer generators, low energy accelerators with a scanning system, low energy accelerators with a linear cathode, linear accelerators, and pulsed accelerators.

Electrons can also be more efficient at causing changes in the molecular structure of carbohydrate-containing materials, for example, by the mechanism of chain scission. In addition, electrons having energies of 0.5-10 MeV can penetrate low density materials, such as the biomass materials described herein, e.g., materials having a bulk density of less than 0.5 g/cm³, and a depth of 0.3-10 cm. Electrons as an ionizing radiation source can be useful, e.g., for relatively thin piles, layers or beds of materials, e.g., less than about 0.5 inch, e.g., less than about 0.4 inch, 0.3 inch, 0.25 inch, or less than about 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV. Methods of irradiating materials are discussed in U.S. Pat. App. Pub. 2012/0100577 A1, filed Oct. 18, 2011, the entire disclosure of which is herein incorporated by reference.

Electron beam irradiation devices may be procured commercially from Ion Beam Applications, Louvain-la-Neuve, Belgium or the Titan Corporation, San Diego, Calif. Typical electron energies can be 0.5 MeV, 1 MeV, 2 MeV, 4.5 MeV, 7.5 MeV, or 10 MeV. Typical electron beam irradiation device power can be 1 KW, 5 KW, 10 KW, 20 KW, 50 KW, 60 KW, 70 KW, 80 KW, 90 KW, 100 KW, 125 KW, 150 KW, 175 KW, 200 KW, 250 KW, 300 KW, 350 KW, 400 KW, 450 KW, 500 KW, 600 KW, 700 KW, 800 KW, 900 KW or even 1000 KW.

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Typically, generators are housed in a vault, e.g., of lead or concrete, especially for production from X-rays that are generated in the process. Tradeoffs in considering electron energies include energy costs.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available. The scanning beam is preferred in most embodiments describe herein because of the larger scan width and reduced possibility of local heating and failure of the windows.

### Electron Guns—Windows

The extraction system for an electron accelerator can include two window foils. Window foils are described in International App. No. PCT/US2013/064332 filed Oct. 10, 2013, the complete disclosure of which is herein incorporated by reference. The cooling gas in the two foil window extraction system can be a purge gas or a mixture, for example, air, or a pure gas. In one embodiment the gas is an inert gas such as nitrogen, argon, helium and or carbon dioxide. It is preferred to use a gas rather than a liquid since energy losses to the electron beam are minimized Mixtures of pure gas can also be used, either pre-mixed or mixed in line prior to impinging on the windows or in the space between the windows. The cooling gas can be cooled, for example, by using a heat exchange system (e.g., a chiller) and/or by using boil off from a condensed gas (e.g., liquid nitrogen, liquid helium).

When using an enclosure, the enclosed conveyor can also be purged with an inert gas so as to maintain an atmosphere at a reduced oxygen level. Keeping oxygen levels low avoids the formation of ozone which in some instances is undesirable due to its reactive and toxic nature. For example the oxygen can be less than about 20% (e.g., less than about 10%, less than about 1%, less than about 0.1%, less than about 0.01%, or even less than about 0.001% oxygen). Purging can be done with an inert gas including, but not limited to, nitrogen, argon, helium or carbon dioxide. This can be supplied, for example, from a boil off of a liquid source (e.g., liquid nitrogen or helium), generated or separated from air in situ, or supplied from tanks. The inert gas can be recirculated and any residual oxygen can be removed using a catalyst, such as a copper catalyst bed. Alternatively, combinations of purging, recirculating and oxygen removal can be done to keep the oxygen levels low.

The enclosure can also be purged with a reactive gas that can react with the biomass. This can be done before, during or after the irradiation process. The reactive gas can be, but is not limited to, nitrous oxide, ammonia, oxygen, ozone, hydrocarbons, aromatic compounds, amides, peroxides, azides, halides, oxyhalides, phosphides, phosphines, arsines, sulfides, thiols, boranes and/or hydrides. The reactive gas can be activated in the enclosure, e.g., by irradiation (e.g., electron beam, UV irradiation, microwave irradiation, heating, IR radiation), so that it reacts with the biomass. The biomass itself can be activated, for example by irradiation. Preferably the biomass is activated by the electron beam, to produce radicals which then react with the activated or unactivated reactive gas, e.g., by radical coupling or quenching.

Purging gases supplied to an enclosed conveyor can also be cooled, for example below about 25° C., below about 0° C., below about −40° C., below about −80° C., below about −120° C. For example, the gas can be boiled off from a compressed gas such as liquid nitrogen or sublimed from solid carbon dioxide. As an alternative example, the gas can be cooled by a chiller or part of or the entire conveyor can be cooled.

### Heating and Throughput During Radiation Treatment

Several processes can occur in biomass when electrons from an electron beam interact with matter in inelastic collisions. For example, ionization of the material, chain scission of polymers in the material, cross linking of polymers in the material, oxidation of the material, generation of X-rays (“Bremsstrahlung”) and vibrational excitation of molecules (e.g. phonon generation). Without being bound to a particular mechanism, the reduction in recalcitrance can be due to several of these inelastic collision effects, for example, ionization, chain scission of polymers, oxidation and phonon generation. Some of the effects (e.g., especially X-ray generation), necessitate shielding and engineering barriers, for example, enclosing the irradiation processes in a concrete (or other radiation opaque material) vault. Another effect of irradiation, vibrational excitation, is equivalent to heating up the sample. Heating the sample by irradiation can help in recalcitrance reduction, but excessive heating can destroy the material, as will be explained below.

The adiabatic temperature rise (ΔT) from adsorption of ionizing radiation is given by the equation: ΔT=D/Cp: where D is the average dose in KGy, Cp is the heat capacity in J/g ° C., and ΔT is the change in temperature in ° C. A typical dry biomass material will have a heat capacity close to 2. Wet biomass will have a higher heat capacity dependent on the amount of water since the heat capacity of water is very high (4.19 J/g ° C.). Metals have much lower heat capacities, for example, 304 stainless steel has a heat capacity of 0.5 J/g ° C. The temperature change due to the instant adsorption of radiation in a biomass and stainless steel for various doses of radiation is shown in Table 1.

TABLE 1
Calculated Temperature increase for biomass and stainless steel.

| Dose (Mrad) | Estimated Biomass ΔT (° C.) | Steel ΔT (° C.) |
|---|---|---|
| 10 | 50 | 200 |
| 50 | 250, Decomposition | 1000 |
| 100 | 500, Decomposition | 2000 |
| 150 | 750, Decomposition | 3000 |
| 200 | 1000, Decomposition | 4000 |

High temperatures can destroy and or modify the biopolymers in biomass so that the polymers (e.g., cellulose) are unsuitable for further processing. A biomass subjected to high temperatures can become dark, sticky and give off odors indicating decomposition. The stickiness can even make the material hard to convey. The odors can be unpleasant and be a safety issue. In fact, keeping the biomass below about 200° C. has been found to be beneficial in the processes described herein (e.g., below about 190° C., below about 180° C., below about 170° C., below about 160° C., below about 150° C., below about 140° C., below about 130° C., below about 120° C., below about 110° C., between about 60° C. and 180° C., between about 60° C. and 160° C., between about 60° C. and 150° C., between about 60° C. and 140° C., between about 60° C. and 130° C., between about 60° C. and 120° C., between about 80° C. and 180° C., between about 100° C. and 180° C., between about 120° C. and 180° C., between about 140° C. and 180° C., between about 160° C. and 180° C., between about 100° C. and 140° C., between about 80° C. and 120° C.).

It has been found that irradiation above about 10 Mrad is desirable for the processes described herein (e.g., reduction of recalcitrance). A high throughput is also desirable so that the irradiation does not become a bottle neck in processing the biomass. The treatment is governed by a Dose rate equation: M=FP/D*time, where M is the mass of irradiated material (Kg), F is the fraction of power that is adsorbed (unit less), P is the emitted power (KW=Voltage in MeV*Current in mA), time is the treatment time (sec) and D is the adsorbed dose (KGy). In an exemplary process where the fraction of adsorbed power is fixed, the Power emitted is constant and a set dosage is desired, the throughput (e.g., M, the biomass processed) can be increased by increasing the irradiation time. However, increasing the irradiation time without allowing the material to cool, can excessively heat the material as exemplified by the calculations shown above. Since biomass has a low thermal conductivity (less than about 0.1 Wm⁻¹K⁻¹), heat dissipation is slow, unlike, for example metals (greater than about 10 Wm⁻¹K⁻¹) which can dissipate energy quickly as long as there is a heat sink to transfer the energy to.

### Electron Guns—Beam Stops

In some embodiments the systems and methods include a beam stop (e.g., a shutter). For example, the beam stop can be used to quickly stop or reduce the irradiation of material without powering down the electron beam device. Alternatively the beam stop can be used while powering up the electron beam, e.g., the beam stop can stop the electron beam until a beam current of a desired level is achieved. The beam stop can be placed between the primary foil window and a secondary foil window. For example, the beam stop can be mounted so that it is movable, that is, so that it can be moved into and out of the beam path. Even partial coverage of the beam can be used, for example, to control the dose of irradiation. The beam stop can be mounted to the floor, to a conveyor for the biomass, to a wall, to the radiation device (e.g., at the scan horn), or to any structural support. Preferably the beam stop is fixed in relation to the scan horn so that the beam can be effectively controlled by the beam stop. The beam stop can incorporate a hinge, a rail, wheels, slots, or other means allowing for its operation in moving into and out of the beam. The beam stop can be made of any material that will stop at least 5% of the electrons, e.g., at least 10%, 20%, 30%, 40%, 50%, 60%, 70%, at least 80%, 85%, 90%, 91%, 92%, 93%, 94%, 95%, 96%, 97%, 98%, 99% or even about 100% of the electrons.

The beam stop can be made of a metal including, but not limited to, stainless steel, lead, iron, molybdenum, silver, gold, titanium, aluminum, tin, or alloys of these, or laminates (layered materials) made with such metals (e.g., metal-coated ceramic, metal-coated polymer, metal-coated composite, multilayered metal materials).

The beam stop can be cooled, for example, with a cooling fluid such as an aqueous solution or a gas. The beam stop can be partially or completely hollow, for example, with cavities. Interior spaces of the beam stop can be used for cooling fluids and gases. The beam stop can be of any shape, including flat, curved, round, oval, square, rectangular, beveled and wedged shapes.

The beam stop can have perforations so as to allow some electrons through, thus controlling (e.g., reducing) the levels of radiation across the whole area of the window, or in specific regions of the window. The beam stop can be a mesh formed, for example, from fibers or wires. Multiple beam stops can be used, together or independently, to control the irradiation. The beam stop can be remotely controlled, e.g., by radio signal or hard wired to a motor for moving the beam into or out of position.

### Biomass Materials

Lignocellulosic materials include, but are not limited to, wood (e.g., softwood, Pine softwood, Softwood, Softwood barks, Softwood stems, Spruce softwood, Hardwood, Willow Hardwood, aspen hardwood, Birch Hardwood, Hardwood barks, Hardwood stems, pine cones, pine needles), particle board, chemical pulps, mechanical pulps, paper, waste paper, forestry wastes (e.g., sawdust, aspen wood, wood chips, leaves), grasses including so-called energy grasses, (e.g., switchgrass, miscanthus, cord grass, reed canary grass, Coastal Bermuda grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair, nut shells, palm fronds and hulls and other palm byproducts), cotton, Cotton seed hairs, flax, sugar processing residues (e.g., bagasse, beet pulp, agave bagasse), algae, seaweed, manure (e.g., Solid cattle manure, Swine waste), sewage, carrot processing waste, molasses spent wash, alfalfa biver and mixtures of any of these.

In some cases, the lignocellulosic material includes corncobs. Ground or hammer milled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or cellulosic or lignocellulosic materials containing significant amounts of corncobs.

Corncobs, before and after comminution, are also easier to convey and disperse, and have a lesser tendency to form explosive mixtures in air than other cellulosic or lignocellulosic materials such as hay and grasses.

Cellulosic materials include, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high α-cellulose content such as cotton, and mixtures of any of these. For example, paper products as described in U.S. application Ser. No. 13/396,365 (“Magazine Feedstocks” by Medoff et al., filed Feb. 14, 2012), the full disclosure of which is incorporated herein by reference.

Cellulosic materials can also include lignocellulosic materials which have been partially or fully de-lignified.

In some instances other biomass materials can be utilized, for example, starchy materials. Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials. Mixtures of starchy, cellulosic and or lignocellulosic materials can also be used. For example, a biomass can be an entire plant, a part of a plant or different parts of a plant, e.g., a wheat plant, cotton plant, a corn plant, rice plant or a tree. The starchy materials can be treated by any of the methods described herein.

Microbial materials include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

In other embodiments, the biomass materials, such as cellulosic, starchy and lignocellulosic feedstock materials, can be obtained from transgenic microorganisms and plants that have been modified with respect to a wild type variety. Such modifications may be, for example, through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. application Ser. No. 13/396,369 filed Feb. 14, 2012 the full disclosure of which is incorporated herein by reference.

Any of the methods described herein can be practiced with mixtures of any biomass materials described herein.

### Biomass Material Preparation—Mechanical Treatments

The biomass can be in a dry form, for example, with less than about 35% moisture content (e.g., less than about 20%, less than about 15%, less than about 10% less than about 5%, less than about 4%, less than about 3%, less than about 2% or even less than about 1%). The biomass can also be delivered in a wet state, for example, as a wet solid, a slurry or a suspension with at least about 10 wt. % solids (e.g., at least about 20 wt. %, at least about 30 wt. %, at least about 40 wt. %, at least about 50 wt. %, at least about 60 wt. %, at least about 70 wt. %).

The processes disclosed herein can utilize low bulk density materials, for example, cellulosic or lignocellulosic feedstocks that have been physically pretreated to have a bulk density of less than about 0.75 g/cm³, e.g., less than about 0.7, 0.65, 0.60, 0.50, 0.35, 0.25, 0.20, 0.15, 0.10, 0.05 or less, e.g., less than about 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters. If desired, low bulk density materials can be densified, for example, by methods described in U.S. Pat. No. 7,971,809 to Medoff, the full disclosure of which is hereby incorporated by reference.

In some cases, the pre-treatment processing includes screening of the biomass material. Screening can be through a mesh or perforated plate with a desired opening size, for example, less than about 6.35 mm (¼ inch, 0.25 inch), (e.g., less than about 3.18 mm (⅛ inch, 0.125 inch), less than about 1.59 mm ( 1/16 inch, 0.0625 inch), is less than about 0.79 mm ( 1/32 inch, 0.03125 inch), e.g., less than about 0.51 mm ( 1/50 inch, 0.02000 inch), less than about 0.40 mm ( 1/64 inch, 0.015625 inch), less than about 0.23 mm (0.009 inch), less than about 0.20 mm ( 1/128 inch, 0.0078125 inch), less than about 0.18 mm (0.007 inch), less than about 0.13 mm (0.005 inch), or even less than about 0.10 mm ( 1/256 inch, 0.00390625 inch)). In one configuration the desired biomass falls through the perforations or screen and thus biomass larger than the perforations or screen are not irradiated. These larger materials can be re-processed, for example, by comminuting, or they can simply be removed from processing. In another configuration material that is larger than the perforations is irradiated and the smaller material is removed by the screening process or recycled. In this kind of a configuration, the conveyor itself (for example, a part of the conveyor) can be perforated or made with a mesh. For example, in one particular embodiment the biomass material may be wet and the perforations or mesh allow water to drain away from the biomass before irradiation.

Screening of material can also be by a manual method, for example by an operator or mechanoid (e.g., a robot equipped with a color, reflectivity or other sensor) that removes unwanted material. Screening can also be by magnetic screening wherein a magnet is disposed near the conveyed material and the magnetic material is removed magnetically.

Optional pre-treatment processing can include heating the material. For example, a portion of the conveyor can be sent through a heated zone. The heated zone can be created, for example, by IR radiation, microwaves, combustion (e.g., gas, coal, oil, biomass), resistive heating and/or inductive coils. The heat can be applied from at least one side or more than one side, can be continuous or periodic and can be for only a portion of the material or all the material. For example, a portion of the conveying trough can be heated by use of a heating jacket. Heating can be, for example, for the purpose of drying the material. In the case of drying the material, this can also be facilitated, with or without heating, by the movement of a gas (e.g., air, oxygen, nitrogen, He, CO₂, Argon) over and/or through the biomass as it is being conveyed.

Optionally, pre-treatment processing can include cooling the material. Cooling material is described in U.S. Pat. No. 7,900,857 to Medoff, the disclosure of which in incorporated herein by reference. For example, cooling can be by supplying a cooling fluid, for example, water (e.g., with glycerol), or nitrogen (e.g., liquid nitrogen) to the bottom of the conveying trough. Alternatively, a cooling gas, for example, chilled nitrogen can be blown over the biomass materials or under the conveying system.

Another optional pre-treatment processing method can include adding a material to the biomass. The additional material can be added by, for example, by showering, sprinkling and or pouring the material onto the biomass as it is conveyed. Materials that can be added include, for example, metals, ceramics and/or ions as described in U.S. Pat. App. Pub. 2010/0105119 A1 (filed Oct. 26, 2009) and U.S. Pat. App. Pub. 2010/0159569 A1 (filed Dec. 16, 2009), the entire disclosures of which are incorporated herein by reference. Optional materials that can be added include acids and bases. Other materials that can be added are oxidants (e.g., peroxides, chlorates), polymers, polymerizable monomers (e.g., containing unsaturated bonds), water, catalysts, enzymes and/or organisms. Materials can be added, for example, in pure form, as a solution in a solvent (e.g., water or an organic solvent) and/or as a solution. In some cases the solvent is volatile and can be made to evaporate e.g., by heating and/or blowing gas as previously described. The added material may form a uniform coating on the biomass or be a homogeneous mixture of different components (e.g., biomass and additional material). The added material can modulate the subsequent irradiation step by increasing the efficiency of the irradiation, damping the irradiation or changing the effect of the irradiation (e.g., from electron beams to X-rays or heat). The method may have no impact on the irradiation but may be useful for further downstream processing. The added material may help in conveying the material, for example, by lowering dust levels.

Biomass can be delivered to the conveyor (e.g., the vibratory conveyors used in the vaults herein described) by a belt conveyor, a pneumatic conveyor, a screw conveyor, a hopper, a pipe, manually or by a combination of these. The biomass can, for example, be dropped, poured and/or placed onto the conveyor by any of these methods. In some embodiments the material is delivered to the conveyor using an enclosed material distribution system to help maintain a low oxygen atmosphere and/or control dust and fines. Lofted or air suspended biomass fines and dust are undesirable because these can form an explosion hazard or damage the window foils of an electron gun (if such a device is used for treating the material).

The material can be leveled to form a uniform thickness between about 0.0312 and 5 inches (e.g., between about 0.0625 and 2.000 inches, between about 0.125 and 1 inches, between about 0.125 and 0.5 inches, between about 0.3 and 0.9 inches, between about 0.2 and 0.5 inches between about 0.25 and 1.0 inches, between about 0.25 and 0.5 inches, 0.100+/−0.025 inches, 0.150+/−0.025 inches, 0.200+/−0.025 inches, 0.250+/−0.025 inches, 0.300+/−0.025 inches, 0.350+/−0.025 inches, 0.400+/−0.025 inches, 0.450+/−0.025 inches, 0.500+/−0.025 inches, 0.550+/−0.025 inches, 0.600+/−0.025 inches, 0.700+/−0.025 inches, 0.750+/−0.025 inches, 0.800+/−0.025 inches, 0.850+/−0.025 inches, 0.900+/−0.025 inches, 0.900+/−0.025 inches.

Generally, it is preferred to convey the material as quickly as possible through the electron beam to maximize throughput. For example, the material can be conveyed at rates of at least 1 ft./min, e.g., at least 2 ft./min, at least 3 ft./min, at least 4 ft./min, at least 5 ft./min, at least 10 ft./min, at least 15 ft./min, 20, 25, 30, 35, 40, 45, 50 ft./min. The rate of conveying is related to the beam current, for example, for a ¼ inch thick biomass and 100 mA, the conveyor can move at about 20 ft./min to provide a useful irradiation dosage, at 50 mA the conveyor can move at about 10 ft./min to provide approximately the same irradiation dosage.

After the biomass material has been conveyed through the radiation zone, optional post-treatment processing can be done. The optional post-treatment processing can, for example, be a process described with respect to the pre-irradiation processing. For example, the biomass can be screened, heated, cooled, and/or combined with additives. Uniquely to post-irradiation, quenching of the radicals can occur, for example, quenching of radicals by the addition of fluids or gases (e.g., oxygen, nitrous oxide, ammonia, liquids), using pressure, heat, and/or the addition of radical scavengers. For example, the biomass can be conveyed out of the enclosed conveyor and exposed to a gas (e.g., oxygen) where it is quenched, forming carboxylated groups. In one embodiment the biomass is exposed during irradiation to the reactive gas or fluid. Quenching of biomass that has been irradiated is described in U.S. Pat. No. 8,083,906 to Medoff, the entire disclosure of which is incorporate herein by reference.

If desired, one or more mechanical treatments can be used in addition to irradiation to further reduce the recalcitrance of the carbohydrate-containing material. These processes can be applied before, during and or after irradiation.

In some cases, the mechanical treatment may include an initial preparation of the feedstock as received, e.g., size reduction of materials, such as by comminution, e.g., cutting, grinding, shearing, pulverizing or chopping. For example, in some cases, loose feedstock (e.g., recycled paper, starchy materials, or switchgrass) is prepared by shearing or shredding. Mechanical treatment may reduce the bulk density of the carbohydrate-containing material, increase the surface area of the carbohydrate-containing material and/or decrease one or more dimensions of the carbohydrate-containing material.

Alternatively, or in addition, the feedstock material can be treated with another treatment, for example, chemical treatments, such as with an acid (HCl, H₂SO₄, H₃PO₄), a base (e.g., KOH and NaOH), a chemical oxidant (e.g., peroxides, chlorates, ozone), irradiation, steam explosion, pyrolysis, sonication, oxidation, chemical treatment. The treatments can be in any order and in any sequence and combinations. For example, the feedstock material can first be physically treated by one or more treatment methods, e.g., chemical treatment including and in combination with acid hydrolysis (e.g., utilizing HCl, H₂SO₄, H₃PO₄), radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the structure of the material by mechanical treatment. As another example, a feedstock material can be conveyed through ionizing radiation using a conveyor as described herein and then mechanically treated. Chemical treatment can remove some or all of the lignin (for example, chemical pulping) and can partially or completely hydrolyze the material. The methods also can be used with pre-hydrolyzed material. The methods also can be used with material that has not been pre hydrolyzed The methods can be used with mixtures of hydrolyzed and non-hydrolyzed materials, for example, with about 50% or more non-hydrolyzed material, with about 60% or more non-hydrolyzed material, with about 70% or more non-hydrolyzed material, with about 80% or more non-hydrolyzed material or even with 90% or more non-hydrolyzed material.

In addition to size reduction, which can be performed initially and/or later in processing, mechanical treatment can also be advantageous for “opening up,” “stressing,” breaking or shattering the carbohydrate-containing materials, making the cellulose of the materials more susceptible to chain scission and/or disruption of crystalline structure during the physical treatment.

Methods of mechanically treating the carbohydrate-containing material include, for example, milling or grinding. Milling may be performed using, for example, a hammer mill, ball mill, colloid mill, conical or cone mill, disk mill, edge mill, Wiley mill, grist mill or other mill Grinding may be performed using, for example, a cutting/impact type grinder. Some exemplary grinders include stone grinders, pin grinders, coffee grinders, and burr grinders. Grinding or milling may be provided, for example, by a reciprocating pin or other element, as is the case in a pin mill Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

The milling of the biomass may be done either in a wet or dry state. The optimum condition can depend on the milling equipment, the biomass, whether subsequent steps are more suited to processing a dry material. The preferred liquid for the wet milling is water, and this can be done without additives like sulfur dioxide. Dry milling of the biomass may be a preferred process especially if subsequent treatments are better done is a dry state where the water content is less than about 15 weight percent, optionally less than 10 weight percent, or alternatively less than 5 weight percent. For example, the material can be wet and/or dry milled by the methods and equipment disclosed in U.S. Pat. No. 7,900,857, U.S. Pat. No. 8,420,356, and U.S. Pat. Application 2012/0315675 the full disclosures of which are incorporated herein by reference.

Mechanical feed preparation systems can be configured to produce streams with specific characteristics such as, for example, specific maximum sizes, specific length-to-width, or specific surface areas ratios. Physical preparation can increase the rate of reactions, improve the movement of material on a conveyor, improve the irradiation profile of the material, improve the radiation uniformity of the material, or reduce the processing time required by opening up the materials and making them more accessible to processes and/or reagents, such as reagents in a solution.

The bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state (e.g., after transport). The material can be densified, for example, from less than about 0.2 g/cc to more than about 0.9 g/cc (e.g., less than about 0.3 to more than about 0.5 g/cc, less than about 0.3 to more than about 0.9 g/cc, less than about 0.5 to more than about 0.9 g/cc, less than about 0.3 to more than about 0.8 g/cc, less than about 0.2 to more than about 0.5 g/cc). For example, the material can be densified by the methods and equipment disclosed in U.S. Pat. No. 7,932,065 to Medoff and International Publication No. WO 2008/073186 (which was filed Oct. 26, 2007, was published in English, and which designated the United States), the full disclosures of which are incorporated herein by reference. Densified materials can be processed by any of the methods described herein, or any material processed by any of the methods described herein can be subsequently densified.

In some embodiments, the material to be processed is in the form of a fibrous material that includes fibers provided by shearing a fiber source. For example, the shearing can be performed with a rotary knife cutter.

For example, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process.

For example, a rotary knife cutter can be used to concurrently shear the fiber source and screen the first fibrous material. A rotary knife cutter includes a hopper that can be loaded with a shredded fiber source prepared by shredding a fiber source.

In some implementations, the feedstock is physically treated prior to saccharification and/or fermentation. Physical treatment processes can include one or more of any of those described herein, such as mechanical treatment, chemical treatment, irradiation, sonication, oxidation, pyrolysis, heat treatment, or steam explosion. Treatment methods can be used in combinations of two, three, four, or even all of these technologies (in any order). When more than one treatment method is used, the methods can be applied at the same time or at different times. Other processes that change a molecular structure of a biomass feedstock may also be used, alone or in combination with the processes disclosed herein.

Mechanical treatments that may be used, and the characteristics of the mechanically treated carbohydrate-containing materials, are described in further detail in U.S. Pat. App. Pub. 2012/0100577 A1, filed Oct. 18, 2011, the full disclosure of which is hereby incorporated herein by reference.

The mechanical treatments described herein can also be applied to processing of PASA and PASA based materials.

### Sonication, Pyrolysis, Oxidation, Steam Explosion

If desired, one or more sonication, pyrolysis, oxidative, or steam explosion processes can be used instead of or in addition to irradiation to reduce or further reduce the recalcitrance of the carbohydrate-containing material or process PASA and/or PASA based materials. For example, these processes can be applied before, during and or after irradiation. These processes are described in detail in U.S. Pat. No. 7,932,065 to Medoff, the full disclosure of which is incorporated herein by reference.

### Heat Treatment of Biomass

Alternately, or in addition to the biomass may be heat treated for up to twelve hours at temperatures ranging from about 90° C. to about 160° C. Optionally, this heat treatment step is performed after biomass has been irradiated with an electron beam. The amount of time for the heat treatment is up to 9 hours, alternately up to 6 hours, optionally up to 4 hours and further up to about 2 hours. The treatment time can be up to as little as 30 minutes when the mass may be effectively heated.

The heat treatment can be performed 90° C. to about 160° C. or, optionally, at 100 to 150 or, alternatively, at 120 to 140° C. The biomass is suspended in water such that the biomass content is 10 to 75 wt. % in water. In the case of the biomass being the irradiated biomass water is added and the heat treatment performed.

The heat treatment is performed in an aqueous suspension or mixture of the biomass. The amount of biomass is 10 to 90 wt. % of the total mixture, alternatively 20 to 70 wt. % or optionally 25 to 50 wt. %. The irradiated biomass may have minimal water content so water must be added prior to the heat treatment.

Since at temperatures above 100° C. there will be pressure due at least in part to the vaporization of water, a pressure vessel can be utilized to accommodate and/or maintain the pressure. The process for the heat treatment may be batch, continuous, semi-continuous or other reactor configurations. The continuous reactor configuration may be a tubular reactor and may include device(s) within the tube which will facilitate heat transfer and mixing/suspension of the biomass. These tubular devices may include a one or more static mixers. The heat may also be put into the system by direct injection of steam.

### Conveying Systems

Various conveying systems can be used to convey the feedstock materials, for example, to a vault and under an electron beam in a vault. Exemplary conveyors are belt conveyors, pneumatic conveyors, screw conveyors, carts, trains, trains or carts on rails, elevators, front loaders, backhoes, cranes, various scrapers and shovels, trucks, and throwing devices can be used. For example, vibratory conveyors can be used in various processes described herein, for example, as disclosed in International App. No. PCT/US2013/064332 filed Oct. 10, 2013 the entire disclosure of which is herein incorporated by reference.

### Use of Treated Biomass Material

Using the methods described herein, a starting biomass material (e.g., plant biomass, animal biomass, paper, and municipal waste biomass) can be used as feedstock to produce useful intermediates and products such as organic acids, salts of organic acids, hydroxyl acids, PASA, acid anhydrides, esters of organic acids and fuels, e.g., fuels for internal combustion engines or feedstocks for fuel cells. Systems and processes are described herein that can use as feedstock cellulosic and/or lignocellulosic materials that are readily available, but often can be difficult to process, e.g., municipal waste streams and waste paper streams, such as streams that include newspaper, Kraft paper, corrugated paper or mixtures of these.

In order to convert the feedstock to a form that can be readily processed, the glucan- or xylan-containing cellulose in the feedstock can be hydrolyzed to low molecular weight carbohydrates, such as sugars, by a saccharifying agent, e.g., an enzyme or acid, a process referred to as saccharification. The low molecular weight carbohydrates can then be used, for example, in an existing manufacturing plant, such as a single cell protein plant, an enzyme manufacturing plant, or a fuel plant, e.g., an ethanol manufacturing facility.

The feedstock can be hydrolyzed using an enzyme, e.g., by combining the materials and the enzyme in a solvent, e.g., in an aqueous solution.

Alternatively, the enzymes can be supplied by organisms that break down biomass, such as the cellulose and/or the lignin portions of the biomass, contain or manufacture various cellulolytic enzymes (cellulases), ligninases or various small molecule biomass-degrading metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (beta-glucosidases).

During saccharification a cellulosic substrate can be initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose. The efficiency (e.g., time to hydrolyze and/or completeness of hydrolysis) of this process depends on the recalcitrance of the cellulosic material.

### Intermediates and Products

Using the processes described herein, the biomass material can be converted to one or more products, such as energy, fuels, foods and materials. Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols (e.g., containing greater than 10%, 20%, 30% or even greater than 40% water), biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives (e.g., fuel additives). Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha and beta unsaturated acids (e.g., acrylic acid) and olefins (e.g., ethylene). Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), and methyl or ethyl esters of any of these alcohols. Other products include methyl acrylate, methyl methacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, gamma-hydroxybutyric acid, and mixtures thereof, salts of any of these acids, mixtures of any of the acids and their respective salts.

Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be sanitized or sterilized prior to selling the products, e.g., after purification or isolation or even after packaging, to neutralize one or more potentially undesirable contaminants that could be present in the product(s). Such sanitation can be done with electron bombardment, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

Other intermediates and products, including food and pharmaceutical products, are described in U.S. Pat. App. Pub. 2010/0124583 A1, published May 20, 2010, to Medoff, the full disclosure of which is hereby incorporated by reference herein.

### Lignin Derived Products

The spent biomass (e.g., spent lignocellulosic material) from lignocellulosic processing by the methods described are expected to have a high lignin content and in addition to being useful for producing energy through combustion in a Co-Generation plant, may have uses as other valuable products. For example, the lignin can be used as captured as a plastic, or it can be synthetically upgraded to other plastics. In some instances, it can also be converted to lignosulfonates, which can be utilized as binders, dispersants, emulsifiers or as sequestrants.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

As a dispersant, the lignin or lignosulfonates can be used, e.g., concrete mixes, clay and ceramics, dyes and pigments, leather tanning and in gypsum board.

As an emulsifier, the lignin or lignosulfonates can be used, e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

For energy production lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be cross-linked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

Co-generation using spent biomass is described in International App. No. PCT/US2014/021634 filed Mar. 7, 2014, the entire disclosure therein is herein incorporated by reference.

Lignin derived products can also be combined with PASA and PASA derived products. (e.g., PASA that has been produced as described herein). For example, lignin and lignin derived products can be blended, grafted to or otherwise combined and/or mixed with PASA. The lignin can, for example, be useful for strengthening, plasticizing or otherwise modifying the PASA.

### Saccharification

The treated biomass materials can be saccharified, generally by combining the material and a cellulase enzyme in a fluid medium, e.g., an aqueous solution. In some cases, the material is boiled, steeped, or cooked in hot water prior to saccharification, as described in U.S. Pat. App. Pub. 2012/0100577 A1 by Medoff and Masterman, published on Apr. 26, 2012, the entire contents of which are incorporated herein.

The saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. The time required for complete saccharification will depend on the process conditions and the carbohydrate-containing material and enzyme used. If saccharification is performed in a manufacturing plant under controlled conditions, the cellulose may be substantially entirely converted to sugar, e.g., glucose in about 12-96 hours. If saccharification is performed partially or completely in transit, saccharification may take longer.

It is generally preferred that the tank contents be mixed during saccharification, e.g., using jet mixing as described in International App. No. PCT/US2010/035331, filed May 18, 2010, which was published in English as WO 2010/135380 and designated the United States, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, or amphoteric surfactants.

It is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. This reduces the volume to be shipped, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. Preferably the antimicrobial additive(s) are food-grade.

A relatively high concentration solution can be obtained by limiting the amount of water added to the carbohydrate-containing material with the enzyme. The concentration can be controlled, e.g., by controlling how much saccharification takes place. For example, concentration can be increased by adding more carbohydrate-containing material to the solution. In order to keep the sugar that is being produced in solution, a surfactant can be added, e.g., one of those discussed above. Solubility can also be increased by increasing the temperature of the solution. For example, the solution can be maintained at a temperature of 40-50° C., 60-80° C., or even higher.

### Saccharifying Agents

Suitable cellulolytic enzymes include cellulases from species in the genera Bacillus, Coprinus, Myceliophthora, Cephalosporium, Scytalidium, Penicillium, Aspergillus, Pseudomonas, Humicola, Fusarium, Thielavia, Acremonium, Chrysosporium and Trichoderma, especially those produced by a strain selected from the species Aspergillus (see, e.g., EP Pub. No. 0 458 162), Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp. (including, but not limited to, A. persicinum, A. acremonium, A. brachypenium, A. dichromosporum, A. obclavatum, A. pinkertoniae, A. roseogriseum, A. incoloratum, and A. furatum). Preferred strains include Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additional strains that can be used include, but are not limited to, Trichoderma (particularly T. viride, T. reesei, and T. koningii), alkalophilic Bacillus (see, for example, U.S. Pat. No. 3,844,890 and EP Pub. No. 0 458 162), and Streptomyces (see, e.g., EP Pub. No. 0 458 162).

In addition to or in combination to enzymes, acids, bases and other chemicals (e.g., oxidants) can be utilized to saccharify lignocellulosic and cellulosic materials. These can be used in any combination or sequence (e.g., before, after and/or during addition of an enzyme). For example strong mineral acids can be utilized (e.g. HCl, H₂SO₄, H₃PO₄) and strong bases (e.g., NaOH, KOH).

### Sugars

In the processes described herein, for example, after saccharification, sugars (e.g., glucose and xylose) can be isolated. For example, sugars can be isolated by precipitation, crystallization, chromatography (e.g., simulated moving bed chromatography, high pressure chromatography), centrifugation, extraction, any other isolation method known in the art, and combinations thereof.

### Fermentation

Yeast and Zymomonas bacteria, for example, can be used for fermentation or conversion of sugar(s) to alcohol(s). Other microorganisms are discussed below. The optimum pH for fermentations is about pH 4 to 7. For example, the optimum pH for yeast is from about pH 4 to 5, while the optimum pH for Zymomonas is from about pH 5 to 6. Typical fermentation times are about 24 to 168 hours (e.g., 24 to 96 hrs.) with temperatures in the range of 20° C. to 40° C. (e.g., 26° C. to 40° C.), however thermophilic microorganisms prefer higher temperatures.

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic condition, can be achieved or maintained by carbon dioxide production during the fermentation and no additional inert gas is needed.

In some embodiments, all or a portion of the fermentation process can be interrupted before the low molecular weight sugar is completely converted to a product (e.g., ethanol). The intermediate fermentation products include sugar and carbohydrates in high concentrations. The sugars and carbohydrates can be isolated via any means known in the art. These intermediate fermentation products can be used in preparation of food for human or animal consumption. Additionally or alternatively, the intermediate fermentation products can be ground to a fine particle size in a stainless-steel laboratory mill to produce a flour-like substance. Jet mixing may be used during fermentation, and in some cases saccharification and fermentation are performed in the same tank.

Nutrients for the microorganisms may be added during saccharification and/or fermentation, for example, the food-based nutrient packages described in U.S. Pat. App. Pub. 2012/0052536, filed Jul. 15, 2011, the complete disclosure of which is incorporated herein by reference.

“Fermentation” includes the methods and products that are disclosed in International App. No. PCT/US2012/071093 filed Dec. 20, 2012 and International App. No. PCT/US2012/071097 filed Dec. 12, 2012, the contents of both of which are incorporated by reference herein in their entirety.

Mobile fermenters can be utilized, as described in International App. No. PCT/US2007/074028 (which was filed Jul. 20, 2007, was published in English as WO 2008/011598 and designated the United States) and has a US issued U.S. Pat. No. 8,318,453, the contents of which are incorporated herein in its entirety. Similarly, the saccharification equipment can be mobile. Further, saccharification and/or fermentation may be performed in part or entirely during transit.

### Fermentation Agents

The microorganism(s) used in fermentation can be naturally-occurring microorganisms and/or engineered microorganisms. For example, the microorganism can be a bacterium (including, but not limited to, e.g., a cellulolytic bacterium), a fungus, (including, but not limited to, e.g., a yeast), a plant, a protist, e.g., a protozoa or a fungus-like protest (including, but not limited to, e.g., a slime mold), or an alga. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose bioconversion technology, in Handbook on Bioethanol: Production and Utilization, Wyman, C. E., ed., Taylor & Francis, Washington, D.C., 179-212)). Other suitable microorganisms include, for example, Zymomonas mobilis, Clostridium spp. (including, but not limited to, C. thermocellum (Philippidis, 1996, supra), C. saccharobutylacetonicum, C. tyrobutyricum C. saccharobutylicum, C. Puniceum, C. betjemckii, and C. acetobutylicum), Moniliella spp. (including but not limited to M. pollinis, M. tomentosa, M. madida, M nigrescens, M. oedocephali, M. megachiliensis), Yarrowia lipolytica, Aureobasidium sp., Trichosporonoides sp., Trigonopsis variabilis, Trichosporon sp., Moniliellaacetoabutans sp., Typhula variabilis, Candida magnoliae, Ustilaginomycetes sp., Pseudozyma tsukubaensis, yeast species of genera Zygosaccharomyces, Debaryomyces, Hansenula and Pichia, and fungi of the dematioid genus Torula (e.g., T. corallina).

Many such microbial strains are publicly available, either commercially or through depositories such as the ATCC (American Type Culture Collection, Manassas, Va., USA), the NRRL (Agricultural Research Service Culture Collection, Peoria, Ill., USA), or the DSMZ (Deutsche Sammlung von Mikroorganismen and Zellkulturen GmbH, Braunschweig, Germany), to name a few.

Commercially available yeasts include, for example, Red Star®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Hydrogenation and Other Chemical Transformations

The processes described herein can include hydrogenation. For example, glucose and xylose can be hydrogenated to sorbitol and xylitol respectively. Esters, for example, produced as described herein, can also be hydrogenated. Hydrogenation can be accomplished by use of a catalyst (e.g., Pt/gamma-Al₂O₃, Ru/C, Raney Nickel, copper chromite, or other catalysts know in the art) in combination with H₂ under high pressure (e.g., 10 to 12000 psi). Other types of chemical transformation of the products from the processes described herein can be used, for example, production of organic sugar derived products such (e.g., furfural and furfural-derived products). Chemical transformations of sugar derived products are described in U.S. Prov. App. No. 61/667,481, filed Jul. 3, 2012, the disclosure of which is incorporated herein by reference in its entirety.

### Hydrocarbon-Containing Materials

In other embodiments utilizing the methods and systems described herein, hydrocarbon-containing materials can be processed. Any process described herein can be used to treat any hydrocarbon-containing material herein described. “Hydrocarbon-containing materials,” as used herein, is meant to include oil sands, oil shale, tar sands, coal dust, coal slurry, bitumen, various types of coal, and other naturally-occurring and synthetic materials that include both hydrocarbon components and solid matter. The solid matter can include rock, sand, clay, stone, silt, drilling slurry, or other solid organic and/or inorganic matter. The term can also include waste products such as drilling waste and by-products, refining waste and by-products, or other waste products containing hydrocarbon components, such as asphalt shingling and covering, asphalt pavement, etc.

### OTHER EMBODIMENTS

Any material, processes or processed materials described herein can be used to make products and/or intermediates such as composites, fillers, binders, plastic additives, adsorbents and controlled release agents. The methods can include densification, for example, by applying pressure and heat to the materials. For example, composites can be made by combining fibrous materials with a resin or polymer (e.g., PASA). For example, radiation cross-linkable resin (e.g., a thermoplastic resin, PASA, and/or PASA derivatives) can be combined with a fibrous material to provide a fibrous material/cross-linkable resin combination. Such materials can be, for example, useful as building materials, protective sheets, containers and other structural materials (e.g., molded and/or extruded products). Absorbents can be, for example, in the form of pellets, chips, fibers and/or sheets. Adsorbents can be used, for example, as pet bedding, packaging material or in pollution control systems. Controlled release matrices can also be the form of, for example, pellets, chips, fibers and or sheets. The controlled release matrices can, for example, be used to release drugs, biocides, fragrances. For example, composites, absorbents and control release agents and their uses are described in U.S. Serial No. PCT/US2006/010648, filed Mar. 23, 2006, and U.S. Pat. No. 8,074,910 filed Nov. 22, 2011, the entire disclosures of which are herein incorporated by reference.

In some instances the biomass material is treated at a first level to reduce recalcitrance, e.g., utilizing accelerated electrons, to selectively release one or more sugars (e.g., xylose). The biomass can then be treated to a second level to release one or more other sugars (e.g., glucose). Optionally the biomass can be dried between treatments. The treatments can include applying chemical and biochemical treatments to release the sugars. For example, a biomass material can be treated to a level of less than about 20 Mrad (e.g., less than about 15 Mrad, less than about 10 Mrad, less than about 5 Mrad, less than about 2 Mrad) and then treated with a solution of sulfuric acid, containing less than 10% sulfuric acid (e.g., less than about 9%, less than about 8%, less than about 7%, less than about 6%, less than about 5%, less than about 4%, less than about 3%, less than about 2%, less than about 1%, less than about 0.75%, less than about 0.50%, less than about 0.25%) to release xylose. Xylose, for example, that is released into solution, can be separated from solids and optionally the solids washed with a solvent/solution (e.g., with water and/or acidified water). Optionally, the Solids can be dried, for example, in air and/or under vacuum optionally with heating (e.g., below about 150° C., below about 120° C.) to a water content below about 25 wt. % (below about 20 wt. %, below about 15 wt. %, below about 10 wt. %, below about 5 wt. %). The solids can then be treated with a level of less than about 30 Mrad (e.g., less than about 25 Mrad, less than about 20 Mrad, less than about 15 Mrad, less than about 10 Mrad, less than about 5 Mrad, less than about 1 Mrad or even not at all) and then treated with an enzyme (e.g., a cellulase) to release glucose. The glucose (e.g., glucose in solution) can be separated from the remaining solids. The solids can then be further processed, for example, utilized to make energy or other products (e.g., lignin derived products).

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

1. A method comprising:
treating a reduced recalcitrance lignocellulosic and/or cellulosic material with one or more enzymes and/or microorganisms to produce an amino-alpha, omega-dicarboxylic acid.

2. The method of claim 1 further comprising converting the amino-alpha, omega-dicarboxylic acid to product.

3. The method of claim 1 further comprising pretreating a feedstock with at least one of irradiation, sonication, oxidation, mechanical size reduction, pyrolysis and steam explosion to produce the reduced recalcitrance lignocellulosic and/or cellulosic material.

4. The method of claim 3 wherein irradiation is performed with an electron beam.

5. The method of claim 2 wherein converting the amino-alpha, omega-dicarboxylic acid to the product comprises chemically converting.

6. The method of claim 2 wherein converting the amino-alpha, omega-dicarboxylic acids to the product comprises biochemically converting.

7. The method of claim 5 wherein chemically converting is selected from the group consisting of polymerization, isomerization, esterification, amidation, cyclization, oxidation, reduction, disproportionation, phosgenation, and combinations thereof.

8. The method of claim 1 wherein treating is performed with one or more enzymes to release one or more sugars from the lignocellulosic and/or cellulosic material prior to producing the amino-alpha, omega-dicarboxylic acid.

9. The method of claim 1 wherein producing the amino-alpha, omega-dicarboxylic acid comprises treating initially to release one or more sugars from the lignocellulosic and/or cellulosic material followed by fermenting one of the sugars with the one or more of the microorganisms.

10. The method of claim 8 further comprising purifying the one or more sugars.

11. The method of claim 1 wherein the amino-alpha, omega-dicarboxylic acid is selected from the group consisting of aspartic acid, glutamic acid and the amino substituted malonic, adipic, pimelic, suberic, azelaic, sebacic, and substituted derivatives thereof.

12. The method of claim 11 wherein the amino-alpha, omega-dicarboxylic acid is aspartic acid or glutamic acid.

13. The method of claim 5 wherein converting comprises polymerizing the amino-alpha, omega-dicarboxylic acid to a polymer.

14. The method of claim 13 wherein a polymerizing method is selected from the group consisting of direct condensation of the amino-alpha, omega-dicarboxylic acid, azeotropic condensation of the amino-alpha, omega-dicarboxylic acid, and cyclization of the amino-alpha, omega-dicarboxylic acid followed by ring opening polymerization.

15. The method of claim 13 wherein the polymerizing further comprises coupling agents and/or chain extenders.

16. The method of claim 15 wherein the coupling agents and/or chain extenders are selected from the group consisting of phosgene, triphosgene, carbonyl diimidazole, dicyclohexylcarbodiimide, isocyanate, acid chlorides, acid anhydrides, epoxides, thiirane, oxazoline, orthoester, and combinations of these.

17. The method of claim 14 wherein the polymerization method is azeotropic condensation.

18. The method of claim 13 further comprising the utilization of catalysts and/or promoters selected from the group consisting of protonic acids, H₃PO₄, H₂SO₄, methane sulfonic acid, p-toluene sulfonic acid, supported sulfonic acid, metals, Mg, Al, Ti, Zn, Sn, metal oxides, TiO₂, ZnO, GeO₂, ZrO₂, SnO, SnO₂, Sb₂O₃, metal halides, ZnCl₂, SnCl₂, SnCl₄, Mn(AcO)₂, Fe₂(LA)₃, Co(AcO)₂, Ni(AcO)₂, Cu(OA)₂, Zn(LA)₂, Y(OA)₃, Al(i-PrO)₃, Ti(BuO)₄, TiO(acac)₂, (Bu)₂SnO and combinations of these.

19. The method of claim 13 further comprising conducting at least a portion of the polymerization at a temperature between about 100 and 240° C.

20. The method of claim 13 further comprising conducting at least a portion of the polymerization under vacuum.

21. The method of claim 14 wherein the polymerization method includes cyclizing the amino-alpha, omega-dicarboxylic acid followed by ring opening.

22. The method of claim 13 wherein converting further includes blending the polymer with a second polymer.

23. The method of claim 22 wherein the second polymer is selected from the group consisting of polyglycols, polyvinyl acetate, polyolefins, styrenic resins, polyacetals, poly(meth)acrylates, polycarbonate, polybutylene succinate, elastomers, polyurethanes, natural rubber, polybutadiene, neoprene, silicone, and combinations of these.

24. The method of claim 1 where the amino-alpha, omega-dicarboxylic acid amine group is reacted with a protecting group to form a protected amino-alpha, omega-dicarboxylic acid.

25. The method of claim 13 further comprising co-polymerizing the amino-alpha, omega-dicarboxylic acid with a monomer.

26. The method of claim 25 wherein the monomer is selected from the group consisting of elastomeric units, lactones, carbonates, morpholinediones, epoxides, 1,4-benzodioxepin-2,5-(3H)-dione Glycosalicylide, 1,4-benzodioxepin-2,5-(3H,3-methyl)-dione Lactosalicylide, dibenzo-1,5 dioxacin-6-12-dione disalicylide, morpholine-2,5-dione, 1,4-dioxane-2,5-dione glycolide, oxepane-2-one_{ε}-caprolactone, 1,3-dioxane-2-one trimethylene carconate, 2,2-dimethyltrimethylene carbonate, 1,5-dioxepane-2-one, 1,4-dioxane-2-one p-dioxanone, gamma-butyrolactone, beta-butyrolactone, beta-Me-delta-valerolactone, 1,4-dioxane-2,3-dione ethylene oxalate, 3-[benzyloxycarbonyl methyl]-1,4-dioxane-2,5-dione, ethylene oxide, propylene oxide, 5,5′(oxepane-2-one), 2,4,7,9-tetraoxaspiro[5,5]undecane-3,8-dione Spiro-bid-dimethylene caronate, diols and diamines and mixtures of these.

27. The method of claim 13 further comprising combining the polymer with fillers.

28. The method of claim 27 wherein the filler is selected from the group consisting of silicates, layered silicates, polymer and organically modified layered silicate, synthetic mica, carbon, carbon fibers, glass fibers, boric acid, talc, montmorillonite, clay, starch, corn starch, wheat starch, cellulose fibers, paper, rayon, non-woven fibers, wood flours, whiskers of potassium titanate, whiskers of aluminum borate, 4,4′-thiodiphenol, glycerol and combinations of these.

29. The method of claim 27 wherein combining further includes extrusion and/or compression molding.

30. The method of claim 13 further comprising cross linking the polymer.

31. The method of claim 30 wherein a cross linking agent is utilized to cross link the polymer and the cross-linking agent is selected from the group consisting of 5,5′-bis(oxepane-2-one)(bis-ε-caprolactone)), spiro-bis-dimethylene carbonate, peroxides, dicumyl peroxide, a,a′-bis(tert-butylperoxy)-diisopropylbenzene benzoyl peroxide, unsaturated alcohols, hydroxyethyl methacrylate, 2-butene-1,4-diol, unsaturated anhydrides, maleic anhydride, saturated epoxides, glycidyl methacrylate, irradiation and combinations of these.

32. The method of claim 13 further comprising processing the polymer by a method selected from injection molding, blow molding and thermoforming.

33. The method of claim 13 further comprising combining the polymer with a dye.

34. The method of claim 33 wherein the dye is selected from the group consisting of blue 3, blue 356, brown 1, orange 29, violet 26, violet 93, yellow 42, yellow 54, yellow 82 and combinations of these.

35. The method of claim 13 further comprising combining the polymer with a fragrance.

36. The method of claim 35 wherein the fragrance is selected from the group consisting of wood, evergreen, redwood, peppermint, cherry, strawberry, peach, lime, spearmint, cinnamon, anise, basil, bergamot, black pepper, camphor, chamomile, citronella, eucalyptus, pine, fir, geranium, ginger, grapefruit, jasmine, juniper berry, lavender, lemon, mandarin, marjoram, musk, myrrh, orange, patchouli, rose, rosemary, sage, sandalwood, tea tree, thyme, wintergreen, ylang ylang, vanilla, new car or mixtures of these fragrances.

37. The method of claim 35 wherein the fragrances are combined with the polymer in an amount between about 0.005% by weight and about 20% by weight.

38. The method of claim 13 wherein converting further includes blending the polymer with a plasticizer.

39. The method of claim 38 wherein the plasticizer is selected from the group consisting of triacetine, tributyl citrate, polyethylene glycol, fully acetylated monoglyceride based on fully hydrogenated castor oil, glycerine and acetic acid, diethyl bishydroxymethyl malonate and mixtures of these.

40. The method of any claim 13 further comprising grafting a molecule to the polymer.

41. The method of claim 40 wherein the molecule is selected from a monomer or a polymer.

42. The method of claim 40 further including at least one of the following: treating the polymer with a peroxide, heating above about 120° C., and irradiatio.

43. The method of claim 13 further comprising shaping, molding, carving, extruding and/or assembling the polymer into the product.

44. The method of claim 43 wherein the product is selected from the group consisting of personal care items, tissues, towels, diapers, green packaging, compostable pots, consumer electronics, laptop casings, mobile phone casings, appliances, food packaging, disposable packaging, food containers, drink bottles, garbage bags, waste compostable bags, mulch films, controlled release matrices, controlled release containers, containers for fertilizers, containers for pesticides, containers for herbicides, containers for nutrients, containers for pharmaceuticals, containers for flavoring agents, containers for foods, shopping bags, general purpose film, high heat film, heat seal layer, surface coating, disposable tableware, plates, cups, forks, knives, spoons, sporks, bowls, automotive parts, panels, fabrics, under hood covers, carpet fibers, clothing fibers, fibers for garments, fibers for sportswear, fibers for footwear, surgical sutures, implants, scaffolding and drug delivery systems.

45. The method of claim 43 wherein the product is selected from flavor enhancer, coatings, dispersants, superabsorbent, drug delivery systems, plant growth, metal chelator, waste water treatment, water treatment, and automotive additives.

46. A product comprising:
at least one converted amino-alpha, omega-dicarboxylic acid, wherein
the amino-alpha, omega-dicarboxylic acid is produced by the fermentation of sugars derived from the acidic or enzymatic saccharification of an irradiated lignocellulosic and/or cellulosic material.

47. The product of claim 46 wherein the amino-alpha, omega-dicarboxylic acid is selected from the group consisting of aspartic acid, glutamic acid and 2-aminoadipic acid.

48. The product of claim 46 wherein the product is a polymer including one or more of the converted amino-alpha, omega dicarboxylic acids in the polymer backbone.

49. The product of claim 48 further comprising a non-amino-alpha, omega-dicarboxylic acid in the polymer backbone.

50. The product of claim 48 wherein the polymer is cross-linked.

51. The product of claim 48 wherein the polymer is a graft co-polymer.

52. The product of claim 46 wherein the amino-alpha, omega-dicarboxylic acid is selected from the group consisting of aspartic acid, glutamic acid and mixtures thereof.

53. The product of claim 48 further comprising blending the polymer with a second polymer, a plasticizer, an elastomer, a fragrance, a dye, a pigment, a filler or a mixture of these.

54. A system for polymerization of an amino-alpha, omega-dicarboxylic acid comprising:
a reaction vessel, a screw extruder and a condenser;
a recirculating fluid flow path from an outlet of the reaction vessel to an inlet of the screw extruder and from an outlet of the screw extruder to an inlet of the reaction vessel, and
a fluid flow path from a second outlet of the reaction vessel to an inlet of the condenser.

55. The system of claim 54 further comprising a vacuum pump in fluid connection with the second fluid flow path for producing a vacuum in the second fluid flow path.

56. The system of claim 54 further comprising a control valve that in a first position provides a non-disrupted flow in the recirculating fluid flow path and in a second position provides a second fluid flow path.

57. The system of claim 56 wherein when the second fluid flow path is from the outlet of the reaction vessel to an inlet of a pelletizer.

58. The system of claim 56 wherein the second fluid flow path is from the outlet of the reaction vessel to the inlet of the extruder and from the outlet of the extruder to the inlet of a pelletizer.

59. A method of making a polymer or copolymer, the method comprising evaporating water as it is formed during condensation of an amino-alpha, omega-dicarboxylic acid polymer as it traverses a surface of a thin film evaporator.

60. The method of claim 59, where the thin film evaporator comprises a thin film polymerization/devolatilization device.

61. The method of claim 60, where an extruder is in fluid communication with the thin film polymerization/devolatilization device and the effluent of the extruder is the poly hydroxy-carboxylic acid polymer or the effluent of the extruder is recycled to the thin film evaporator.

62. The method of claim 61, where the extruder is a twin screw extruder.

63. The method of claim 59 wherein the amino-alpha, omega-dicarboxylic acid oligomer is derived from the monomer group consisting of D-aspartic acid, L-aspartic acid, D-glutamic acid, L-glutamic acid, and mixtures thereof.

64. The method of claim 59, where at least a part of the thin film evaporator operates at a temperature of 100 to 260° C.

65. The method of claim 59, where at least a part of the thin film evaporator operates at a pressure of 0.0001 torr or lower.

66. The method of claim 59, where prior to transferring the poly hydroxy-carboxylic acid to a thin film polymerization/devolatilization device or during operation of the thin film polymerization/devolatilization device a catalyst deactivator and/or stabilizer agent is added.

67. The method of claim 66, comprising removing the deactivated/stabilized catalyst prior to, during or after the thin film polymerization/devolatilization device by a filtration device.

68. The method of claim 67 where the filtration device is in fluid communication with the thin film polymerization/devolatilization device.

69. The method of claim 24 further comprising co-polymerizing the protected amino-alpha, omega-dicarboxylic acid with a monomer.
